(19) United States
(12) Patent Application Publication
(10) Pub. No.: US 2025/0163449 A1
(43) Pub. Date: May 22, 2025

(54) HERBICIDE-TOLERANT GENES AND METHOD FOR USING SAME

(71) Applicant: QINGDAO KINGAGROOT CHEMICAL COMPOUND CO., LTD., Qingdao, Shandong (CN)

(72) Inventors: Sudong MO, Qingdao, Shandong (CN); Junjie ZHANG, Qingdao, Shandong (CN); Yanmin LUO, Qingdao, Shandong (CN); Huarong LI, Qingdao, Shandong (CN)

(21) Appl. No.: 18/838,441

(22) PCT Filed: Feb. 6, 2023
(86) PCT No.: PCT/CN2023/074624

§ 371 Date: Aug. 14, 2024

(30) Foreign Application Priority Data
Feb. 25, 2022 (CN) 202210180516.3

(51) Int. Cl. C12N 15/82; A01N 43/40; A01P 13/02; C12N 9/02

(52) U.S. Cl. CPC C12N 15/8275; A01N 43/40; A01P 13/02; C12N 9/0071; C12N 15/8205; C12Y 114/11

(57) ABSTRACT

The present invention provides a polypeptide and recombinant DNA molecule suitable for conferring tolerance to pyridinyloxy acid herbicides, as well as a herbicide-tolerant plant, seed, cell and plant part containing the recombinant DNA molecule and the method of use thereof.

### CROSS-REFERENCE TO RELATED APPLICATIONS

This patent application is a U.S. National Stage of International Patent Application No. PCT/CN2023/074624, filed Feb. 6, 2024, which claims the benefit of Chinese Patent Application No. 202210180516.3, filed Feb. 25, 2022.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 435,883 Byte XML file named “771660_XML,” created on Aug. 13, 2024.

### TECHNICAL FIELD

The present invention generally relates to the field of biotechnology. More specifically, the present invention relates to a recombinant DNA molecule encoding an enzyme that degrades herbicides. The present invention also relates to a transgenic plant, part, seed, cell and plant part containing the recombinant DNA molecule and the method of use thereof.

### BACKGROUND OF THE INVENTION

Transgenic traits created by biotechnological methods are commonly used in agricultural crop production. A heterologous gene (also referred to as a transgene) is introduced into a plant to generate a transgenic trait. Expression of the transgene in the plant confers a desirable trait, such as herbicide tolerance, on the plant. Examples of transgenic herbicide-tolerant traits include glyphosate tolerance, glufosinate tolerance, and dicamba tolerance. With the increase of weed species that are resistant to the most commonly used herbicides, new herbicide-tolerant traits are needed in the field. Herbicides of particular interest are the pyridinyloxy acid herbicides. Pyridinyloxy acid herbicides provide control of a range of glyphosate-resistant weeds, thus resulting in a trait conferring herbicide tolerance that is particularly used in combination with other herbicide tolerance trait(s) in a cropping system.

The Sphingobium herbicidovorans strain MH isolated from a dichlorprop-degrading soil sample was identified as being capable of cleaving the ether bond of various phyenoxyalkanoic acid herbicides, thereby utilizing this as the sole source of carbon and energy for its growth (HPE Kohler, Journal of Industrial Microbiology & Biotechnology (1999) 23:336-340). Catabolism of the herbicides is carried out by two different enantioselective α-ketoglutarate-dependent dioxygenases, RdpA (R-dichlorprop dioxygenase) and SdpA (S-dichlorprop dioxygenase). (A Westendorf, et al., Microbiological Research (2002) 157:317-322; Westendorf, et al., ActaBiotechnologica (2003) 23 (1): 3-17). RdpA has been isolated from Sphingobium herbicidovorans (GenBank accessions AF516752 (DNA) and AAM90965 (protein)) and Delftia acidovorans (GenBank accessions NG-036924 (DNA) and YP-009083283 (protein)) (TA Mueller, et al., Applied and Environmental Microbiology (2004) 70 (10): 6066-6075). The RdpA and SdpA genes have been used for plant transformation to confer herbicide tolerance to crops (T R Wright, et al., Proceedings of the National Academy of Sciences USA, (2010) 107 (47): 20240-5). Improving the activity of the RdpA enzyme using protein engineering techniques to create a protein for use in transgenic plants would allow higher rates of herbicide application, thereby improving the safety of transgenic crops and weed control measures.

### BRIEF SUMMARY OF THE INVENTION

The present invention provides a recombinant DNA molecule comprising a nucleic acid sequence encoding a polypeptide, as compared with the amino acid sequence of RdpA as set forth in SEQ ID NO: 1, the amino acid sequence of the polypeptide has the following mutation: the amino acid at position 82 is mutated from leucine into histidine. In one embodiment, the amino acid sequence of the polypeptide also has one or more mutation(s) selected from the following groups: the amino acid at position 187 is mutated from valine into leucine, methionine or isoleucine; the amino acid at position 187 is mutated from valine into leucine, and the amino acid at position 104 is mutated from arginine into alanine, aspartic acid or leucine; the amino acid at position 187 is mutated from valine into leucine, and the amino acid at position 182 is mutated from phenylalanine into tryptophan; the amino acid at position 187 is mutated from valine into leucine, and the amino acid at position 103 is mutated from glycine into leucine; the amino acid at position 187 is mutated from valine into leucine, the amino acid at position 182 is mutated from phenylalanine into tryptophan, and the amino acid at position 104 is mutated from arginine into glycine; the amino acid at position 187 is mutated from valine into leucine, the amino acid at position 182 is mutated from phenylalanine into tryptophan, and the amino acid at position 103 is mutated from glycine into leucine; the amino acid at position 187 is mutated from valine into leucine, the amino acid at position 182 is mutated from phenylalanine into tryptophan, the amino acid at position 104 is mutated from arginine into glycine, and the amino acid at position 112 is mutated from threonine into serine; the amino acid at position 187 is mutated from valine into leucine, the amino acid at position 182 is mutated from phenylalanine into tryptophan, the amino acid at position 104 is mutated from arginine into glycine, and the amino acid at position 80 is mutated from valine into threonine; the amino acid at position 187 is mutated from valine into leucine, the amino acid at position 182 is mutated from phenylalanine into tryptophan, the amino acid at position 104 is mutated from arginine into glycine, and the amino acid at position 180 is mutated from arginine into tryptophan or methionine; the amino acid at position 187 is mutated from valine into leucine, the amino acid at position 182 is mutated from phenylalanine into tryptophan, the amino acid at position 104 is mutated from arginine into glycine, and the amino acid at position 108 is mutated from aspartic acid into cysteine; the amino acid at position 187 is mutated from valine into leucine, the amino acid at position 182 is mutated from phenylalanine into tryptophan, the amino acid at position 104 is mutated from arginine into glycine, and the amino acid at position 109 is mutated from aspartic acid into glutamic acid; the amino acid at position 187 is mutated from valine into leucine, the amino acid at position 182 is mutated from phenylalanine into tryptophan, the amino acid at position 104 is mutated from arginine into glycine, and the amino acid at position 219 is mutated from glutamine into cysteine or proline; the amino acid at position 187 is mutated from valine into leucine, the amino acid at position 182 is mutated from phenylalanine into tryptophan, the amino acid at position 103 is mutated from glycine into leucine, and the amino acid at position 180 is mutated from arginine into aspartic acid, glutamic acid, serine, leucine, tryptophan or threonine; the amino acid at position 187 is mutated from valine into leucine, the amino acid at position 182 is mutated from phenylalanine into tryptophan, the amino acid at position 103 is mutated from glycine into leucine, and the amino acid at position 80 is mutated from valine into threonine; the amino acid at position 187 is mutated from valine into leucine, the amino acid at position 182 is mutated from phenylalanine into tryptophan, the amino acid at position 103 is mutated from glycine into leucine, and the amino acid at position 112 is mutated from threonine into alanine, serine or methionine; the amino acid at position 187 is mutated from valine into leucine, the amino acid at position 182 is mutated from phenylalanine into tryptophan, the amino acid at position 103 is mutated from glycine into leucine, and the amino acid at position 247 is mutated from phenylalanine into tyrosine; the amino acid at position 187 is mutated from valine into leucine, the amino acid at position 182 is mutated from phenylalanine into tryptophan, the amino acid at position 104 is mutated from arginine into glycine, and the amino acid at position 77 is mutated from valine into isoleucine; the amino acid at position 187 is mutated from valine into leucine, the amino acid at position 182 is mutated from phenylalanine into tryptophan, the amino acid at position 104 is mutated from arginine into glycine, the amino acid at position 112 is mutated from threonine into serine, and the amino acid at position 180 is mutated from arginine into lysine, methionine, tryptophan or glutamine; and/or the amino acid at position 187 is mutated from valine into leucine, the amino acid at position 182 is mutated from phenylalanine into tryptophan, the amino acid at position 103 is mutated from glycine into leucine, the amino acid at position 104 is mutated from arginine into glycine, and the amino acid at position 105 is mutated from valine into tyrosine. In another embodiment, the amino acid sequence of the polypeptide further has at least 80%, at least 85%, at least 90%, at least 95%, at least 96%, at least 97%, at least 98%, at least 99% sequence identity to the amino acid sequence of RdpA as set forth in SEQ ID NO: 1.

The present invention also provides a recombinant DNA molecule comprising a nucleic acid sequence encoding a polypeptide that has at least about 92% sequence identity to an amino acid sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142. In another embodiment, the recombinant DNA molecule comprises a nucleic acid sequence selected from the group consisting of SEQ ID NO: 3, 4, 5, 7, 8, 9, 11, 12, 13, 15, 16, 17, 19, 20, 21, 23, 24, 25, 27, 28, 29, 31, 32, 33, 35, 36, 37, 39, 40, 41, 43, 44, 45, 47, 48, 49, 51, 52, 53, 55, 56, 57, 59, 60, 61, 63, 64, 65, 67, 68, 69, 71, 72, 73, 75, 76, 77, 79, 80, 81, 83, 84, 85, 87, 88, 89, 91, 92, 93, 95, 96, 97, 99, 100, 101, 103, 104, 105, 107, 108, 109, 111, 112, 113, 115, 116, 117, 119, 120, 121, 123, 124, 125, 127, 128, 129, 131, 132, 133, 135, 136, 137, 139, 140, 141 and 143-181, and a nucleic acid sequence encoding the same amino acid sequence as the sequence shown due to degeneracy of genetic code. In another embodiment, the recombinant DNA molecule encodes a polypeptide having oxygenase activity against at least one herbicide selected from the group consisting of pyridinyloxy acid herbicides. In another embodiment, the recombinant DNA molecule is operably linked to a heterologous promoter functional in a plant cell. In another embodiment, the recombinant DNA molecule is operably linked to a DNA molecule encoding a chloroplast transit peptide (CTP) that functions to localize an operably linked polypeptide within a cell.

The present invention provides a DNA construct comprising a heterologous promoter functional in a plant cell, and the heterologous promoter is operably linked to the recombinant DNA molecule of the present invention. In an embodiment, the recombinant DNA molecule is operably linked to a DNA molecule encoding a chloroplast transit peptide (CTP) that functions to localize an operably linked polypeptide within a cell. In another embodiment, the expression of the polypeptide in a transgenic plant encoded by the recombinant DNA molecule confers herbicide tolerance to the plant. In another embodiment, the DNA construct is present in the genome of a transgenic plant.

The present invention provides a transgenic plant, seed, cell or plant part comprising a recombinant DNA molecule of the present invention. In one embodiment, the transgenic plant, seed, cell or plant part comprises a transgenic trait for tolerance to at least one herbicide selected from the group consisting of pyridinyloxy acid herbicides. In another embodiment, the transgenic plant, seed, cell or plant part comprises a DNA construct of the present invention. In another embodiment, the transgenic plant, seed, cell or plant part comprises a polypeptide of the present invention.

The present invention provides a polypeptide, the amino acid sequence of which has the following mutation compared with the amino acid sequence of RdpA as set forth in SEQ ID NO: 1: the amino acid at position 82 is mutated from leucine into histidine. In one embodiment, the amino acid sequence of the polypeptide also has one or more mutation(s) as mentioned above. In another embodiment, the amino acid sequence of the polypeptide further has at least 80%, at least 85%, at least 90%, at least 95%, at least 96%, at least 97%, at least 98%, at least 99% sequence identity to the amino acid sequence of RdpA as set forth in SEQ ID NO: 1.

The present invention provides a polypeptide that has at least about 92% sequence identity to an amino acid sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142. In one embodiment, the polypeptide has oxygenase activity against at least one herbicide selected from the group consisting of pyridinyloxy acid herbicides.

The present invention provides a method for conferring herbicide tolerance to a plant, seed, cell or plant part comprising expressing a polypeptide of the present invention in the plant, seed, cell or plant part. In one embodiment, the method for conferring herbicide tolerance is used in combination with a transgenic plant, seed, cell or plant part that comprises a transgenic trait comprising a recombinant DNA molecule of the present invention. In one embodiment, the method for conferring herbicide tolerance is used in combination with a herbicide selected from the group consisting of pyridinyloxy acid herbicides.

The present invention provides a method of plant transformation comprising introducing a recombinant DNA molecule or DNA construct of the present invention into a plant cell or tissue, and regenerating a plant therefrom that comprises the recombinant DNA molecule or DNA construct and is tolerant to at least one herbicide selected from the group consisting of pyridinyloxy acid herbicides. In an embodiment, the method of plant transformation includes crossing the regenerated plant with itself or with a second plant and collecting seed from the cross.

The present invention provides a method for controlling weeds in a plant growing area by exposing a plant growing area comprising a transgenic plant or seed of the invention to at least one herbicide selected from the group consisting of pyridinyloxy acid herbicides, wherein the transgenic plant or seed is tolerant to the herbicide.

### BRIEF DESCRIPTION OF THE SEVERAL VIEWS OF THE DRAWING(S)

FIG. 1 shows control Jinjing 818 plants and transgenic Jinjing 818 plants comprising the protein-coding gene of SEQ ID NO: 42 after adding 0.5 μM and 1 μM compound B to medium for 19 days (variations in growth among plants that express the same protein may be resulted from changes in construct design or insertion position of the transgene, the same hereinafter).

FIG. 2 shows control Jinjing 818 plants and transgenic Jinjing 818 plants comprising the protein-coding gene of SEQ ID NO: 42 after applying 20 g, 40 g, 60 g, 80 g, 100 g, 120 g/mu compound B for corresponding days (DAT).

FIG. 3 shows wild-type control and T2 generation Arabidopsis thaliana plant seeds comprising the protein-coding gene of SEQ ID NO: 46 after adding 0.15 μM compound A to medium for 11 days of screening.

FIG. 4 shows wild-type control Arabidopsis thaliana plants transformed with RdpA gene and Arabidopsis thaliana plants comprising the protein-coding gene of SEQ ID NO: 42, after plants being foliage-sprayed with 40 g/mu compound B for 12 days.

FIG. 5 shows control Jinjing 818 plants and transgenic Jinjing 818 plants comprising the protein-coding genes of SEQ ID NO: 138/86 after adding 1 μM compound B to medium for 17 days.

FIG. 6 shows control Jinjing 818 plants and transgenic Jinjing 818 plants comprising the protein-coding gene of SEQ ID NO: 102 after adding 1 μM compound B to medium for 22 days.

FIG. 7 shows control Jinjing 818 plants and transgenic Jinjing 818 plants comprising the protein-coding gene of SEQ ID NO: 82 after adding 1 μM compound B to medium for 17 days.

FIG. 8 shows control Jinjing 818 plants and transgenic Jinjing 818 plants comprising the protein-coding gene of SEQ ID NO: 106 after adding 1 μM compound B to medium for 22 days.

FIG. 9 shows control Jinjing 818 plants and transgenic Jinjing 818 plants comprising the protein-coding gene of SEQ ID NO: 126 after adding 1 μM compound B to medium for 26 days.

FIG. 10 shows control Jinjing 818 plants and transgenic Jinjing 818 plants comprising the protein-coding gene of SEQ ID NO: 142 after adding 1 μM compound B to medium for 26 days.

FIG. 11 shows control Jinjing 818 plants and transgenic Jinjing 818 plants comprising the protein-coding gene of SEQ ID NO: 38 after adding 1 μM compound B to medium for 22 days.

FIG. 12 shows test results of control Jinjing 818 plants and transgenic Jinjing 818 plants comprising the protein-coding gene of SEQ ID NO: 46 after applying 0 g, 5 g, 10 g/mu compound quizalofop-P-ethyl for 20 DAT (framed ones in the picture are wild-type Jinjing 818 control plants, the rest are transgenic Jinjing 818 plants comprising the protein-coding gene of SEQ ID NO: 46).

FIG. 13 shows the root length comparison of T1 generation transgenic Jinjing 818 seeds comprising different protein-coding genes compared with wild-type Jinjing 818 after adding 0.3 μM compound B and soaking seeds for 20 days.

FIG. 14 shows the emergence comparison between T2 generation transgenic Arabidopsis thaliana seeds comprising the protein-coding gene of SEQ ID NO: 42 and wild-type Arabidopsis thaliana (the first line), after adding 0.15 μM compound A for 20-day screening.

FIG. 15 shows the emergence comparison between T2 generation transgenic Arabidopsis thaliana seeds comprising the protein-coding gene of SEQ ID NO: 78 and wild-type Arabidopsis thaliana (the first line), after adding 0.15 μM compound A for 19-day screening.

FIG. 16 shows the emergence comparison between T2 generation transgenic Arabidopsis thaliana seeds comprising the protein-coding gene of SEQ ID NO: 82 and wild-type Arabidopsis thaliana (the first line), after adding 0.15 μM compound A for 24-day screening.

FIG. 17 shows the emergence comparison between T2 generation transgenic Arabidopsis thaliana seeds comprising the protein-coding gene of SEQ ID NO: 86 and wild-type Arabidopsis thaliana (the first line), after adding 0.15 μM compound A for 21-day screening.

FIG. 18 shows the emergence comparison between T2 generation transgenic Arabidopsis thaliana seeds comprising the protein-coding gene of SEQ ID NO: 98 and wild-type Arabidopsis thaliana (the first line), after adding 0.15 μM compound A for 19-day screening.

FIG. 19 shows the emergence comparison between T2 generation transgenic Arabidopsis thaliana seeds comprising the protein-coding gene of SEQ ID NO: 102 and wild-type Arabidopsis thaliana (the first line), after adding 0.15 μM compound A for 19-day screening.

FIG. 20 shows the emergence comparison between T2 generation transgenic Arabidopsis thaliana seeds comprising the protein-coding gene of SEQ ID NO: 126 and wild-type Arabidopsis thaliana (the first line), after adding 0.15 μM compound A for 20-day screening.

FIG. 21 shows the emergence comparison between T2 generation transgenic Arabidopsis thaliana seeds comprising the protein-coding gene of SEQ ID NO: 138 and wild-type Arabidopsis thaliana (the first line), after adding 0.15 μM compound A for 19-day screening.

FIG. 22 shows the emergence comparison between T2 generation transgenic Arabidopsis thaliana seeds comprising the protein-coding gene of SEQ ID NO: 142 and wild-type Arabidopsis thaliana (the first line), after adding 0.15 μM compound A for 24-day screening.

FIG. 23 shows the resistance comparison between TO generation transgenic soybeans comprising the protein-coding gene of SEQ ID NO: 46 and a wild-type soybean (10 g compound C).

FIG. 24 shows the resistance comparison between T1 generation transgenic soybeans comprising the protein-coding gene of SEQ ID NO: 46 and a wild-type soybean (10 g compound C).

FIG. 25 shows the resistance comparison between T1 generation transgenic soybeans comprising the protein-coding gene of SEQ ID NO: 42 and wild-type soybeans (10 g compound C).

FIG. 26 shows the resistance comparison between T1 generation transgenic soybeans comprising the protein-coding gene of SEQ ID NO: 42 and wild-type soybeans (20 g, 40 g, 80 g compound C).

FIG. 27 shows test results of TO generation transgenic maize plantlets sprayed with 150 g, 250 g, 400 g, 600 g, 800 g of 30% glyphosate compound C (25+5) ME.

| | |
|---|---|
| SEQ ID NO: 1 | Amino acid sequence of RdpA |
| SEQ ID NO: 2 | Amino acid sequence of RdpA mutant L82H |
| SEQ ID NO: 3 | E. Coli codon optimized RdpA mutant L82H |
| SEQ ID NO: 4 | Maize codon optimized RdpA mutant L82H |
| SEQ ID NO: 5 | Cotton codon optimized RdpA mutant L82H |
| SEQ ID NO: 6 | Amino acid sequence of RdpA mutant L82H + V187L |
| SEQ ID NO: 7 | E. Coli codon optimized RdpA mutant L82H + V187L |
| SEQ ID NO: 8 | Maize codon optimized RdpA mutant L82H + V187L |
| SEQ ID NO: 9 | Cotton codon optimized RdpA mutant L82H + V187L |
| SEQ ID NO: 10 | Amino acid sequence of RdpA mutant L82H + V187M |
| SEQ ID NO: 11 | E. Coli codon optimized RdpA mutant L82H + V187M |
| SEQ ID NO: 12 | Maize codon optimized RdpA mutant L82H + V187M |
| SEQ ID NO: 13 | Cotton codon optimized RdpA mutant L82H + V187M |
| SEQ ID NO: 14 | Amino acid sequence of RdpA mutant L82H + V187I |
| SEQ ID NO: 15 | E. Coli codon optimized RdpA mutant L82H + V187I |
| SEQ ID NO: 16 | Maize codon optimized RdpA mutant L82H + V187I |
| SEQ ID NO: 17 | Cotton codon optimized RdpA mutant L82H + V187I |
| SEQ ID NO: 18 | Amino acid sequence of RdpA mutant L82H + V187L + R104A |
| SEQ ID NO: 19 | E. Coli codon optimized RdpA mutant L82H + V187L + R104A |
| SEQ ID NO: 20 | Maize codon optimized RdpA mutant L82H + V187L + R104A |
| SEQ ID NO: 21 | Cotton codon optimized RdpA mutant L82H + V187L + R104A |
| SEQ ID NO: 22 | Amino acid sequence of RdpA mutant L82H + V187L + R104D |
| SEQ ID NO: 23 | E. Coli codon optimized RdpA mutant L82H + V187L + R104D |
| SEQ ID NO: 24 | Maize codon optimized RdpA mutant L82H + V187L + R104D |
| SEQ ID NO: 25 | Cotton codon optimized RdpA mutant L82H + V187L + R104D |
| SEQ ID NO: 26 | Amino acid sequence of RdpA mutant L82H + V187L + R104L |
| SEQ ID NO: 27 | E. Coli codon optimized RdpA mutant L82H + V187L + R104L |
| SEQ ID NO: 28 | Maize codon optimized RdpA mutant L82H + V187L + R104L |
| SEQ ID NO: 29 | Cotton codon optimized RdpA mutant L82H + V187L + R104L |
| SEQ ID NO: 30 | Amino acid sequence of RdpA mutant L82H + V187L + F182W |
| SEQ ID NO: 31 | E. Coli codon optimized RdpA mutant L82H + V187L + F182W |
| SEQ ID NO: 32 | Maize codon optimized RdpA mutant L82H + V187L + F182W |
| SEQ ID NO: 33 | Cotton codon optimized RdpA mutant L82H + V187L + F182W |
| SEQ ID NO: 34 | Amino acid sequence of RdpA mutant L82H + V187L + G103L |
| SEQ ID NO: 35 | E. Coli codon optimized RdpA mutant L82H + V187L + G103L |
| SEQ ID NO: 36 | Maize codon optimized RdpA mutant L82H + V187L + G103L |
| SEQ ID NO: 37 | Cotton codon optimized RdpA mutant L82H + V187L + G103L |
| SEQ ID NO: 38 | Amino acid sequence of RdpA mutant L82H + V187L + F182W + R104G |
| SEQ ID NO: 39 | E. Coli codon optimized RdpA mutant L82H + V187L + F182W + R104G |
| SEQ ID NO: 40 | Maize codon optimized RdpA mutant L82H + V187L + F182W + R104G |
| SEQ ID NO: 41 | Cotton codon optimized RdpA mutant L82H + V187L + F182W + R104G |
| SEQ ID NO: 42 | Amino acid sequence of RdpA mutant L82H + V187L + F182W + G103L |
| SEQ ID NO: 43 | E. Coli codon optimized RdpA mutant L82H + V187L + F182W + G103L |
| SEQ ID NO: 44 | Maize codon optimized RdpA mutant L82H + V187L + F182W + G103L |
| SEQ ID NO: 45 | Cotton codon optimized RdpA mutant L82H + V187L + F182W + G103L |
| SEQ ID NO: 46 | Amino acid sequence of RdpA mutant L82H + V187L + F182W + R104G + T112S |
| SEQ ID NO: 47 | E. Coli codon optimized RdpA mutant L82H + V187L + F182W + R104G + T112S |
| SEQ ID NO: 48 | Maize codon optimized RdpA mutant L82H + V187L + F182W + R104G + T112S |
| SEQ ID NO: 49 | Cotton codon optimized RdpA mutant L82H + V187L + F182W + R104G + T112S |
| SEQ ID NO: 50 | Amino acid sequence of RdpA mutant L82H + V187L + F182W + R104G + V80T |
| SEQ ID NO: 51 | E. Coli codon optimized RdpA mutant L82H + V187L + F182W + R104G + V80T |
| SEQ ID NO: 52 | Maize codon optimized RdpA mutant L82H + V187L + F182W + R104G + V80T |
| SEQ ID NO: 53 | Cotton codon optimized RdpA mutant L82H + V187L + F182W + R104G + V80T |
| SEQ ID NO: 54 | Amino acid sequence of RdpA mutant L82H + V187L + F182W + R104G + R180W |
| SEQ ID NO: 55 | E. Coli codon optimized RdpA mutant L82H + V187L + F182W + R104G + R180W |
| SEQ ID NO: 56 | Maize codon optimized RdpA mutant L82H + V187L + F182W + R104G + R180W |
| SEQ ID NO: 57 | Cotton codon optimized RdpA mutant L82H + V187L + F182W + R104G + R180W |
| SEQ ID NO: 58 | Amino acid sequence of RdpA mutant L82H + V187L + F182W + R104G + R180M |
| SEQ ID NO: 59 | E. Coli codon optimized RdpA mutant L82H + V187L + F182W + R104G + R180M |
| SEQ ID NO: 60 | Maize codon optimized RdpA mutant L82H + V187L + F182W + R104G + R180M |
| SEQ ID NO: 61 | Cotton codon optimized RdpA mutant L82H + V187L + F182W + R104G + R180M |
| SEQ ID NO: 62 | Amino acid sequence of RdpA mutant L82H + V187L + F182W + R104G + D108C |
| SEQ ID NO: 63 | E. Coli codon optimized RdpA mutant L82H + V187L + F182W + R104G + D108C |
| SEQ ID NO: 64 | Maize codon optimized RdpA mutant L82H + V187L + F182W + R104G + D108C |
| SEQ ID NO: 65 | Cotton codon optimized RdpA mutant L82H + V187L + F182W + R104G + D108C |
| SEQ ID NO: 66 | Amino acid sequence of RdpA mutant L82H + V187L + F182W + R104G + D109E |
| SEQ ID NO: 67 | E. Coli codon optimized RdpA mutant L82H + V187L + F182W + R104G + D109E |
| SEQ ID NO: 68 | Maize codon optimized RdpA mutant L82H + V187L + F182W + R104G + D109E |
| SEQ ID NO: 69 | Cotton codon optimized RdpA mutant L82H + V187L + F182W + R104G + D109E |
| SEQ ID NO: 70 | Amino acid sequence of RdpA mutant L82H + V187L + F182W + R104G + Q219C |
| SEQ ID NO: 71 | E. Coli codon optimized RdpA mutant L82H + V187L + F182W + R104G + Q219C |
| SEQ ID NO: 72 | Maize codon optimized RdpA mutant L82H + V187L + F182W + R104G + Q219C |
| SEQ ID NO: 73 | Cotton codon optimized RdpA mutant L82H + V187L + F182W + R104G + Q219C |
| SEQ ID NO: 74 | Amino acid sequence of RdpA mutant L82H + V187L + F182W + R104G + Q219P |
| SEQ ID NO: 75 | E. Coli codon optimized RdpA mutant L82H + V187L + F182W + R104G + Q219P |
| SEQ ID NO: 76 | Maize codon optimized RdpA mutant L82H + V187L + F182W + R104G + Q219P |
| SEQ ID NO: 77 | Cotton codon optimized RdpA mutant L82H + V187L + F182W + R104G + Q219P |
| SEQ ID NO: 78 | Amino acid sequence of RdpA mutant L82H + V187L + F182W + G103L + R180D |
| SEQ ID NO: 79 | E. Coli codon optimized RdpA mutant L82H + V187L + F182W + G103L + R180D |
| SEQ ID NO: 80 | Maize codon optimized RdpA mutant L82H + V187L + F182W + G103L + R180D |
| SEQ ID NO: 81 | Cotton codon optimized RdpA mutant L82H + V187L + F182W + G103L + R180D |
| SEQ ID NO: 82 | Amino acid sequence of RdpA mutant L82H + V187L + F182W + G103L + R180E |
| SEQ ID NO: 83 | E. Coli codon optimized RdpA mutant L82H + V187L + F182W + G103L + R180E |
| SEQ ID NO: 84 | Maize codon optimized RdpA mutant L82H + V187L + F182W + G103L + R180E |
| SEQ ID NO: 85 | Cotton codon optimized RdpA mutant L82H + V187L + F182W + G103L + R180E |
| SEQ ID NO: 86 | Amino acid sequence of RdpA mutant L82H + V187L + F182W + G103L + R180S |
| SEQ ID NO: 87 | E. Coli codon optimized RdpA mutant L82H + V187L + F182W + G103L + R180S |
| SEQ ID NO: 88 | Maize codon optimized RdpA mutant L82H + V187L + F182W + G103L + R180S |
| SEQ ID NO: 89 | Cotton codon optimized RdpA mutant L82H + V187L + F182W + G103L + R180S |
| SEQ ID NO: 90 | Amino acid sequence of RdpA mutant L82H + V187L + F182W + G103L + R180L |
| SEQ ID NO: 91 | E. Coli codon optimized RdpA mutant L82H + V187L + F182W + G103L + R180L |
| SEQ ID NO: 92 | Maize codon optimized RdpA mutant L82H + V187L + F182W + G103L + R180L |
| SEQ ID NO: 93 | Cotton codon optimized RdpA mutant L82H + V187L + F182W + G103L + R180L |
| SEQ ID NO: 94 | Amino acid sequence of RdpA mutant L82H + V187L + F182W + G103L + R180W |
| SEQ ID NO: 95 | E. Coli codon optimized RdpA mutant L82H + V187L + F182W + G103L + R180W |
| SEQ ID NO: 96 | Maize codon optimized RdpA mutant L82H + V187L + F182W + G103L + R180W |
| SEQ ID NO: 97 | Cotton codon optimized RdpA mutant L82H + V187L + F182W + G103L + R180W |
| SEQ ID NO: 98 | Amino acid sequence of RdpA mutant L82H + V187L + F182W + G103L + V80T |
| SEQ ID NO: 99 | E. Coli codon optimized RdpA mutant L82H + V187L + F182W + G103L + V80T |
| SEQ ID NO: 100 | Maize codon optimized RdpA mutant L82H + V187L + F182W + G103L + V80T |
| SEQ ID NO: 101 | Cotton codon optimized RdpA mutant L82H + V187L + F182W + G103L + V80T |
| SEQ ID NO: 102 | Amino acid sequence of RdpA mutant L82H + V187L + F182W + G103L + T112A |
| SEQ ID NO: 103 | E. Coli codon optimized RdpA mutant L82H + V187L + F182W + G103L + T112A |
| SEQ ID NO: 104 | Maize codon optimized RdpA mutant L82H + V187L + F182W + G103L + T112A |
| SEQ ID NO: 105 | Cotton codon optimized RdpA mutant L82H + V187L + F182W + G103L + T112A |
| SEQ ID NO: 106 | Amino acid sequence of RdpA mutant L82H + V187L + F182W + G103L + T112S |
| SEQ ID NO: 107 | E. Coli codon optimized RdpA mutant L82H + V187L + F182W + G103L + T112S |
| SEQ ID NO: 108 | Maize codon optimized RdpA mutant L82H + V187L + F182W + G103L + T112S |
| SEQ ID NO: 109 | Cotton codon optimized RdpA mutant L82H + V187L + F182W + G103L + T112S |
| SEQ ID NO: 110 | Amino acid sequence of RdpA mutant L82H + V187L + F182W + G103L + T112M |
| SEQ ID NO: 111 | E. Coli codon optimized RdpA mutant L82H + V187L + F182W + G103L + T112M |
| SEQ ID NO: 112 | Maize codon optimized RdpA mutant L82H + V187L + F182W + G103L + T112M |
| SEQ ID NO: 113 | Cotton codon optimized RdpA mutant L82H + V187L + F182W + G103L + T112M |
| SEQ ID NO: 114 | Amino acid sequence of RdpA mutant L82H + V187L + F182W + G103L + F247Y |
| SEQ ID NO: 115 | E. Coli codon optimized RdpA mutant L82H + V187L + F182W + G103L + F247Y |
| SEQ ID NO: 116 | Maize codon optimized RdpA mutant L82H + V187L + F182W + G103L + F247Y |
| SEQ ID NO: 117 | Cotton codon optimized RdpA mutant L82H + V187L + F182W + G103L + F247Y |
| SEQ ID NO: 118 | Amino acid sequence of RdpA mutant L82H + V187L + F182W + R104G + V77I |
| SEQ ID NO: 119 | E. Coli codon optimized RdpA mutant L82H + V187L + F182W + R104G + V77I |
| SEQ ID NO: 120 | Maize codon optimized RdpA mutant L82H + V187L + F182W + R104G + V77I |
| SEQ ID NO: 121 | Cotton codon optimized RdpA mutant L82H + V187L + F182W + R104G + V77I |
| SEQ ID NO: 122 | Amino acid sequence of RdpA mutant L82H + V187L + F182W + G103L + R104G + V105Y |
| SEQ ID NO: 123 | E. Coli codon optimized RdpA mutant L82H + V187L + F182W + G103L + R104G + V105Y |
| SEQ ID NO: 124 | Maize codon optimized RdpA mutant L82H + V187L + F182W + G103L + R104G + V105Y |
| SEQ ID NO: 125 | Cotton codon optimized RdpA mutant L82H + V187L + F182W + G103L + R104G + V105Y |
| SEQ ID NO: 126 | Amino acid sequence of RdpA mutant L82H + V187L + F182W + R104G + T112S + R180K |
| SEQ ID NO: 127 | E. Coli codon optimized RdpA mutant L82H + V187L + F182W + R104G + T112S + R180K |
| SEQ ID NO: 128 | Maize codon optimized RdpA mutant L82H + V187L + F182W + R104G + T112S + R180K |
| SEQ ID NO: 129 | Cotton codon optimized RdpA mutant L82H + V187L + F182W + R104G + T112S + R180K |
| SEQ ID NO: 130 | Amino acid sequence of RdpA mutant L82H + V187L + F182W + R104G + T112S + R180M |
| SEQ ID NO: 131 | E. Coli codon optimized RdpA mutant L82H + V187L + F182W + R104G + T112S + R180M |
| SEQ ID NO: 132 | Maize codon optimized RdpA mutant L82H + V187L + F182W + R104G + T112S + R180M |
| SEQ ID NO: 133 | Cotton codon optimized RdpA mutant L82H + V187L + F182W + R104G + T112S + R180M |
| SEQ ID NO: 134 | Amino acid sequence of RdpA mutant L82H + V187L + F182W + R104G + T112S + R180W |
| SEQ ID NO: 135 | E. Coli codon optimized RdpA mutant L82H + V187L + F182W + R104G + T112S + R180W |
| SEQ ID NO: 136 | Maize codon optimized RdpA mutant L82H + V187L + F182W + R104G + T112S + R180W |
| SEQ ID NO: 137 | Cotton codon optimized RdpA mutant L82H + V187L + F182W + R104G + T112S + R180W |
| SEQ ID NO: 138 | Amino acid sequence of RdpA mutant L82H + V187L + F182W + G103L + R180T |
| SEQ ID NO: 139 | E. Coli codon optimized RdpA mutant L82H + V187L + F182W + G103L + R180T |
| SEQ ID NO: 140 | Maize codon optimized RdpA mutant L82H + V187L + F182W + G103L + R180T |
| SEQ ID NO: 141 | Cotton codon optimized RdpA mutant L82H + V187L + F182W + G103L + R180T |
| SEQ ID NO: 142 | Amino acid sequence of RdpA mutant L82H + V187L + F182W + R104G + T112S + R180Q |
| SEQ ID NO: 143 | E. Coli codon optimized RdpA mutant L82H + V187L + F182W + R104G + T112S + R180Q |
| SEQ ID NO: 144 | Maize codon optimized RdpA mutant L82H + V187L + F182W + R104G + T112S + R180Q |
| SEQ ID NO: 145 | Cotton codon optimized RdpA mutant L82H + V187L + F182W + R104G + T112S + R180Q |
| SEQ ID NO: 146 | Soybean codon optimized RdpA mutant L82H |
| SEQ ID NO: 147 | Soybean codon optimized RdpA mutant L82H + V187L |
| SEQ ID NO: 148 | Soybean codon optimized RdpA mutant L82H + V187M |
| SEQ ID NO: 149 | Soybean codon optimized RdpA mutant L82H + V187I |
| SEQ ID NO: 150 | Soybean codon optimized RdpA mutant L82H + V187L + R104A |
| SEQ ID NO: 151 | Soybean codon optimized RdpA mutant L82H + V187L + R104D |
| SEQ ID NO: 152 | Soybean codon optimized RdpA mutant L82H + V187L + R104L |
| SEQ ID NO: 153 | Soybean codon optimized RdpA mutant L82H + V187L + F182W |
| SEQ ID NO: 154 | Soybean codon optimized RdpA mutant L82H + V187L + G103L |
| SEQ ID NO: 155 | Soybean codon optimized RdpA mutant L82H + V187L + F182W + R104G |
| SEQ ID NO: 156 | Soybean codon optimized RdpA mutant L82H + V187L + F182W + G103L |
| SEQ ID NO: 157 | Soybean codon optimized RdpA mutant L82H + V187L + F182W + R104G + T112S |
| SEQ ID NO: 158 | Soybean codon optimized RdpA mutant L82H + V187L + F182W + R104G + V80T |
| SEQ ID NO: 159 | Soybean codon optimized RdpA mutant L82H + V187L + F182W + R104G + R180W |
| SEQ ID NO: 160 | Soybean codon optimized RdpA mutant L82H + V187L + F182W + R104G + R180M |
| SEQ ID NO: 161 | Soybean codon optimized RdpA mutant L82H + V187L + F182W + R104G + D108C |
| SEQ ID NO: 162 | Soybean codon optimized RdpA mutant L82H + V187L + F182W + R104G + D109E |
| SEQ ID NO: 163 | Soybean codon optimized RdpA mutant L82H + V187L + F182W + R104G + Q219C |
| SEQ ID NO: 164 | Soybean codon optimized RdpA mutant L82H + V187L + F182W + R104G + Q219P |
| SEQ ID NO: 165 | Soybean codon optimized RdpA mutant L82H + V187L + F182W + G103L + R180D |
| SEQ ID NO: 166 | Soybean codon optimized RdpA mutant L82H + V187L + F182W + G103L + R180E |
| SEQ ID NO: 167 | Soybean codon optimized RdpA mutant L82H + V187L + F182W + G103L + R180S |
| SEQ ID NO: 168 | Soybean codon optimized RdpA mutant L82H + V187L + F182W + G103L + R180L |
| SEQ ID NO: 169 | Soybean codon optimized RdpA mutant L82H + V187L + F182W + G103L + R180W |
| SEQ ID NO: 170 | Soybean codon optimized RdpA mutant L82H + V187L + F182W + G103L + V80T |
| SEQ ID NO: 171 | Soybean codon optimized RdpA mutant L82H + V187L + F182W + G103L + T112A |
| SEQ ID NO: 172 | Soybean codon optimized RdpA mutant L82H + V187L + F182W + G103L + T112S |
| SEQ ID NO: 173 | Soybean codon optimized RdpA mutant L82H + V187L + F182W + G103L + T112M |
| SEQ ID NO: 174 | Soybean codon optimized RdpA mutant L82H + V187L + F182W + G103L + F247Y |
| SEQ ID NO: 175 | Soybean codon optimized RdpA mutant L82H + V187L + F182W + R104G + V77I |
| SEQ ID NO: 176 | Soybean codon optimized RdpA mutant L82H + V187L + F182W + G103L + R104G + V105Y |
| SEQ ID NO: 177 | Soybean codon optimized RdpA mutant L82H + V187L + F182W + R104G + T112S + R180K |
| SEQ ID NO: 178 | Soybean codon optimized RdpA mutant L82H + V187L + F182W + R104G + T112S + R180M |
| SEQ ID NO: 179 | Soybean codon optimized RdpA mutant L82H + V187L + F182W + R104G + T112S + R180W |
| SEQ ID NO: 180 | Soybean codon optimized RdpA mutant L82H + V187L + F182W + G103L + R180T |
| SEQ ID NO: 181 | Soybean codon optimized RdpA mutant L82H + V187L + F182W + R104G + T112S + R180Q |

### DETAILED DESCRIPTION OF THE INVENTION

The following definitions and methods are provided to better define the invention and to guide those of ordinary skill in the art in the practice of the invention. Unless otherwise noted, terms are to be understood according to conventional usage by those of ordinary skill in the relevant art.

### Engineered Proteins and Recombinant DNA Molecules

The present invention provides novel engineered proteins and the recombinant DNA molecules that encode them. As used herein, the term “engineered” refers to a non-natural DNA, protein or organism that would not normally be found in nature and is created by human intervention. An “engineered protein” is a protein, of which the polypeptide sequence is conceived of and created in laboratory using one or more of the techniques of protein engineering, such as protein design using site-directed mutagenesis and directed evolution using random mutagenesis and DNA shuffling. For example, an engineered protein may have one or more deletion(s), insertion(s) or substitution(s) compared with the coding sequence of the wild-type protein, and each deletion, insertion or substitution may consist of one or more amino acid(s). Examples of engineered proteins are provided herein as SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142.

Engineered proteins provided by the present invention are enzymes with oxygenase activity. As used herein, the term “oxygenase activity” refers to the ability to oxidize a substrate by transferring the oxygen from molecular oxygen to the substrate, byproduct or an intermediate. The oxygenase activity of the engineered proteins provided by the present invention may inactivate one or more of pyridinyloxy acid herbicides.

As used herein, “wild-type” means naturally occurring. As used herein, a “wild-type DNA molecule”, “wild-type polypeptide” or “wild-type protein” is a naturally occurring DNA molecule, polypeptide or protein, i.e., a DNA molecule, polypeptide or protein pre-existing in nature. A wild-type pattern of a polypeptide, protein or DNA molecule may be used for comparison with an engineered protein or gene. A wild-type pattern of a protein or DNA molecule may be used as a control in an experiment.

As used herein, a “control” means an experimental control designed for comparison purposes. For example, a control plant in a transgenic plant analysis is a plant of the same type as the experimental plant (i.e., the plant to be tested) but does not contain the transgenic insert, recombinant DNA molecule, or DNA construct of the experimental plant. An example of a control plant useful for comparison with transgenic maize plants is the non-transgenic LH244 maize (U.S. Pat. No. 6,252,148), and with transgenic soy plants is the non-transgenic A3555 soybean (U.S. Pat. No. 7,700,846).

As used herein, the term “recombinant” refers to a non-natural DNA, polypeptide or protein that is the result of genetic engineering and as such would not normally be found in nature and is created by human intervention. A “recombinant DNA molecule” is a DNA molecule comprising a DNA sequence that does not naturally occur and as such is the result of human intervention, for example, a DNA molecule that encodes an engineered protein. Another example is a DNA molecule comprised of a combination of at least two DNA molecules heterologous to each other, such as a protein-coding DNA molecule and an operably linked heterologous promoter. An example of a recombinant DNA molecule is a DNA molecule comprising at least one sequence selected from SEQ ID NO: 3, 4, 5, 7, 8, 9, 11, 12, 13, 15, 16, 17, 19, 20, 21, 23, 24, 25, 27, 28, 29, 31, 32, 33, 35, 36, 37, 39, 40, 41, 43, 44, 45, 47, 48, 49, 51, 52, 53, 55, 56, 57, 59, 60, 61, 63, 64, 65, 67, 68, 69, 71, 72, 73, 75, 76, 77, 79, 80, 81, 83, 84, 85, 87, 88, 89, 91, 92, 93, 95, 96, 97, 99, 100, 101, 103, 104, 105, 107, 108, 109, 111, 112, 113, 115, 116, 117, 119, 120, 121, 123, 124, 125, 127, 128, 129, 131, 132, 133, 135, 136, 137, 139, 140, 141 and 143-181. A “recombinant polypeptide” or “recombinant protein” is a polypeptide or protein comprising an amino acid sequence that does not naturally occur and as such is the result of human intervention, for example, an engineered protein.

The term “transgene” refers to a DNA molecule artificially incorporated into the genome of an organism as a result of human intervention, such as by plant transformation methods. As used herein, the term “transgenic” means comprising a transgene, for example, a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. Transgenic plants of the present invention comprise the recombinant DNA molecules and engineered proteins provided by the invention.

As used herein, the term “heterologous” refers to the relationship between two or more things derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell or organism into which it is inserted when it would not naturally occur in that particular cell or organism.

As used herein, the term “protein-coding DNA molecule” or “polypeptide-coding DNA molecule” refers to a DNA molecule comprising a nucleotide sequence that encodes a protein or polypeptide. A “protein-coding sequence” or “polypeptide-coding sequence” means a DNA sequence that encodes a protein or polypeptide. A “sequence” means a sequential arrangement of nucleotides or amino acids. The boundaries of a protein-coding sequence or polypeptide-coding sequence are usually determined by a translation start codon at the 5′-terminus and a translation stop codon at the 3′-terminus. A protein-coding molecule or polypeptide-coding molecule may comprise a DNA sequence encoding a protein or polypeptide sequence. As used herein, “transgene expression”, “expressing a transgene”, “protein expression”, “polypeptide expression”, “expressing a protein”, and “expressing a polypeptide” mean the production of a protein or polypeptide through the process of transcribing a DNA molecule into messenger RNA (mRNA) and translating the mRNA into polypeptide chains, which may be ultimately folded into proteins. A protein-coding DNA molecule or polypeptide-coding DNA molecule may be operably linked to a heterologous promoter in a DNA construct for use in expressing the protein or polypeptide in a cell transformed with the recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a protein-coding DNA molecule or polypeptide-coding DNA molecule in a DNA construct where the two DNA molecules are so arranged that the promoter may affect the expression of the transgene.

As used herein, a “DNA construct” is a recombinant DNA molecule comprising two or more heterologous DNA sequences. DNA constructs are useful for transgene expression and may be comprised in vectors and plasmids. DNA constructs may be used in vectors for the purpose of transformation, that is the introduction of heterologous DNA into a host cell, in order to produce transgenic plants and cells, and as such may also be contained in the plasmid DNA or genomic DNA of a transgenic plant, seed, cell or plant part. As used herein, a “vector” means any recombinant DNA molecule that may be used for the purpose of plant transformation. Recombinant DNA molecules as set forth in the sequence listing can, for example, be inserted into a vector as part of a construct having the recombinant DNA molecule operably linked to a promoter that functions in a plant to drive expression of the engineered protein encoded by the recombinant DNA molecule. Methods for constructing DNA constructs and vectors are well known in the art. The components for a DNA construct, or a vector comprising a DNA construct, generally include, but are not limited to, one or more of the following: a suitable promoter for the expression of an operably linked DNA, an operably linked non-human protein-coding DNA molecule, and a 3′ untranslated region (3′-UTR). Promoters useful in practicing the present invention include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional optional components include, but are not limited to, one or more of the following elements: 5′-UTR, enhancer, leader sequence, cis-acting element, intron, chloroplast transit peptides (CTP), and one or more selectable marker transgene(s).

The DNA constructs of the invention may include a CTP molecule operably linked to the protein-coding DNA molecules provided by the invention. A CTP useful in practicing the present invention includes those that function to facilitate localization of the engineered protein molecule within the cell. By facilitating protein localization within the cell, the CTP may increase the accumulation of engineered protein, protect it from proteolytic degradation, enhance the level of herbicide tolerance, and thereby reduce levels of injury after herbicide application. CTP molecules used in the present invention are known in the art and include, but are not limited to the Arabidopsis thaliana EPSPS CTP (Klee et al., 1987), the Petunia hybrida EPSPS CTP (della-Cioppa et al., 1986), the maize cab-m7 signal sequence (Becker et al., 1992; PCT WO 97/41228) and the pea glutathione reductase signal sequence (Creissen et al., 1991; PCT WO 97/41228).

Recombinant DNA molecules of the present invention may be synthesized and modified by methods known in the art, either completely or in part, especially where it is desirable to provide sequences useful for DNA manipulation (such as restriction enzyme recognition sites or recombination-based cloning sites), plant-preferred sequences (such as plant-codon usage or Kozak consensus sequences), or sequences useful for DNA construct design (such as spacer or linker sequences). The present invention includes recombinant DNA molecules and engineered proteins having at least about 80% (percent) sequence identity, about 85% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, and about 99% sequence identity to any of the recombinant DNA molecule or engineered protein sequences provided herein, for instance, to a recombinant DNA molecule comprising a sequence selected from the group consisting of SEQ ID NO: 3, 4, 5, 7, 8, 9, 11, 12, 13, 15, 16, 17, 19, 20, 21, 23, 24, 25, 27, 28, 29, 31, 32, 33, 35, 36, 37, 39, 40, 41, 43, 44, 45, 47, 48, 49, 51, 52, 53, 55, 56, 57, 59, 60, 61, 63, 64, 65, 67, 68, 69, 71, 72, 73, 75, 76, 77, 79, 80, 81, 83, 84, 85, 87, 88, 89, 91, 92, 93, 95, 96, 97, 99, 100, 101, 103, 104, 105, 107, 108, 109, 111, 112, 113, 115, 116, 117, 119, 120, 121, 123, 124, 125, 127, 128, 129, 131, 132, 133, 135, 136, 137, 139, 140, 141 and 143-181. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the GCG® Wisconsin Package® (Accelrys Inc., San Diego, CA), MEGAlign (DNAStar, Inc., 1228S. Park St., Madison, Wis. 53715), and MUSCLE (version 3.6) (RCEdgar, Nucleic Acids Research (2004) 32 (5): 1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequence(s) may be to a full-length sequence or a portion thereof, or to a longer sequence.

Engineered proteins may be produced by changing (i.e., modifying) a wild-type protein to produce a new protein with a novel combination of useful protein characteristics, such as altered Vₘₐₓ, Kₘ, substrate specificity, substrate selectivity, and protein stability. Modifications may be made at specific amino acid positions in a protein and may be a substitution of the amino acid found at that position in nature (i.e., in the wild-type protein) with a different amino acid. The amino acid sequence of the wild-type RdpA protein useful for protein engineering is shown in SEQ ID NO: 1. An engineered protein can be designed that has at least about 92% sequence identity to an amino acid sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142, and comprises at least one of these amino acid mutations. Engineered proteins provided by the invention thus provide a new protein with one or more altered protein characteristic(s) relative to the wild-type protein found in nature. In one embodiment of the present invention, an engineered protein has altered protein characteristics such as improved or decreased activity against one or more herbicide(s) or improved protein stability as compared to a similar wild-type protein or any combination of such characteristics. In one embodiment, the present invention provides an engineered protein and the recombinant DNA molecule encoding it, having at least about 80% sequence identity, about 85% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, and about 99% sequence identity to an engineered protein sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142. Amino acid mutations may be made as a single amino acid substitution in the protein or in combination with one or more other mutation(s), such as one or more other amino acid substitution(s), deletion(s), or addition(s). Mutations may be made as described herein or by any other method known to those of skill in the art.

### Transgenic Plants

An aspect of the invention includes transgenic plant cells, transgenic plant tissues, transgenic plants, and transgenic seeds that comprise the recombinant DNA molecules and engineered proteins provided by the invention. These cells, tissues, plants, and seeds comprising the recombinant DNA molecules and engineered proteins exhibit herbicide tolerance to one or more of pyridinyloxy acid herbicides.

Suitable methods for transforming host plant cells for use in the present invention include virtually any method by which DNA can be introduced into a cell (for example, where a recombinant DNA construct is stably integrated into a plant chromosome) and are well known in the art. An exemplary and widely used method for introducing a recombinant DNA construct into a plant is the Agrobacterium transformation system, which is well known to those of skill in the art. A transgenic plant can be regenerated from a transformed plant cell by the methods of plant cell culture. A transgenic plant homozygous with respect to a transgene (i.e., two allelic copies of the transgene) can be obtained by self-pollinating (selfing) a transgenic plant that contains a single transgene allele with itself, for example an R0 plant, to produce R1 seed. One fourth of the R1 seed produced will be homozygous with respect to the transgene. Plants grown from germinating R1 seed can be tested for zygosity, typically using a SNP assay, DNA sequencing, or a thermal amplification assay that allows for the distinction between heterozygotes and homozygotes, referred to as a zygosity assay.

Plants, seeds, plant parts, plant tissues and cells provided by the present invention exhibit herbicide tolerance to one or more of pyridinyloxy acid herbicides. The pyridinyloxy acid herbicides are synthetic auxins similar to the plant growth hormone indoleacetic acid (IAA). Broad-leaf plants are sensitive to these herbicides, which induce rapid and uncontrolled growth, eventually killing the plant.

Examples of pyridinyloxy acid herbicides include, but are not limited to, the chemical compounds as shown in Formula I, and salt and ester derivatives thereof,

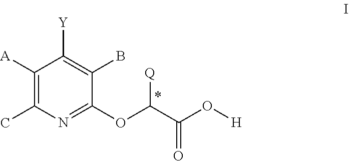

wherein, A and B independently represent halogen, C1-C6 alkyl, halo C1-C6 alkyl, C3-C6 cycloalkyl;
C represents hydrogen, halogen, C1-C6 alkyl, halo C1-C6 alkyl;
Q represents C1-C6 alkyl, halo C1-C6 alkyl, C3-C6 cycloalkyl, C2-C6 alkenyl, C2-C6 alkynyl, halogen, cyano, amino, nitro, formyl, C1-C6 alkoxy, C1-C6 alkylthio, C1-C6 alkoxycarbonyl, hydroxyl C1-C6 alkyl, C1-C6 alkoxy C1-C2 alkyl, cyano C1-C2 alkyl, C1-C6 alkylamino C1-C2 alkyl, benzyl, naphthyl, furyl, thienyl, thiazolyl, pyridinyl, pyrimidinyl, and

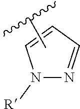

that is unsubstituted or substituted by C1-C6 alkyl, phenyl that is unsubstituted or substituted by at least one group of C1-C6 alkyl, halo C1-C6 alkyl, halogen and C1-C6 alkoxy;
Y represents amino, C1-C6 alkylamino, C1-C6 alkylcarbonylamino, phenylcarbonylamino, benzylamino, furylmethyleneamino that is unsubstituted or substituted by halo C1-C6 alkyl;

The salts are metal salt, ammonium salt NH₄⁺, primary amine salt RNH₂⁺, secondary amine salt (R)₂NH⁺, tertiary ammonium salt (R)₃N⁺, quaternary ammonium salt (R)₄N⁺, morpholine salt, piperidine salt, pyridine salt, aminopropylmorpholine salt, Jeff amine D-230 salt, the salt of 2,4,6-tris (dimethylaminomethyl) phenol and sodium hydroxide, C1-C14 alkyl sulfonium salt, C1-C14 alkyl sulfoxonium salt, C1-C14 alkyl phosphonium salt, C1-C14 alkanol phosphonium salt;
wherein, R independently represents unsubstituted C1-C14 alkyl, C2-C12 alkenyl, C2-C12 alkynyl, C3-C12 cycloalkyl or phenyl, and C1-C14 alkyl optionally substituted by any one or more of the following group(s): halogen, hydroxyl, C1-C6 alkoxy, C1-C6 alkylthio, hydroxyl C1-C6 alkoxy, amino, C1-C6 alkylamino, amino C1-C6 alkylamino, phenyl;

The ester is

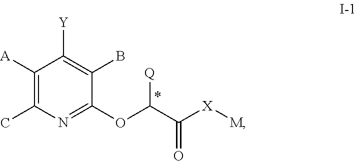

wherein, X represents O or S;
M represents C1-C18 alkyl, halo C1-C8 alkyl, C3-C6 cycloalkyl, C2-C6 alkenyl, halo C2-C6 alkenyl, C2-C6 alkynyl, C1-C6 alkoxy, C1-C6 alkoxycarbonyl, C1-C6 alkylsulfonyl, cyano C1-C2 alkyl, nitro C1-C2 alkyl, C1-C6 alkoxy C1-C2 alkyl, C1-C6 alkoxycarbonyl C1-C2 alkyl, C2-C6 allyloxycarbonyl C1-C2 alkyl, —(C1-C2 alkyl)-Z,

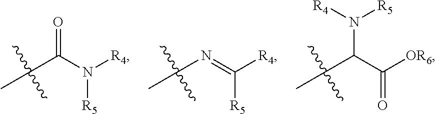

tetrahydrofuranyl, pyridinyl, naphthyl, furyl, thienyl,

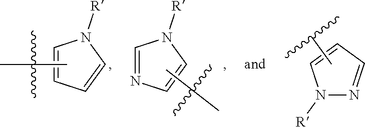

that is unsubstituted or substituted by C1-C6 alkyl, phenyl that is unsubstituted or substituted by C1-C6 alkyl, halo C1-C6 alkyl, C1-C6 alkylamino, halogen or C1-C6 alkoxy;
Z represents

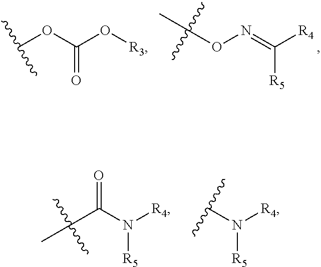

tetrahydrofuranyl, pyridinyl,

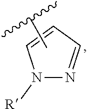

thienyl, furyl, naphthyl, and phenyl that is unsubstituted or substituted by at least one group of C1-C6 alkyl, C1-C6 alkoxy, halo C1-C6 alkyl, cyano and halogen;
R₃ independently represents C1-C6 alkyl;
R₄, R₅, R₆ independently represent hydrogen, C1-C6 alkyl, C1-C6 alkoxycarbonyl;
R′ represents hydrogen, C1-C6 alkyl, halo C1-C6 alkyl.

In one embodiment, the compounds of general formula I and I-1 are both in R configuration (the carbon atoms at * are chiral centers). In another embodiment, in the compound of general formula I, A represents chlorine, B represents chlorine, C represents fluorine, Y represents amino, Q represents methyl, and the compound is in R configuration (the carbon atom at * is the chiral center) (i.e., compound A); in the compound of general formula I-1, A represents chlorine, B represents chlorine, C represents fluorine, Y represents amino, Q represents methyl, X represents O, M represents methyl, and the compound is in R configuration (the carbon atom at * is the chiral center) (i.e., compound B); or in the compound of general formula I-1, A represents chlorine, B represents chlorine, C represents fluorine, Y represents amino, Q represents methyl, X represents O, M represents (tetrahydrofuran-2-yl)methyl

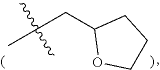

and the compound is in R configuration (i.e., compound C) (the carbon atom at * is the chiral center).

Herbicides may be applied to a plant growing area comprising the plants and seeds provided by the invention as a method for controlling weeds. Plants and seeds provided by the invention comprise a herbicide tolerance trait and as such are tolerant to the application of one or more pyridinyloxyl acid herbicide(s). The plant growing area may or may not comprise weed plants at the time of herbicide application.

The herbicide application may be sequentially or tank mixed with one, two, or a combination of several pyridinyloxy acid herbicides, or any other compatible herbicide. Multiple applications of one herbicide or of two or more herbicides, in combination or alone, may be used over a growing season to an area comprising transgenic plants of the invention to control a broad spectrum of dicot weeds, monocot weeds, or both, for example, two applications (such as a pre-planting application and a post-emergence application, or a pre-emergence application and a post-emergence application) or three applications (such as a pre-planting application, a pre-emergence application, and a post-emergence application, or a pre-emergence application and two post-emergence applications).

As used herein, “tolerance” or “herbicide tolerance” means a plant, seed, plant tissue, plant part or cell's ability to resist the toxic effects of one or more herbicide(s). The herbicide tolerance of a plant, seed, plant tissue, plant part or cell may be measured by comparing the plant, seed, plant tissue, plant part or cell to a suitable control. For example, the herbicide tolerance may be measured by applying a herbicide to a plant comprising a recombinant DNA molecule encoding a protein capable of conferring herbicide tolerance (the test plant) and a plant not comprising the recombinant DNA molecule encoding the protein capable of conferring herbicide tolerance (the control plant) and then comparing the plant injury of the two plants, wherein herbicide tolerance of the test plant is indicated by a reduced injury rate as compared to the injury rate of the control plant. A herbicide-tolerant plant, seed, plant tissue, plant part or cell exhibits a decreased response to the toxic effects of a herbicide when compared to a control plant, seed, plant tissue, plant part or cell. As used herein, an “herbicide tolerance trait” is a transgenic trait imparting improved herbicide tolerance to a plant as compared to a wild-type plant or control plant.

The transgenic plants, progeny, seeds, plant cells, and plant parts of the invention may also contain one or more additional transgenic trait(s). Additional transgenic traits may be introduced by crossing a plant containing a transgene comprising the recombinant DNA molecules provided by the invention with another plant containing an additional transgenic trait(s). As used herein, “crossing” means breeding two individual plants to produce a progeny plant. Two transgenic plants may thus be crossed to produce a progeny that contains the transgenic traits. As used herein, “progeny” means the offspring of any generation of a parent plant, and transgenic progeny comprises a DNA construct provided by the invention and inherited from at least one parent plant. Alternatively, the additional transgenic trait may be introduced by co-transforming the DNA constructs of the additional transgenic trait with a DNA construct comprising a recombinant DNA molecules provided by the invention (e.g., wherein all of the DNA constructs present as part of the same vector used for plant transformation) or by inserting the additional trait into a transgenic plant comprising a DNA construct provided by the invention or vice versa (for example, by using any of the methods of plant transformation on a transgenic plant or plant cell). Such additional transgenic traits include, but are not limited to, increased insect resistance, increased water use efficiency, increased yield performance, increased drought resistance, increased seed quality, improved nutritional quality, hybrid seed production, and herbicide tolerance, in which the trait is measured with respect to a wild-type plant or control plant. Such additional transgenic traits are known to one skilled in the art; for example, a list of such traits is provided by Animal and Plant Health Inspection Service (APHIS), the United States Department of Agriculture (USDA) and can be found on their website at www.aphis.usda.gov.

Transgenic plants and progeny that contain a transgenic trait provided by the invention may be used with any breeding methods that are commonly known in the art. In plant lines comprising two or more transgenic traits, the transgenic traits may be independently segregating, linked, or a combination of both in plant lines comprising three or more transgenic traits. Back-crossing to a parent plant and out-crossing with a non-transgenic plant as well as vegetative propagation are also contemplated. Descriptions of breeding methods that are commonly used for different traits and crops are well known to those of skill in the art. To confirm the presence of the transgene(s) in a particular plant or seed, a variety of assays may be performed. Such assays include, for example, molecular biology assays, such as Southern and Northern blotting, PCR and DNA sequencing; biochemical assays, such as detecting the presence of a protein product, for example, by immunological means (ELISAs and Western blotting) or by enzymatic function; plant part assays, such as leaf or root assays; and also, by analyzing the phenotype of a whole plant.

Introgression of a transgenic trait into a plant genotype is achieved as the result of the process of backcross conversion. A plant genotype into which a transgenic trait that has been introgressed may be referred to as a backcross converted genotype, line, inbred, or hybrid. Similarly, a plant genotype lacking the desired transgenic trait may be referred to as an unconverted genotype, line, inbred, or hybrid.

As used herein, the term “comprising” means “including but not limited to”.

The following embodiments are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein with the same or similar result achieved. All such similar substitutes and modifications apparent to those skilled in the art are deemed to be within the spirit, scope, and concept of the invention as defined by the appended claims.

### Example 1

### Initial Protein Engineering and Enzyme Analysis

By performing homology modeling, molecular docking of RdpA protein, and combining sequence alignment results, the candidate sites were mutated using techniques known to those of skill in the art, such as Alanine-Scanning Mutations, Homology-Scanning Mutations, Pro/Gly Scanning Mutations, Region Swaps or Mutations, and combinations of these various techniques (See M Lehmann and M Wyss, Current Opinion in Biotechnology (2001) 12 (4): 371-375; B Van den Burg and VGH Eijsink, Current Opinion in Biotechnology (2002) 13 (4): 333-337; and Weiss et al., Proc Natl Acad Sci USA (2000) 97 (16): 8950-8954).

The high-throughput bacterial protein expression was achieved by cloning the synthesized gene that encodes each engineered protein into a bacterial expression vector with a C-terminal histidine tag (His-tag). The vectors were transformed into Escherichia coli (E. coli), and expression of the engineered proteins was induced. E. coli cultures were selected and cultured in centrifuge tubes overnight, meanwhile, added with substrates and IPTG, and the cultures were centrifuged to pellet the bacteria the next day. Alternatively, E. coli cultures were selected and cultured in centrifuge tubes overnight, and centrifuged to pellet the bacteria after adding substrates for reaction the next day. The supernatant of reaction solution was pipetted into a 96-well plate and the absorbance at 510 nm from 4-aminoantipyrine and potassium ferricyanide was measured by an endpoint colorimetric method to detect phenol products. Substrate reduction and product yield were measured by high performance liquid chromatography (HPLC) to detect the oxygenase activity (i.e., enzymatic activity) of the engineered proteins, and the activities of the proteins were compared by calculating conversion rates. The results are shown in Table 1.

Conversion rate=(Peak area of initial substrate−Peak area of substrate after reaction)/Peak area of initial substrate*100%

TABLE 1
Conversion rate of each mutant

| | | Conversion | Reaction |
|---|---|---|---|
| Sequence No. | Mutation site | rate | condition |
| SEQ ID NO: 1 | RdpA-wild type | 0.06% | 1 |
| SEQ ID NO: 1 | RdpA-wild type | 0.06% | 2 |
| SEQ ID NO: 2 | L82H | 67.5% | 1 |
| SEQ ID NO: 6 | L82H + V187L | 95.2% | 1 |
| SEQ ID NO: 10 | L82H + V187M | 94.1% | 1 |
| SEQ ID NO: 14 | L82H + V187I | 76.8% | 1 |
| SEQ ID NO: 18 | L82H + V187L + R104A | 92.1% | 1 |
| SEQ ID NO: 22 | L82H + V187L + R104D | 95.5% | 1 |
| SEQ ID NO: 26 | L82H + V187L + R104L | 96.8% | 1 |
| SEQ ID NO: 30 | L82H + V187L + F182W | 96.5% | 1 |
| SEQ ID NO: 34 | L82H + V187L + G103L | 90% | 1 |
| SEQ ID NO: 38 | L82H + V187L + F182W + R104G | 96.9% | 1 |
| SEQ ID NO: 42 | L82H + V187L + F182W + G103L | 96.6% | 1 |
| SEQ ID NO: 46 | L82H + V187L + F182W + R104G + T112S | 97% | 1 |
| SEQ ID NO: 50 | L82H + V187L + F182W + R104G + V80T | 97.2% | 1 |
| SEQ ID NO: 54 | L82H + V187L + F182W + R104G + R180W | 92.5% | 1 |
| SEQ ID NO: 58 | L82H + V187L + F182W + R104G + R180M | 90% | 1 |
| SEQ ID NO: 62 | L82H + V187L + F182W + R104G + D108C | 90% | 1 |
| SEQ ID NO: 66 | L82H + V187L + F182W + R104G + D109E | 94% | 1 |
| SEQ ID NO: 70 | L82H + V187L + F182W + R104G + Q219C | 94% | 1 |
| SEQ ID NO: 74 | L82H + V187L + F182W + R104G + Q219P | 97.2% | 1 |
| SEQ ID NO: 78 | L82H + V187L + F182W + G103L + R180D | 79% | 2 |
| SEQ ID NO: 82 | L82H + V187L + F182W + G103L + R180E | 71.6% | 2 |
| SEQ ID NO: 86 | L82H + V187L + F182W + G103L + R180S | 76.8% | 2 |
| SEQ ID NO: 90 | L82H + V187L + F182W + G103L + R180L | 57.5% | 2 |
| SEQ ID NO: 94 | L82H + V187L + F182W + G103L + R180W | 63.2% | 2 |
| SEQ ID NO: 98 | L82H + V187L + F182W + G103L + V80T | 81% | 2 |
| SEQ ID NO: 102 | L82H + V187L + F182W + G103L + T112A | 93.2% | 2 |
| SEQ ID NO: 106 | L82H + V187L + F182W + G103L + T112S | 86.3% | 2 |
| SEQ ID NO: 110 | L82H + V187L + F182W + G103L + T112M | 54.6% | 2 |
| SEQ ID NO: 114 | L82H + V187L + F182W + G103L + F247Y | 53% | 2 |
| SEQ ID NO: 118 | L82H + V187L + F182W + R104G + V77I | 95.8% | 1 |
| SEQ ID NO: 122 | L82H + V187L + F182W + G103L + R104G + V105Y | 94.3% | 1 |
| SEQ ID NO: 126 | L82H + V187L + F182W + R104G + T112S + R180K | 92.6% | 2 |
| SEQ ID NO: 130 | L82H + V187L + F182W + R104G + T112S + R180M | 88.3% | 2 |
| SEQ ID NO: 134 | L82H + V187L + F182W + R104G + T112S + R180W | 77.6% | 2 |
| SEQ ID NO: 138 | L82H + V187L + F182W + G103L + R180T | 54.6% | 2 |
| SEQ ID NO: 142 | L82H + V187L + F182W + R104G + T112S + R180Q | 67% | 2 |

Note:
Reaction condition 1: The bacteria were cultured overnight, and substrate compound A and IPTG were added to react overnight; Reaction condition 2: The bacteria were cultured overnight, and 8 times dose of substrate compound A in Reaction condition 1 were added to react for 3 hours the next day.

Based on the results of HPLC, representative engineered proteins were selected for protein purification. Further protein characterization, such as Kₘ, Vₘₐₓ and K_{cat}, was performed on the 8 engineered proteins from Table 2. Conventional Ni column affinity chromatography was used for protein purification and protein extract purity was assessed by SDS-PAGE analysis. After determining protein concentration by BCA method, enzyme activity was determined. The controls were purified wild-type enzyme, and the enzymatic kinetic measurements of the proteins were performed using 0, 5, 10, 20, 50, 200, 500 or 1000 μM of compound A. Table 2 shows the Kₘ, Vₘₐₓ, K_{cat} and K_{cat}/Kₘ measured for the 8 proteins with compound A as substrates. The enzymatic kinetic parameters of these 8 engineered proteins demonstrated that the enzymatic activity, i.e., Kₘ and K_{cat}, of the proteins could be significantly improved through protein engineering.

TABLE 2
Determination results of engineered proteins

| | | | Kₘ | K_{cat} | K_{cat}/Kₘ |
|---|---|---|---|---|---|
| Sequence No. | Mutation site | Vₘₐₓ | (μm) | (S⁻¹) | (M⁻¹*S⁻¹*10³) |
| SEQ ID NO: 1 | RdpA-wild type | N/D | | | |
| SEQ ID NO: 38 | L82H + V187L + F182W + R104G | 49.9 | 54.6 | 0.43 | 7.86 |
| SEQ ID NO: 42 | L82H + V187L + F182W + G103L | 99.6 | 81.3 | 0.86 | 10.54 |
| SEQ ID NO: 46 | L82H + V187L + F182W + R104G + T112S | 35.2 | 17.5 | 0.15 | 8.65 |
| SEQ ID NO: 50 | L82H + V187L + F182W + R104G + V80T | 15.7 | 24.9 | 0.07 | 2.71 |
| SEQ ID NO: 54 | L82H + V187L + F182W + R104G + R180W | 81 | 96.9 | 0.35 | 3.59 |
| SEQ ID NO: 58 | L82H + V187L + F182W + R104G + R180M | 12.3 | 28.3 | 0.05 | 1.87 |
| SEQ ID NO: 118 | L82H + V187L + F182W + R104G + V77I | 99.5 | 83.7 | 0.43 | 5.11 |
| SEQ ID NO: 122 | L82H + V187L + F182W + G103L + R104G + V105Y | 127.9 | 96.4 | 1.1 | 11.41 |

Note:
N/D represented the enzymatic activity is too low to determine its enzymatic kinetic parameters.

### Example 2

### Expression of Engineered Proteins in Rice

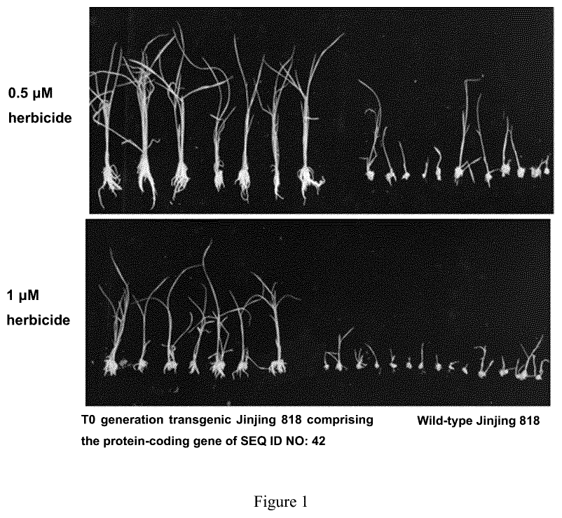

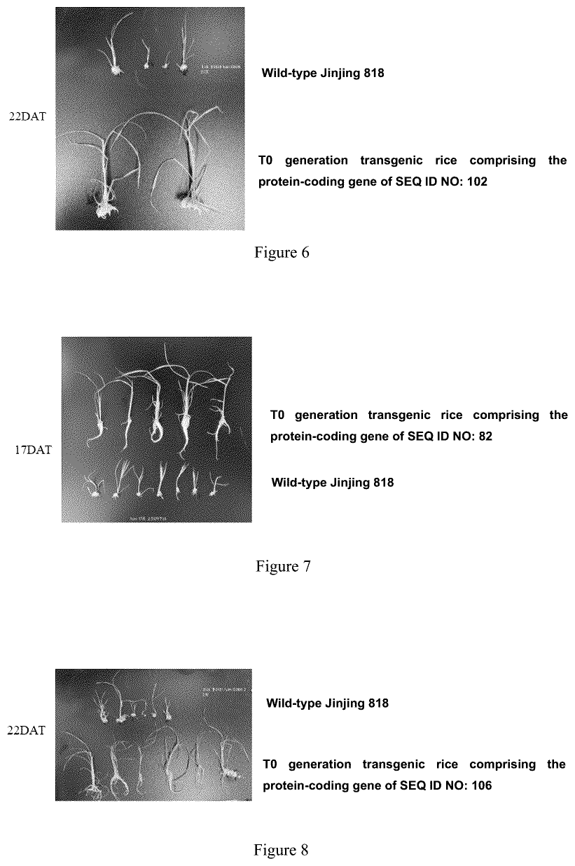

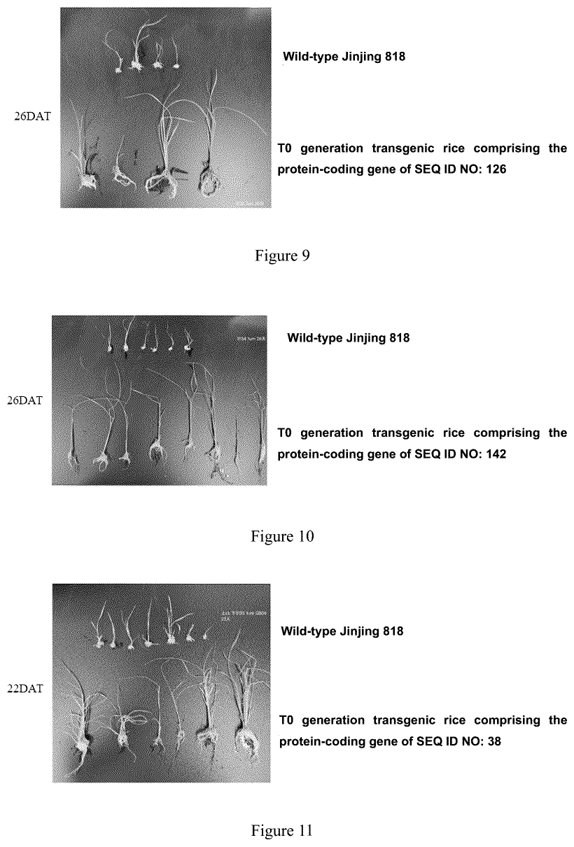

Plant transformation vectors were constructed, each comprising a recombinant DNA molecule encoding an engineered protein (SEQ ID NO: 42/38/82/86/102/106/126/138/142) with the protein-coding sequence optimized for monocot expression, and rice (Jinjing 818) calli were transformed with these vectors using Agrobacterium tumifaciens and standard methods known in the art. Different concentrations of hormone herbicide compound B were added at the rooting phase of the rice seedlings to test hormone resistance, wherein, the test results of control Jinjing 818 plants and transgenic Jinjing 818 plants comprising the protein-coding gene of SEQ ID NO: 42 after adding 0.5 μM and 1 μM compound B to medium for 19 days are shown in FIG. 1. The test results of control Jinjing 818 plants and transgenic Jinjing 818 plants comprising the protein-coding genes of SEQ ID NO: 38/82/86/102/106/126/138/142 after adding 1 μM compound B to medium are shown in FIG. 5-11. Compared with wild-type plants, Jinjing 818 plants comprising the protein-coding gene of SEQ ID NO: 42/38/82/86/102/106/126/138/142 exhibited better drug resistance, which demonstrated that plants expressing the engineered protein exhibited tolerance to compound B herbicide when screened with at least 1 μM of compound B medium.

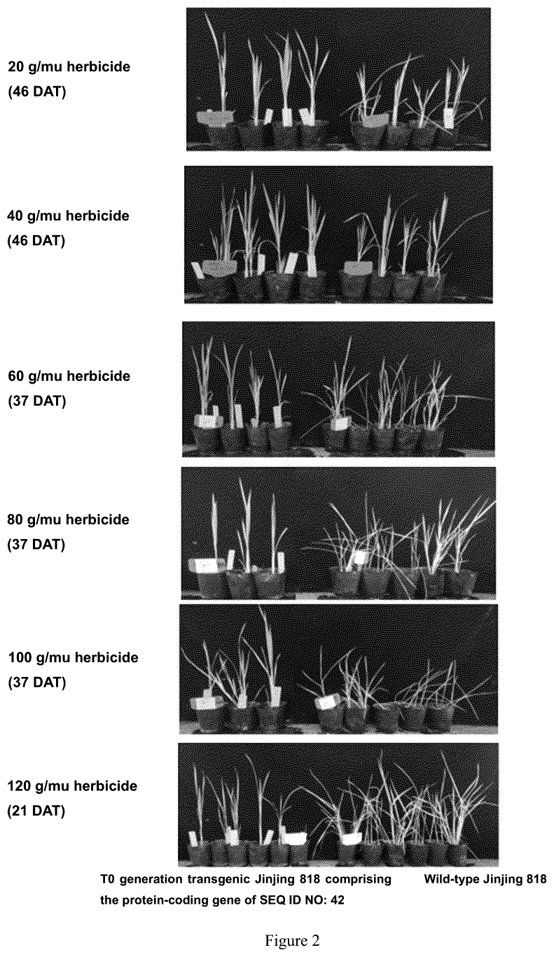

Then, the obtained regenerated TO generation transgenic plantlets grew in the greenhouse, and were sprayed with compound B at the growth stage when approximately two leaves unfolded and the third one just appearing. Resistance levels of plants were recorded and evaluated after spraying treatment, wherein, the test results of control Jinjing 818 plants and transgenic Jinjing 818 plants comprising the protein-coding gene of SEQ ID NO: 42 after applying 20 g, 40 g, 60 g, 80 g, 100 g, 120 g/mu (1 mu=1/15 ha) compound B for corresponding days (DAT) are shown in FIG. 2. Compared with wild-type plants, Jinjing 818 plants comprising the protein-coding gene of SEQ ID NO: 42 exhibited better drug resistance, which demonstrated that plants expressing the engineered protein exhibited tolerance to compound B herbicide when foliage-sprayed with at least 120 g/mu of compound B.

In summary, in the medicated medium test and the greenhouse spraying test for TO generation, the plants comprising the recombinant DNA molecule encoding the engineered protein (SEQ ID NO: 42/38/82/86/102/106/126/138/142) all exhibited apparent tolerance compared with the wild types.

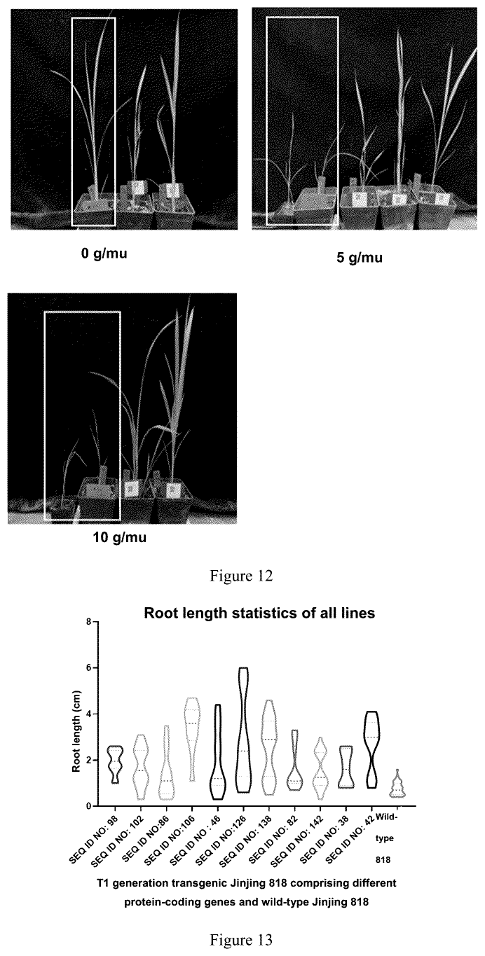

The obtained regenerated TO generation transgenic plantlets grew in the greenhouse, and were sprayed with compound quizalofop-P-ethyl at the growth stage when approximately two leaves unfolded and the third one just appearing. Resistance levels of plants were recorded and evaluated after spraying treatment, wherein, the test results of control Jinjing 818 plants and transgenic Jinjing 818 plants comprising the protein-coding gene of SEQ ID NO: 46 after applying 0 g, 5 g, 10 g/mu compound quizalofop-P-ethyl for 20 DAT are shown in FIG. 12. Compared with wild-type plants, Jinjing 818 plants comprising the protein-coding gene of SEQ ID NO: 46 exhibited better drug resistance, which demonstrated that plants expressing the engineered protein exhibited tolerance to compound quizalofop-P-ethyl herbicide when foliage-sprayed with at least 10 g/mu of compound quizalofop-P-ethyl.

The transformed TO transgenic plants grew in the greenhouse. T1 generation rice plant seeds produced by transformation of all constructs were collected. The resistance level of each construct was tested and compared by adding 0.3 μM compound B to the liquid of T1 generation and soaking seeds. Comparisons were made by measuring the root length of each construct. As shown in FIG. 13, after adding 0.3 μM compound B and soaking seeds for 20 days, T1 generation transgenic Jinjing 818 seeds comprising different protein-coding genes all exhibited better drug resistance compared with wild-type Jinjing 818, presenting as longer roots, which demonstrated that plants expressing engineered proteins exhibited tolerance to compound B herbicide when screened by hydroponics and seed soaking with 0.3 μM compound B.

### Example 3

Expression of Engineered Proteins in Arabidopsis thaliana

Engineered proteins were selected for Arabidopsis thaliana transformation and plant analysis. DNA constructs were transformed into Arabidopsis thaliana using Agrobacterium tumifaciens and standard methods known in the art.

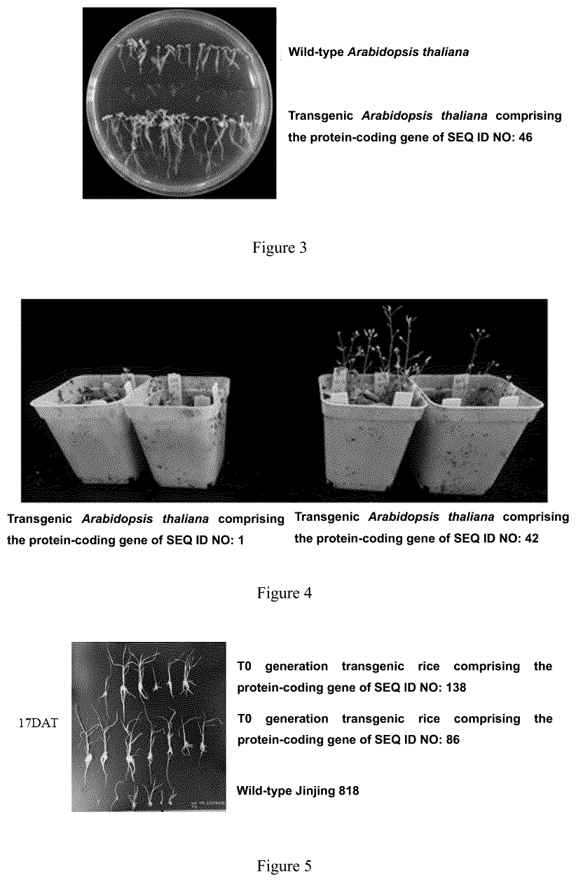

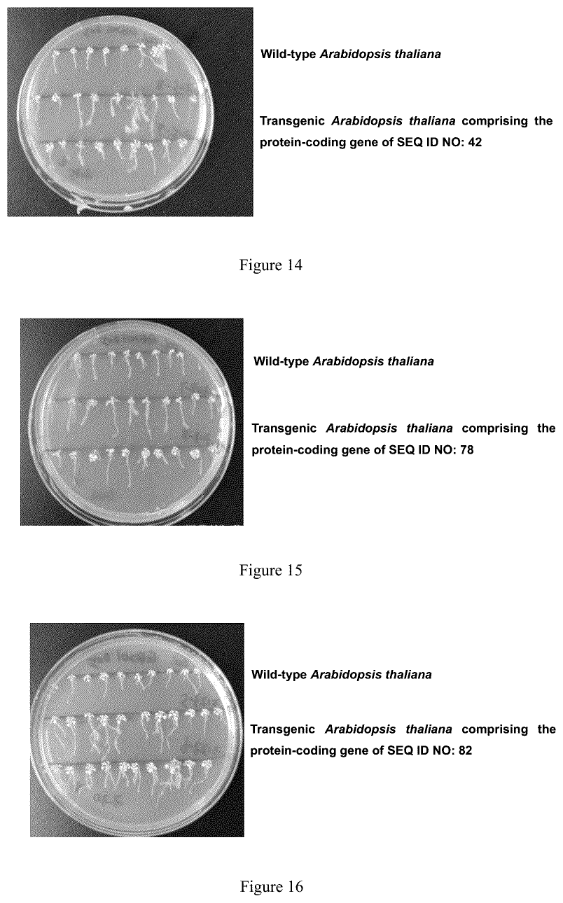

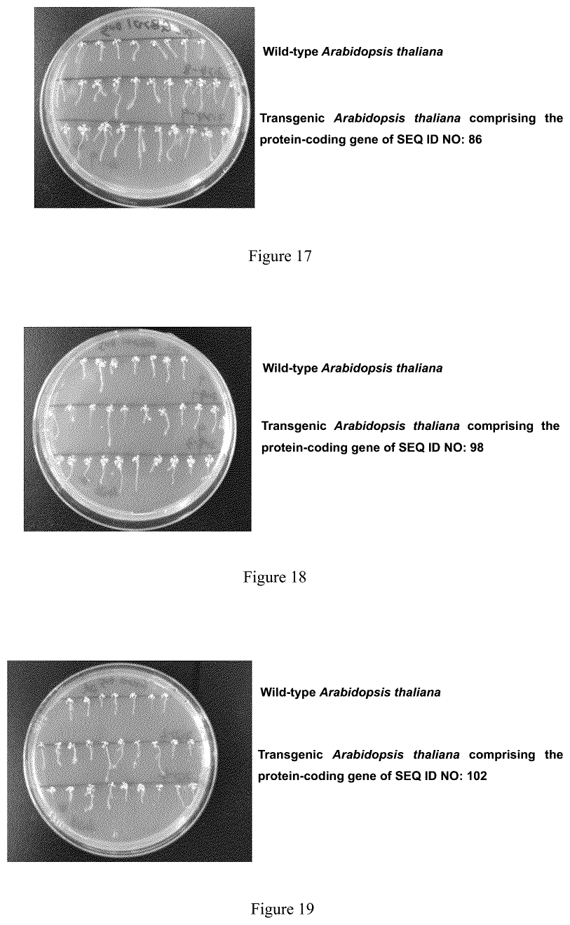

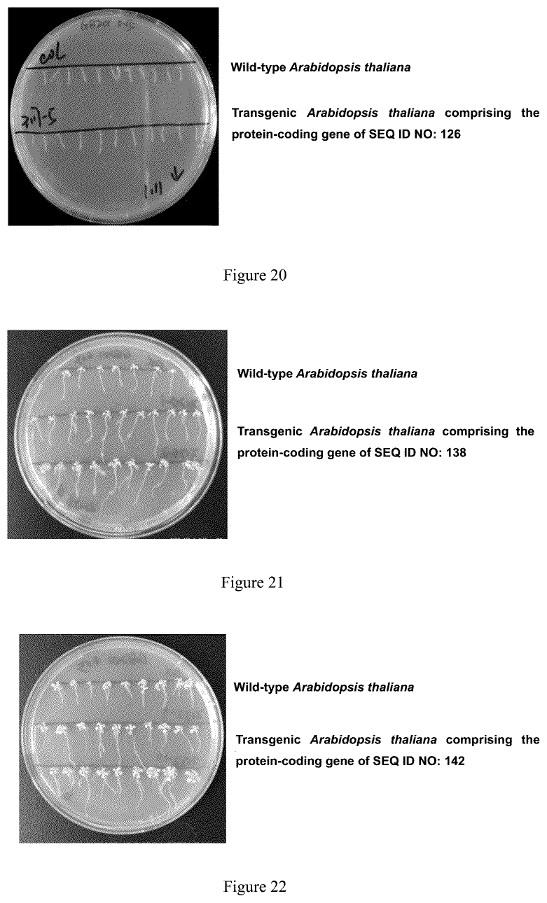

The transformed TO transgenic plantlets grew in the greenhouse. T1 generation Arabidopsis thaliana plant seeds produced by transformation of all constructs were collected after approximately 60 days of growth. Transgenic T1 generation Arabidopsis thaliana plants were screened by adding HYG to medium for T1 generation. T1 plants were selfed to produce seeds of T2 generation Arabidopsis thaliana plants. Wherein, compound A was added to medium to screen and test T2 generation Arabidopsis thaliana plant seeds, that were produced by all constructs, comprising unique events screened by T1 generation. As shown in FIGS. 3, 14-22, after adding 0.15 μM compound A to medium for corresponding days of screening, the transgenic T2 Arabidopsis thaliana seeds comprising the protein-coding gene of SEQ ID NO: 46/42/78/82/86/98/102/126/138/142 exhibited better drug resistance compared with the wild-type Arabidopsis thaliana, presenting as longer roots and larger leaves. It demonstrated that plants expressing the engineered protein exhibited tolerance to compound A herbicide when screened with 0.15 μM of compound A medium.

In addition, compound B was sprayed to test T2 generation Arabidopsis thaliana plants, that were produced by some of the constructs, comprising unique events screened by T1 generation, wherein, the test results of the wild-type Arabidopsis thaliana control plants transformed with RdpA gene and Arabidopsis thaliana plants comprising the protein-coding gene of SEQ ID NO: 42 after being foliage-sprayed with 40 g/mu compound B for 12 days are shown in FIG. 4. Compared with the wild-type plants transformed with RdpA gene, Arabidopsis thaliana plants comprising the protein-coding gene of SEQ ID NO: 42 exhibited better drug resistance, which demonstrated that plants expressing the engineered protein exhibited tolerance to compound B herbicide when foliage-sprayed with 40 g/mu of compound B.

### Example 4

### Expression of Engineered Proteins in Soybeans

Engineered proteins were selected for soybean transformation and plant analysis. DNA constructs were transformed into soybeans using Agrobacterium tumifaciens and standard methods known in the art.

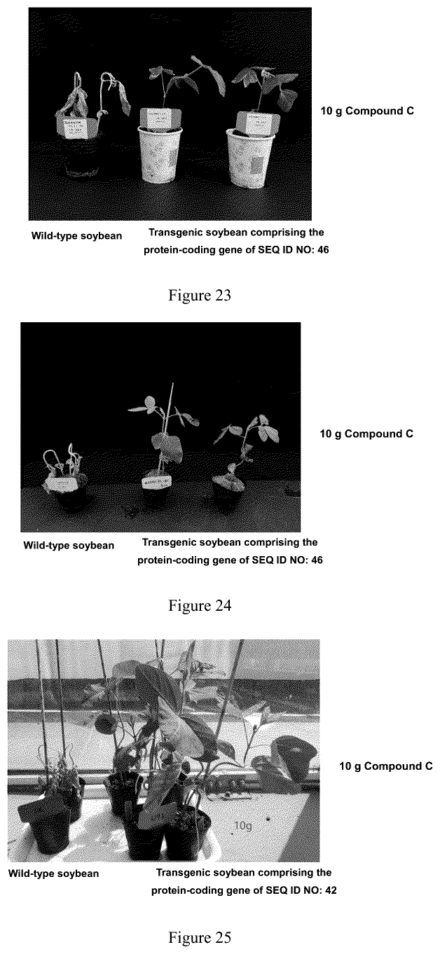

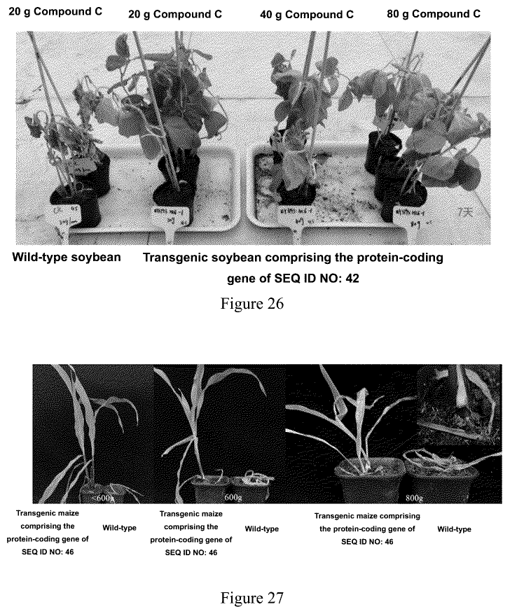

The transformed TO transgenic plantlets grew in the greenhouse. TO generation transgenic plantlets were tested by spraying 10 g compound C. As shown in FIG. 23, the transgenic soybeans comprising the protein-coding gene of SEQ ID NO: 46 exhibited apparent resistance compared with the wild-type soybean. T1 generation soybean plant seeds produced by transformation of all constructs were collected, and T1 generation seedlings were tested by spraying 10 g compound C. As shown in FIG. 24, the transgenic soybeans comprising the protein-coding gene of SEQ ID NO: 46 exhibited apparent resistance compared with the wild-type soybean. T1 generation transgenic plantlets produced by some of the constructs were sprayed with 10 g, 20 g, 40 g, 80 g compound C, and as shown in FIG. 25-26, the transgenic soybeans comprising the protein-coding gene of SEQ ID NO: 42 exhibited better drug resistance compared with the wild-type soybeans when applying at least 40 g compound C.

### Example 5

### Expression of Engineered Proteins in Maizes

Engineered proteins were selected for maize transformation and plant analysis. DNA constructs were transformed into maizes using Agrobacterium tumifaciens and standard methods known in the art.

The transformed TO transgenic plantlets grew in the greenhouse. TO generation transgenic plantlets were tested by spraying with 150 g, 250 g, 400 g, 600 g, 800 g of 30% glyphosate compound C (25+5) ME. As shown in FIG. 27, the wild types treated by each concentration all died, and the transgenic plantlets had no apparent phytotoxicity reaction when treated at a concentration below 600 g (representative image of the data below 600 g as shown on the far left in FIG. 27). At a concentration of 600 g, the stem base was slightly enlarged, the whole plant growth was obviously inhibited, and the plant was in normal condition. At a concentration of 800 g, the stem base was significantly enlarged, inhibition on the whole plant growth was further intensified, and the leaf color became lighter and lusterless. Thus it may be concluded that the transgenic maize comprising the protein-coding gene of SEQ ID NO: 46 had better drug resistance compared with the wild-type maize when applying 600 g 30% glyphosate compound C (25+5) ME.

Meanwhile, it was found after many tests that, the introduction of the recombinant DNA molecule of the present invention into model plants, such as Arabidopsis thaliana, Brachypodium distachyon, etc., resulted in corresponding increases in drug resistance to pyridinyloxy acid herbicides. Thus, it may be known that transforming it into other plants, such as food crops, legume crops, oil crops, fiber crops, fruit crops, root crops, vegetable crops, flower crops, medicine crops, raw material crops, pasture crops, sugar crops, beverage crops, lawn plants, forest crops, nut crops, etc. would also produce corresponding resistance traits, which have good industrial value.

1.-24. (canceled)

25. A recombinant DNA molecule, comprising a nucleic acid sequence encoding a polypeptide, as compared with the amino acid sequence of RdpA as set forth in SEQ ID NO: 1, the amino acid sequence of the polypeptide has the following mutation: the amino acid at position 82 is mutated from leucine into histidine.

26. The recombinant DNA molecule according to claim 25, wherein the amino acid sequence of the polypeptide also has one or more mutation(s) selected from the following groups:
the amino acid at position 187 is mutated from valine into leucine, methionine or isoleucine;
the amino acid at position 187 is mutated from valine into leucine, and the amino acid at position 104 is mutated from arginine into alanine, aspartic acid or leucine;
the amino acid at position 187 is mutated from valine into leucine, and the amino acid at position 182 is mutated from phenylalanine into tryptophan;
the amino acid at position 187 is mutated from valine into leucine, and the amino acid at position 103 is mutated from glycine into leucine;
the amino acid at position 187 is mutated from valine into leucine, the amino acid at position 182 is mutated from phenylalanine into tryptophan, and the amino acid at position 104 is mutated from arginine into glycine;
the amino acid at position 187 is mutated from valine into leucine, the amino acid at position 182 is mutated from phenylalanine into tryptophan, and the amino acid at position 103 is mutated from glycine into leucine;
the amino acid at position 187 is mutated from valine into leucine, the amino acid at position 182 is mutated from phenylalanine into tryptophan, the amino acid at position 104 is mutated from arginine into glycine, and the amino acid at position 112 is mutated from threonine into serine;
the amino acid at position 187 is mutated from valine into leucine, the amino acid at position 182 is mutated from phenylalanine into tryptophan, the amino acid at position 104 is mutated from arginine into glycine, and the amino acid at position 80 is mutated from valine into threonine;
the amino acid at position 187 is mutated from valine into leucine, the amino acid at position 182 is mutated from phenylalanine into tryptophan, the amino acid at position 104 is mutated from arginine into glycine, and the amino acid at position 180 is mutated from arginine into tryptophan or methionine;
the amino acid at position 187 is mutated from valine into leucine, the amino acid at position 182 is mutated from phenylalanine into tryptophan, the amino acid at position 104 is mutated from arginine into glycine, and the amino acid at position 108 is mutated from aspartic acid into cysteine;
the amino acid at position 187 is mutated from valine into leucine, the amino acid at position 182 is mutated from phenylalanine into tryptophan, the amino acid at position 104 is mutated from arginine into glycine, and the amino acid at position 109 is mutated from aspartic acid into glutamic acid;
the amino acid at position 187 is mutated from valine into leucine, the amino acid at position 182 is mutated from phenylalanine into tryptophan, the amino acid at position 104 is mutated from arginine into glycine, and the amino acid at position 219 is mutated from glutamine into cysteine or proline;
the amino acid at position 187 is mutated from valine into leucine, the amino acid at position 182 is mutated from phenylalanine into tryptophan, the amino acid at position 103 is mutated from glycine into leucine, and the amino acid at position 180 is mutated from arginine into aspartic acid, glutamic acid, serine, leucine, tryptophan or threonine;
the amino acid at position 187 is mutated from valine into leucine, the amino acid at position 182 is mutated from phenylalanine into tryptophan, the amino acid at position 103 is mutated from glycine into leucine, and the amino acid at position 80 is mutated from valine into threonine;
the amino acid at position 187 is mutated from valine into leucine, the amino acid at position 182 is mutated from phenylalanine into tryptophan, the amino acid at position 103 is mutated from glycine into leucine, and the amino acid at position 112 is mutated from threonine into alanine, serine or methionine;
the amino acid at position 187 is mutated from valine into leucine, the amino acid at position 182 is mutated from phenylalanine into tryptophan, the amino acid at position 103 is mutated from glycine into leucine, and the amino acid at position 247 is mutated from phenylalanine into tyrosine;
the amino acid at position 187 is mutated from valine into leucine, the amino acid at position 182 is mutated from phenylalanine into tryptophan, the amino acid at position 104 is mutated from arginine into glycine, and the amino acid at position 77 is mutated from valine into isoleucine;
the amino acid at position 187 is mutated from valine into leucine, the amino acid at position 182 is mutated from phenylalanine into tryptophan, the amino acid at position 104 is mutated from arginine into glycine, the amino acid at position 112 is mutated from threonine into serine, and the amino acid at position 180 is mutated from arginine into lysine, methionine, tryptophan or glutamine; and/or
the amino acid at position 187 is mutated from valine into leucine, the amino acid at position 182 is mutated from phenylalanine into tryptophan, the amino acid at position 103 is mutated from glycine into leucine, the amino acid at position 104 is mutated from arginine into glycine, and the amino acid at position 105 is mutated from valine into tyrosine.

27. The recombinant DNA molecule according to claim 25, wherein the recombinant DNA molecule has one or more characteristics selected from the following:
(1) the amino acid sequence of the polypeptide further has at least 80%, at least 85%, at least 90%, at least 95%, at least 96%, at least 97%, at least 98%, at least 99% sequence identity to the amino acid sequence of RdpA as set forth in SEQ ID NO: 1;
(2) the amino acid sequence of the polypeptide has at least 92%, at least 95%, at least 96%, at least 97%, at least 98%, at least 99% or 100% sequence identity to an amino acid sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142;
(3) the nucleic acid sequence is selected from the group consisting of SEQ ID NO: 3, 4, 5, 7, 8, 9, 11, 12, 13, 15, 16, 17, 19, 20, 21, 23, 24, 25, 27, 28, 29, 31, 32, 33, 35, 36, 37, 39, 40, 41, 43, 44, 45, 47, 48, 49, 51, 52, 53, 55, 56, 57, 59, 60, 61, 63, 64, 65, 67, 68, 69, 71, 72, 73, 75, 76, 77, 79, 80, 81, 83, 84, 85, 87, 88, 89, 91, 92, 93, 95, 96, 97, 99, 100, 101, 103, 104, 105, 107, 108, 109, 111, 112, 113, 115, 116, 117, 119, 120, 121, 123, 124, 125, 127, 128, 129, 131, 132, 133, 135, 136, 137, 139, 140, 141 and 143-181, and a nucleic acid sequence encoding the same amino acid sequence as the sequence shown due to degeneracy of genetic code.

28. The recombinant DNA molecule according to claim 25, wherein the recombinant DNA molecule is operably linked to a heterologous promoter functional in a plant cell.

29. The recombinant DNA molecule according to claim 28, wherein the recombinant DNA molecule is further operably linked to a DNA molecule encoding a chloroplast transit peptide.

30. A DNA construct comprising a heterologous promoter functional in a plant cell operably linked to the recombinant DNA molecule according to claim 25.

31. The DNA construct according to claim 30, further comprising a DNA molecule encoding a chloroplast transit peptide operably linked to the recombinant DNA molecule.

32. The DNA construct according to claim 30, wherein the DNA construct exists in the genome of a transgenic plant.

33. A plant, seed, plant tissue, plant part or cell, comprising the recombinant DNA molecule according to claim 30.

34. The plant, seed, plant tissue, plant part or cell according to claim 33, wherein the plant, seed, plant tissue, plant part or cell comprises tolerance to at least one herbicide selected from the group consisting of pyridinyloxy acid herbicides.

35. A plant, seed, plant tissue, plant part or cell, comprising the DNA construct according to claim 30.

36. A plant, seed, plant tissue, plant part or cell, comprising the polypeptide encoded by the recombinant DNA molecule according to claim 25.

37. A polypeptide, of which the amino acid sequence having the following mutation compared with the amino acid sequence of RdpA as set forth in SEQ ID NO: 1: the amino acid at position 82 is mutated from leucine into histidine; optionally, also having one or more mutation(s) selected from the following groups:
the amino acid at position 187 is mutated from valine into leucine, methionine or isoleucine;
the amino acid at position 187 is mutated from valine into leucine, and the amino acid at position 104 is mutated from arginine into alanine, aspartic acid or leucine;
the amino acid at position 187 is mutated from valine into leucine, and the amino acid at position 182 is mutated from phenylalanine into tryptophan;
the amino acid at position 187 is mutated from valine into leucine, and the amino acid at position 103 is mutated from glycine into leucine;
the amino acid at position 187 is mutated from valine into leucine, the amino acid at position 182 is mutated from phenylalanine into tryptophan, and the amino acid at position 104 is mutated from arginine into glycine;
the amino acid at position 187 is mutated from valine into leucine, the amino acid at position 182 is mutated from phenylalanine into tryptophan, and the amino acid at position 103 is mutated from glycine into leucine;
the amino acid at position 187 is mutated from valine into leucine, the amino acid at position 182 is mutated from phenylalanine into tryptophan, the amino acid at position 104 is mutated from arginine into glycine, and the amino acid at position 112 is mutated from threonine into serine;
the amino acid at position 187 is mutated from valine into leucine, the amino acid at position 182 is mutated from phenylalanine into tryptophan, the amino acid at position 104 is mutated from arginine into glycine, and the amino acid at position 80 is mutated from valine into threonine;
the amino acid at position 187 is mutated from valine into leucine, the amino acid at position 182 is mutated from phenylalanine into tryptophan, the amino acid at position 104 is mutated from arginine into glycine, and the amino acid at position 180 is mutated from arginine into tryptophan or methionine;
the amino acid at position 187 is mutated from valine into leucine, the amino acid at position 182 is mutated from phenylalanine into tryptophan, the amino acid at position 104 is mutated from arginine into glycine, and the amino acid at position 108 is mutated from aspartic acid into cysteine;
the amino acid at position 187 is mutated from valine into leucine, the amino acid at position 182 is mutated from phenylalanine into tryptophan, the amino acid at position 104 is mutated from arginine into glycine, and the amino acid at position 109 is mutated from aspartic acid into glutamic acid;
the amino acid at position 187 is mutated from valine into leucine, the amino acid at position 182 is mutated from phenylalanine into tryptophan, the amino acid at position 104 is mutated from arginine into glycine, and the amino acid at position 219 is mutated from glutamine into cysteine or proline;
the amino acid at position 187 is mutated from valine into leucine, the amino acid at position 182 is mutated from phenylalanine into tryptophan, the amino acid at position 103 is mutated from glycine into leucine, and the amino acid at position 180 is mutated from arginine into aspartic acid, glutamic acid, serine, leucine, tryptophan or threonine;
the amino acid at position 187 is mutated from valine into leucine, the amino acid at position 182 is mutated from phenylalanine into tryptophan, the amino acid at position 103 is mutated from glycine into leucine, and the amino acid at position 80 is mutated from valine into threonine;
the amino acid at position 187 is mutated from valine into leucine, the amino acid at position 182 is mutated from phenylalanine into tryptophan, the amino acid at position 103 is mutated from glycine into leucine, and the amino acid at position 112 is mutated from threonine into alanine, serine or methionine;
the amino acid at position 187 is mutated from valine into leucine, the amino acid at position 182 is mutated from phenylalanine into tryptophan, the amino acid at position 103 is mutated from glycine into leucine, and the amino acid at position 247 is mutated from phenylalanine into tyrosine;
the amino acid at position 187 is mutated from valine into leucine, the amino acid at position 182 is mutated from phenylalanine into tryptophan, the amino acid at position 104 is mutated from arginine into glycine, and the amino acid at position 77 is mutated from valine into isoleucine;
the amino acid at position 187 is mutated from valine into leucine, the amino acid at position 182 is mutated from phenylalanine into tryptophan, the amino acid at position 104 is mutated from arginine into glycine, the amino acid at position 112 is mutated from threonine into serine, and the amino acid at position 180 is mutated from arginine into lysine, methionine, tryptophan or glutamine; and/or
the amino acid at position 187 is mutated from valine into leucine, the amino acid at position 182 is mutated from phenylalanine into tryptophan, the amino acid at position 103 is mutated from glycine into leucine, the amino acid at position 104 is mutated from arginine into glycine, and the amino acid at position 105 is mutated from valine into tyrosine.

38. The polypeptide according to claim 37, wherein the polypeptide has one or more characteristics selected from the following:
(1) the amino acid sequence of the polypeptide further has at least 80%, at least 85%, at least 90%, at least 95%, at least 96%, at least 97%, at least 98%, at least 99% sequence identity to the amino acid sequence of RdpA as set forth in SEQ ID NO: 1;
(2) the amino acid sequence of the polypeptide has at least 92%, at least 95%, at least 96%, at least 97%, at least 98%, at least 99% or 100% sequence identity to an amino acid sequence selected from the group consisting of SEQ ID NO: 2, 6, 10, 14, 18, 22, 26, 30, 34, 38, 42, 46, 50, 54, 58, 62, 66, 70, 74, 78, 82, 86, 90, 94, 98, 102, 106, 110, 114, 118, 122, 126, 130, 134, 138 and 142;
(3) the polypeptide has oxygenase activity against at least one herbicide selected from the group consisting of pyridinyloxy acid herbicides.

39. A method for conferring herbicide tolerance to a plant, seed, cell or plant part, comprising expressing the polypeptide according to claim 37, in the plant, seed, cell or plant part.

40. The method according to claim 39, wherein, the plant, seed, cell or plant part comprises a DNA construct, the DNA construct comprises a heterologous promoter functional in a plant cell operably linked to a recombinant DNA molecule, the recombinant DNA molecule comprises the nucleic acid sequence encoding the polypeptide according to claim 37.

41. The method according to claim 37, wherein the plant, seed, cell or plant part comprises tolerance to at least one herbicide selected from the group consisting of pyridinyloxy acid herbicides.

42. A method for producing a herbicide tolerant transgenic plant comprising transforming a plant cell or tissue with the recombinant DNA molecule according to claim 30 or a DNA construct comprising the recombinant DNA molecule, and regenerating a herbicide tolerant transgenic plant from the transformed plant cell or tissue.

43. The method according to claim 42, wherein the herbicide tolerant transgenic plant comprises tolerance to at least one herbicide selected from the group consisting of pyridinyloxy acid herbicides.

44. A method for controlling weeds in a plant growing area, comprising exposing a plant growing area that comprises a plant or seed to at least one herbicide selected from the group consisting of pyridinyloxy acid herbicides, wherein the plant or seed comprises the recombinant DNA molecule according to claim 25 and is tolerant to at least one of the herbicides.


SEQUENCE LISTING (181 sequences)

SEQ ID NO: 1 - protein, 295 aa, Sphingobium herbicidovorans
MHAALSPLSQRFERIAVQPLTGVLGAEITGVDLREPLDDSTWNEILDAFHTYQVIYFPGQAITNEQHIAFSRRFGPVDPVPLLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSVKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 2 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSVKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 3 - DNA, 888 bp, synthetic construct
  source 1..888: mol_type = other DNA
  CDS 1..888: protein_id = 2; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSVKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcgctgtctccgctgtctcagcgctttgaacgtattgcagttcaaccgctgaccggtgttctgggcgctgaaatcaccggcgtggacctgcgtgaaccgctggatgattccacttggaacgaaattctggacgcctttcacacttaccaggttatttattttccgggtcaagcgatcaccaacgaacaacacattgcgtttagccgtcgcttcggcccagtagatccggttccacacctgaaatctatcgaaggttacccggaagttcagatgatccgtcgtgaggctaacgaaagcggccgtgtaatcggcgacgactggcacaccgactctaccttcctggacgcaccgccggctgcagttgttatgcgcgcaatcgatgttccggaacacggtggcgataccggcttcctgtctatgtacaccgcttgggaaactctgtccccaactatgcaggcaaccatcgagggtctgaacgtagttcactctgctacccgtgtgtttggctccctgtaccaagcgcagaaccgtcgcttcagcaacacctccgtgaaggtgatggatgttgatgcgggcgatcgtgaaactgttcacccgctggtagtaacccatccaggctctggccgtaaaggtctgtacgttaaccaagtatactgtcagcgtatcgaaggtatgactgatgcggaatccaaaccgctgctgcagttcctgtacgaacacgcgacgcgcttcgattttacttgccgtgtacgttggaaaaaagatcaggttctggtgtgggataacctgtgcaccatgcaccgtgcagtgccagactacgcgggtaaattccgctacctgacgcgtactactgtgggtggtgtgcgtccggcacgttaa

SEQ ID NO: 4 - DNA, 888 bp, synthetic construct
  source 1..888: mol_type = other DNA
  CDS 1..888: protein_id = 2; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSVKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctttgtctccgttgtcccagaggtttgagcgtatcgccgtccagccactcacaggtgtgctcggtgcagagatcaccggcgtcgatttgcgcgagccactggacgactctacttggaacgagattttggatgcttttcatacctaccaggtgatctacttcccgggtcaagccattaccaacgaacagcatatcgctttctccaggaggttcggcccggtggatcctgtgccgcacttgaagtccatcgagggttaccctgaagtccagatgattcggagagaggccaacgagagcggtagggttattggcgatgactggcatacggacagcaccttcctggatgcgccgccagccgctgttgtgatgagggctatcgacgtccccgagcacgggggcgacactgggttcttgtctatgtatacggcctgggagaccctttccccaaccatgcaggcaactatcgagggcctgaatgtcgtgcacagtgcgaccagagtgtttgggtccttgtaccaggctcaaaacagacgctttagcaatacttcagtcaaggtcatggatgttgatgcgggtgacagggagactgtccaccctcttgtcgttacccatccaggctcaggtaggaagggtctgtacgtcaaccaggtctactgtcagcgcattgaggggatgacagatgcggagtcgaagcctttgctgcaattcctttacgagcacgccacccggttcgatttcacatgtcgggtccgctggaagaaagaccaggtccttgtttgggataatctctgcacaatgcaccgcgctgttcccgactatgccggcaaatttcgttacctcactaggactacagttggcggcgtcaggcccgcccgctga

SEQ ID NO: 5 - DNA, 888 bp, synthetic construct
  source 1..888: mol_type = other DNA
  CDS 1..888: protein_id = 2; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSVKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactttctcctctcagccagaggtttgaaagaattgctgtacaacctttgacaggagtcttgggagccgaaattactggggtagaccttcgagagcccctggatgattctacctggaacgaaattttagacgcatttcatacttaccaagttatttatttcccaggacaagctattaccaacgaacagcacattgcatttagccgaagattcggtcctgttgaccctgtgcctcatttaaaatctatcgaaggttaccccgaggtacaaatgattagaagagaggctaacgagtcaggtcgtgtgatcggggatgattggcacactgatagcacttttttggacgcaccacctgctgctgtcgtcatgcgtgctatcgatgtaccagaacatgggggtgatacagggttcttgagtatgtacactgcttgggagactttgtctcctactatgcaagctacaatcgaaggccttaacgtcgtgcatagtgccaccagggtgttcgggtctctttaccaggcacaaaataggagattttctaatacctcagtaaaggtgatggatgttgacgccggcgatagagaaactgtgcatccacttgtcgttactcacccaggttcagggaggaagggtctgtatgttaatcaagtttactgccaacgaatcgagggtatgactgacgcagagtcaaagcctttgttgcaatttctttatgaacatgcaactagatttgatttcacatgccgtgtgaggtggaagaaagatcaggttcttgtttgggataatttgtgtactatgcatagagctgttcctgactatgctggtaagtttagatacctcaccagaacaaccgttggtggtgtccgtcctgctcgatga

SEQ ID NO: 6 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 7 - DNA, 888 bp, synthetic construct
  source 1..888: mol_type = other DNA
  CDS 1..888: protein_id = 6; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagctctgtctccgctgtcccagcgtttcgaacgtattgctgtacaaccgctgaccggcgttctgggtgctgagattactggcgttgatctgcgtgagccgctggatgactctacctggaacgagatcctggacgcgttccacacctaccaggtcatttacttcccgggtcaggcaatcactaacgaacagcacattgctttctctcgccgttttggtccggtggatccagttccgcacctgaagtctatcgaaggctatccggaagttcagatgattcgccgcgaagctaacgaaagcggccgtgttatcggcgatgactggcataccgactctacctttctggatgcaccgccggccgctgttgttatgcgtgcaatcgacgtaccggaacatggtggtgacaccggctttctgtccatgtataccgcctgggaaaccctgtctccgaccatgcaggcaactattgaaggtctgaacgtcgttcacagcgccactcgcgttttcggctctctgtaccaggctcagaaccgtcgtttcagcaacacctccctgaaggttatggacgttgacgctggcgatcgcgaaaccgtccacccgctggttgttactcacccgggctctggtcgcaaaggtctgtatgttaaccaggtttactgtcagcgtatcgaaggcatgaccgatgcagaatctaaaccactgctgcagttcctgtatgaacacgcgacccgttttgacttcacttgtcgtgtacgttggaagaaagaccaggtactggtttgggacaacctgtgcaccatgcatcgcgcggttccggactacgctggtaaatttcgctatctgacccgcactactgtaggtggtgttcgccctgcccgttaa

SEQ ID NO: 8 - DNA, 888 bp, synthetic construct
  source 1..888: mol_type = other DNA
  CDS 1..888: protein_id = 6; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgccttgtccccgttgtcgcaaaggttcgaaagaattgcagttcaaccactcacgggcgtgctgggagcagagatcacgggcgtggacttgcgcgagccccttgacgatagtacctggaatgaaattctggacgcgttccatacttaccaagtgatctactttcctggccaggctatcacgaacgaacagcacatagcattctcccggaggtttggccccgtcgaccctgtgcctcatctgaagtctatagagggatatccggaggtccagatgatccggcgtgaggcgaacgagtccgggagggttatcggagacgactggcacaccgatagcacattcctggacgcgccgcctgcggctgtggtgatgagagcgatcgacgttcctgagcacggcggcgacaccggcttcctttccatgtacacggcgtgggagacgctttcacctacaatgcaagcgactatcgagggacttaacgtggttcacagcgcaacccgcgtgttcggatcactgtaccaggcgcagaatcgcaggttttcaaacacctctctcaaagtgatggacgttgacgcgggtgacagggaaaccgttcatcctctggtggtgactcatccaggctctggccggaaggggctgtacgttaatcaggtctactgtcagcgcatagaaggtatgactgacgcagaatccaagccgctgcttcaattcctctacgaacacgccacgcgcttcgacttcacttgcagggtgcgttggaagaaggaccaagttctggtttgggataacctgtgcacgatgcatcgcgccgtgccagactacgcaggcaagttcaggtacctgacccggacgacggttggaggtgtccgcccagccaggtga

SEQ ID NO: 9 - DNA, 888 bp, synthetic construct
  source 1..888: mol_type = other DNA
  CDS 1..888: protein_id = 6; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgccgcactatctcccctcagtcagaggtttgaacgaatcgcagtacaaccattgaccggagttctaggagccgagataaccggggttgacctgagagaacccttggacgatagcacctggaatgaaatcctggacgcctttcatacctatcaggtaatctattttccaggacaagcgataacgaatgagcagcatatagcattctcgcggagattcggacccgtcgatcccgtacctcatctcaaatctatagaaggctacccagaagttcagatgattagacgtgaagcgaatgagagcggccgagttataggggatgattggcatacggactctacattcctcgacgcaccgccagccgctgtagtgatgcgtgcaattgatgtcccagaacacggtggcgatactggtttcttgtcgatgtacactgcatgggaaactctctcgccgacgatgcaggcaacgatagaaggactcaacgtggtacactcggccacccgggtctttggttccctgtatcaagcccaaaataggagattctccaacacgtccctgaaagtgatggacgttgacgcaggggatagagagactgtgcacccgcttgtggttacacacccgggctctggcaggaaaggcctttacgtcaaccaggtatactgtcagcgtattgaagggatgactgatgcagaaagcaaacctctcctccagtttctttacgaacatgcgacacggtttgactttacttgtcgcgtacgttggaaaaaggaccaagtcctcgtttgggataatctttgtaccatgcaccgagctgtgccggactacgctggcaaatttcgctatttaacgcggacgactgtcggaggcgtaagaccggcacggtga

SEQ ID NO: 10 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSMKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 11 - DNA, 888 bp, synthetic construct
  source 1..888: mol_type = other DNA
  CDS 1..888: protein_id = 10; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSMKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcgctgtctccactgtctcaacgtttcgaacgtatcgctgttcagccgctgaccggtgttctgggcgcagaaatcactggtgttgacctgcgcgagccgctggatgactctacctggaacgagatcctggacgcttttcacacctaccaggtcatttactttccgggtcaggctattactaacgagcagcacatcgcatttagccgtcgttttggtccagtggatcctgtgcctcatctgaaaagcatcgaaggttatccggaagttcagatgatccgtcgtgaagcaaacgaatccggtcgcgtcattggtgacgactggcacaccgactctaccttcctggatgctccgccggcagctgttgttatgcgtgccatcgacgtgccggaacacggtggtgataccggtttcctgtccatgtacactgcctgggagacgctgtctccaactatgcaagccaccatcgaaggcctgaacgttgttcatagcgcaacccgtgtattcggctctctgtaccaggcgcagaaccgtcgtttctctaacacctctatgaaagtaatggacgttgacgctggtgatcgcgaaactgttcacccgctggttgtgacccacccgggttctggtcgtaaaggtctgtatgtgaaccaggtttactgtcagcgcatcgagggtatgaccgacgccgaaagcaaaccgctgctgcagttcctgtacgaacacgctacccgctttgatttcacctgccgcgtacgttggaagaaagatcaggttctggtgtgggacaacctgtgcaccatgcaccgtgcggttcctgattacgcgggcaagttccgctatctgacccgtactaccgtgggtggtgttcgtccggcacgctaa

SEQ ID NO: 12 - DNA, 888 bp, synthetic construct
  source 1..888: mol_type = other DNA
  CDS 1..888: protein_id = 10; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSMKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgtcaccactttctcagaggttcgagaggatagcagtccagccgttgaccggtgttttgggtgcagagataacaggcgtcgaccttcgcgaacccctcgatgacagtacgtggaacgagatcctcgacgcgttccacacctatcaggtgatctactttccgggccaagcgatcacgaacgagcagcatattgcgttctcccgccggtttggcccagttgacccggttcctcatcttaagtctatcgagggatacccagaggttcagatgatccgccgtgaggcaaacgagagtggacgggtgatcggcgatgactggcacactgacagcactttcttggatgcacctcccgcagctgttgttatgagagctattgatgtgcctgagcatggtggcgacaccggttttcttagcatgtatacagcttgggagacattgtcgcccacaatgcaggcgacgatcgaaggcctgaacgtggtccatagcgctaccagggtctttggctccctgtaccaggcgcaaaatagacggttttcaaacacttctatgaaggtcatggatgtcgatgccggagacagagaaaccgtccatccactcgtcgtcactcatccaggaagcggccgcaagggtctctatgtgaaccaagtctattgccaacgcatcgagggcatgacggacgccgaaagcaagccgttgctccagttcctttacgagcacgctacaagattcgactttacgtgcagagttaggtggaagaaggaccaggtgctcgtgtgggacaacctgtgtacgatgcatcgggcagttccggactacgccggaaagttccgttacttgactaggaccacagttggtggggtcaggcctgcgagatga

SEQ ID NO: 13 - DNA, 888 bp, synthetic construct
  source 1..888: mol_type = other DNA
  CDS 1..888: protein_id = 10; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSMKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcgttatctcccctgtctcagagattcgagcgaattgccgtccagccgctcactggtgttctaggcgcagagataactggcgttgatctccgcgaaccactagatgactcgacctggaacgagatcctggatgcatttcatacgtatcaggtcatctattttccgggccaagcaattacaaacgagcaacacatagctttctctcgaaggtttggaccagtggatcccgtaccccatttgaagagtatcgagggctacccggaagtccagatgataagacgagaagcaaatgaatctggccgtgtgataggagatgactggcacacggactccacttttttagacgccccaccagcagctgtagtaatgcgcgctatagatgtacctgaacatgggggcgatactggatttctgtccatgtatactgcatgggaaacactatctcctactatgcaagccactatagagggtcttaatgttgtccattctgccactagagtttttggttcactctatcaggcgcaaaatcgtagattttctaacacctcaatgaaggtgatggacgtcgacgccggcgaccgagaaacagttcatcctctagtcgtaacccaccccgggtcggggcgtaagggcctgtacgtcaatcaggtttactgtcaaagaatagaaggcatgacagatgctgagagcaaaccattgctacagtttctgtatgaacatgcgacacggtttgattttacttgcagagttcggtggaagaaagaccaggtcctcgtctgggacaatctatgcacaatgcatcgcgcagtacctgactatgcggggaaattcagatacctgactagaacaaccgtagggggcgtacgaccagcaaggtga

SEQ ID NO: 14 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRFSNTSIKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 15 - DNA, 888 bp, synthetic construct
  source 1..888: mol_type = other DNA
  CDS 1..888: protein_id = 14; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSIKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcactgtctcctctgtctcagcgtttcgaacgtattgctgttcagccgctgactggcgttctgggcgctgaaatcactggcgttgatctgcgcgaaccgctggatgactccacctggaacgaaatcctggatgcgttccacacctatcaggtcatttacttccctggtcaggcaatcactaacgaacagcatatcgctttcagccgtcgtttcggtccggttgatccggtaccgcacctgaaatctatcgaaggctatccggaagtacagatgatccgccgtgaagcgaacgagtctggtcgtgttatcggtgatgactggcacactgactccaccttcctggatgcaccgccagcggcagtcgttatgcgtgcaatcgatgtcccagaacacggcggtgataccggcttcctgagcatgtacaccgcgtgggaaactctgtccccaactatgcaggctactatcgaaggcctgaacgtggtgcatagcgcgactcgcgtattcggctctctgtaccaggcacagaaccgtcgtttcagcaacacctccatcaaagtgatggacgttgacgctggtgaccgcgaaactgtccatccgctggtagttacccacccgggttctggtcgtaagggtctgtacgtaaaccaggtttattgccaacgcatcgagggcatgaccgacgccgaatccaaaccgctgctgcaattcctgtatgaacacgccacccgtttcgacttcacttgccgtgtccgttggaaaaaagaccaggttctggtttgggacaacctgtgcaccatgcaccgtgcggttccggattacgctggcaaattccgttacctgacgcgtaccaccgttggcggtgttcgtccggcacgttaa

SEQ ID NO: 16 - DNA, 888 bp, synthetic construct
  source 1..888: mol_type = other DNA
  CDS 1..888: protein_id = 14; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSIKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgccctgtcccctctgtctcagagatttgaacggatagccgtccagccgttgacaggcgtcctgggtgcggagatcacgggcgtcgacttgcgcgagcctctcgacgattcaacctggaacgagatcctcgacgcgttccatacataccaggtgatctatttccccggccaggcaatcaccaatgaacagcacattgccttctcccgccggttcggtccagtcgaccccgtgccacacctgaagtcgattgagggctaccctgaggtgcaaatgatcaggagggaggcgaacgaatctggccgtgtgataggtgatgattggcacaccgattccacattcctggatgcccctcctgcagccgttgttatgagggcaatagacgtcccggaacacggcggggacaccggattcctctccatgtacacggcctgggaaaccttgtccccgaccatgcaagctacaatcgagggactgaacgtcgtgcactcggcgacaagagttttcggctcactctaccaggcccaaaacagaaggttcagcaacacgtctatcaaggttatggacgtggatgcaggagatcgcgaaacggttcacccacttgtcgttacacacccgggctccgggcgcaaaggtctctacgttaatcaagtctattgccagaggatcgagggcatgaccgacgcagagtcgaaaccgctgctgcagttcctgtacgagcatgcaaccaggttcgactttacctgccgcgttcgctggaaaaaggatcaggtcttggtgtgggacaatctttgtaccatgcatagggccgtcccggactacgccggcaagtttagatacctgacgaggaccacggtcggcggagtcagaccggccagatga

SEQ ID NO: 17 - DNA, 888 bp, synthetic construct
  source 1..888: mol_type = other DNA
  CDS 1..888: protein_id = 14; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSIKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcactctcgcccctttctcaacgttttgaaagaattgctgtgcagccgctaacaggggttttgggagcggaaattaccggggtggatctacgtgaacctcttgacgacagcacgtggaatgagattctcgatgcgtttcacacataccaagtaatctattttcccggccaggcaataacaaatgaacagcatatcgcattctcccggagatttggccccgtcgatcccgtgccgcacttgaaatctatcgagggttacccggaagtccagatgattcggagagaggcgaatgaatccggacgggtcattggggacgactggcataccgatagtaccttcttggacgctccacctgcagctgtagttatgagggcaattgacgttcctgagcacggaggagataccggttttttgtctatgtatacagcatgggagactctttcccctactatgcaagcaacgatagaagggcttaatgtggttcatagcgcgactagggtgttcggatcgctgtatcaagcgcaaaacagaagatttagcaatactagcatcaaagtgatggacgtagatgccggcgatagagaaaccgtgcatcccttggtagtcactcaccctggtagtggaaggaaaggcctctacgtcaaccaggtatattgccaaagaatcgaggggatgacggatgcggagagtaaaccactattacaattcttgtacgaacatgccactcgatttgatttcacgtgtcgagtacgctggaaaaaagaccaggtactggtatgggataacttgtgcacgatgcatagggccgttccagactatgccgggaagttccgttatctaactagaacaactgtgggaggagtcagacctgcccgctga

SEQ ID NO: 18 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGAVIGDDWHTDSTFLDAPPAAVVMRAIDVPEHGGDTGFLSMYTAWETLSPTMQATIEGLNVVHSATRVFGSLYQAQNRRFSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 19 - DNA, 888 bp, synthetic construct
  source 1..888: mol_type = other DNA
  CDS 1..888: protein_id = 18; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GAVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcgctgtctcctctgtctcagcgttttgaacgtatcgccgttcagccgctgaccggtgttctgggtgccgaaattaccggcgtcgatctgcgcgaaccgctggatgacagcacctggaatgaaatcctggacgcgtttcacacctaccaggttatttactttccaggtcaggcgatcacgaacgaacagcacatcgctttcagccgtcgctttggtccggttgacccggttccgcacctgaaatctatcgaaggttatccggaagtgcagatgatccgtcgcgaagcaaacgagagcggcgcggtaattggtgatgattggcataccgattccaccttcctggatgctccgcctgcggctgtagttatgcgtgctattgatgttcctgaacacggtggtgacaccggtttcctgtctatgtacaccgcctgggaaacgctgtcccctacgatgcaagcgaccatcgagggtctgaacgtggttcactctgctacccgcgtttttggctccctgtatcaggcacagaaccgccgcttctccaacacctctctgaaagttatggatgtggatgccggtgatcgtgaaaccgtccacccgctggttgttacccatccgggtagcggtcgtaaaggtctgtacgttaaccaagtttactgccagcgtatcgaaggcatgaccgatgcggaatccaaaccactgctgcagttcctgtacgaacacgcaactcgttttgatttcacttgtcgtgttcgttggaaaaaagaccaagtgctggtgtgggacaacctgtgcaccatgcatcgtgctgtgccggattacgctggtaaatttcgttatctgactcgtactaccgttggtggcgtgcgtccggctcgttaa

SEQ ID NO: 20 - DNA, 888 bp, synthetic construct
  source 1..888: mol_type = other DNA
  CDS 1..888: protein_id = 18; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GAVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcactctccccactgtcccaacgcttcgaaagaatagccgtccaaccactgacaggcgtgcttggagccgagataactggcgttgacctccgtgaaccgctggacgactctacctggaacgagatcctggacgcgttccacacctaccaagtcatctacttccccggccaggcaattactaatgagcagcatatcgcgttctcaagaaggttcggacccgtcgacccagtcccacatctgaagtccatcgaggggtacccagaggtgcagatgattcgcagggaagcaaatgagagtggcgccgttataggtgatgattggcacaccgattccacgtttcttgatgcaccgccggctgcggttgttatgagagcaattgatgtgcctgaacacggtggggatacgggcttcctttccatgtacacggcctgggagaccctctccccgactatgcaggccaccattgagggcctgaacgtggtccacagcgcaaccagagtgttcggcagcttgtaccaagcccagaataggaggttctccaacacgtcccttaaggtgatggatgtggatgctggggaccgtgagaccgtgcacccacttgtggtcacgcatccaggctcagggcggaaagggctctacgttaaccaagtctattgtcagcgtatcgaggggatgacggatgcagagagcaaacctcttctgcagtttctctacgagcacgctactaggttcgattttacctgtcgcgtccgctggaagaaggaccaagttcttgtttgggacaacttgtgtaccatgcatagagctgtgccagactacgcgggcaagttcagatacctgacccgtactactgtcggcggcgttagacccgcaaggtga

SEQ ID NO: 21 - DNA, 888 bp, synthetic construct
  source 1..888: mol_type = other DNA
  CDS 1..888: protein_id = 18; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GAVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcggccttgtctcctctctcgcagagatttgaaagaattgcagtacagccgttaaccggggttttgggagctgaaataacgggtgtggatttacgtgagccgcttgacgacagcacctggaacgagattcttgatgcctttcatacgtatcaggtgatttatttccccgggcaggcaatcacgaacgagcagcacatcgcattttcccgccgatttggtcccgtcgacccagttccgcatctaaagagcatcgagggatatccggaagtccaaatgatacgacgagaagctaatgaatcgggcgcggtcataggtgatgactggcacactgattccacatttctagatgcccccccggcagccgtcgtaatgcgcgcgattgacgtgcccgagcacggaggcgacacaggtttcctctcgatgtacaccgcatgggaaacactgtcgccgactatgcaagcaacgattgaggggttgaacgtggtgcattccgcaacaagagtgttcggatcactataccaggcacagaatagacgcttttcgaatacctcgctaaaagtaatggacgtagatgctggagaccgtgagacagtacatcccctggtcgttacacatccgggtagcggccgcaaggggttgtacgtaaatcaagtttattgccaacggatcgaagggatgaccgatgctgaatcgaaaccactcctgcaatttttatatgaacatgcaacaaggttcgactttacttgtcgggttcgttggaagaaggatcaagtcttggtgtgggacaacttatgtacaatgcatcgagccgtacccgattatgcaggaaaatttcgatatctaacgcgcacgactgtagggggtgtgcgcccggctcgctga

SEQ ID NO: 22 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGDVIGDDWHTDSTFLDAPPAAVVMRAIDVPEHGGDTGFLSMYTAWETLSPTMQATIEGLNVVHSATRVFGSLYQAQNRRFSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 23 - DNA, 888 bp, synthetic construct
  source 1..888: mol_type = other DNA
  CDS 1..888: protein_id = 22; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GDVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctctgtctccgctgtctcagcgttttgaacgtatcgctgtacaaccgctgacgggcgtactgggtgctgagatcaccggcgtcgatctgcgtgagccgctggatgactctacttggaacgagatcctggacgccttccacacttaccaggtaatctatttccctggtcaggctatcacgaatgaacagcacatcgctttttcccgtcgtttcggtccggttgatccggttccgcacctgaaaagcattgaaggctacccggaagttcagatgatccgccgcgaggcaaacgaatctggcgacgtgatcggtgatgattggcacaccgattctacgttcctggacgctccgccggctgctgtagttatgcgcgctattgatgtaccggaacacggtggtgacaccggtttcctgtctatgtacactgcttgggaaaccctgtctccgaccatgcaggcaaccattgaaggcctgaacgtagtgcatagcgcaactcgtgtcttcggctctctgtatcaggcgcagaaccgtcgtttctccaacacttccctgaaagttatggacgttgacgcgggtgatcgtgaaaccgtgcacccgctggttgttacccacccgggttctggtcgtaaaggtctgtacgtcaaccaggtgtattgtcagcgtattgaaggtatgacggacgcggagagcaaaccgctgctgcagttcctgtatgaacacgctacgcgttttgacttcacttgccgtgttcgctggaaaaaagatcaggttctggtatgggataacctgtgcaccatgcatcgtgcggttccggactacgcaggtaaattccgttatctgactcgcactactgttggcggtgtacgcccggcacgttaa

SEQ ID NO: 24 - DNA, 888 bp, synthetic construct
  source 1..888: mol_type = other DNA
  CDS 1..888: protein_id = 22; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GDVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcactgagtcccctgtcacaaagatttgagaggattgctgttcaacccctcacaggggttctcggagcggagatcacaggcgtcgatttgcgggagcccctcgacgattctacctggaacgagatcctcgacgccttccatacgtaccaggtcatttatttcccggggcaagctatcactaacgagcagcatatcgcattttcgcgccgcttcggccctgtggaccctgtgccacacttgaagtccatagaaggttacccagaggtccaaatgattcggagggaggcgaatgagagcggcgacgtcattggtgatgactggcacactgactcaaccttccttgacgctccgcctgcagcggttgtgatgcgcgcgatcgatgtcccggagcacggaggcgacacgggcttcctctctatgtacacagcctgggaaacactgagccctaccatgcaggcgacaattgaagggttgaacgttgtgcattccgcaacacgggtgttcggatctctgtaccaggcccagaatcggaggtttagcaacacaagtttgaaggtgatggacgtcgacgccggggaccgcgagacagtccatcctctcgtggtgacccacccggggtccggcaggaagggactctacgtcaaccaggtgtattgccagcgtattgagggcatgaccgacgctgagagcaagcctctgctccagtttttgtatgagcatgcgacgaggttcgatttcacttgcagggtgaggtggaaaaaggaccaggtgctggtctgggacaatctctgtaccatgcacagggctgtccccgactacgcgggcaagttcaggtacctcacaaggaccaccgttggtggggtccggccggccaggtga

SEQ ID NO: 25 - DNA, 888 bp, synthetic construct
  source 1..888: mol_type = other DNA
  CDS 1..888: protein_id = 22; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GDVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcggcactaagccctctatctcagcggtttgaaagaattgccgtccagccactgactggagtcctgggagccgaaatcactggtgttgacttgcgagaacccttagacgattcaacctggaatgaaattttggacgcttttcatacctatcaagtgatttattttccggggcaggctatcacgaatgaacagcatatcgctttttctcgcagatttgggccggtagaccctgtgccacatttaaaaagtatcgagggataccctgaagtacaaatgattcgacgcgaagccaatgagtctggcgacgttattggggatgattggcacaccgactcaaccttcctcgatgcgcctcctgcggcagttgttatgagagcaattgatgtgccggagcacggaggggatacgggattcctgagtatgtatacagcttgggaaacgctgtcgccaacgatgcaagctaccatcgaagggctgaatgtcgtccattccgccaccagagtctttgggtctctttaccaagcacaaaatcggcgcttctctaacacctctttgaaggttatggacgtcgatgcgggagacagagagaccgttcatccacttgttgtaactcacccgggatccggaagaaaaggattgtacgtaaaccaggtctattgccaaaggatcgaaggaatgacagatgccgaaagcaagcctttgctgcaatttttatatgagcacgctacacggttcgattttacctgtcgtgtccgctggaaaaaagaccaagtcttggtctgggacaacctttgcaccatgcatagagcagtaccagattatgctggtaaattccggtatctaactagaacaactgttggaggggtgcgccctgcaaggtga

SEQ ID NO: 26 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGLVIGDDWHTDSTFLDAPPAAVVMRAIDVPEHGGDTGFLSMYTAWETLSPTMQATIEGLNVVHSATRVFGSLYQAQNRRFSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 27 - DNA, 888 bp, synthetic construct
  source 1..888: mol_type = other DNA
  CDS 1..888: protein_id = 26; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GLVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcggcactgtctccgctgtcccaacgttttgaacgtattgctgtccagccgctgactggtgttctgggcgctgagatcactggtgttgacctgcgtgaaccgctggacgattctacttggaacgagatcctggatgccttccacacctatcaggtcatttactttccgggccaggcgatcacgaacgaacagcacattgcgttcagccgtcgctttggcccggttgatccggttccgcacctgaaaagcatcgaaggctatccggaagttcagatgatccgtcgcgaggcgaacgaatctggtctggttattggtgacgactggcacaccgactccactttcctggatgctcctccggcagccgttgttatgcgtgcaatcgatgttccggaacatggcggtgataccggcttcctgtccatgtacaccgcttgggaaactctgtccccgaccatgcaggctaccattgaaggtctgaacgtcgtccactccgctacccgtgttttcggttctctgtatcaggcacagaaccgtcgtttctccaacacctctctgaaagttatggacgttgacgccggtgaccgtgaaaccgtgcatccgctggttgttacccacccgggttctggccgtaaaggtctgtacgtaaatcaggtctattgtcagcgcatcgaaggtatgaccgacgctgagtccaaaccgctgctgcaattcctgtacgaacacgccactcgttttgacttcacctgtcgtgtccgctggaaaaaagaccaggtactggtttgggataacctgtgcaccatgcaccgtgcggttccggactatgcgggtaaattccgttacctgactcgtactactgtgggtggcgtacgtccggctcgttaa

SEQ ID NO: 28 - DNA, 888 bp, synthetic construct
  source 1..888: mol_type = other DNA
  CDS 1..888: protein_id = 26; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GLVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcgcttagtccgctctctcaaagattcgagaggatagcggtccagccattgaccggcgtccttggtgcagaaataacaggcgtcgatctgagggagccactggacgattccacatggaatgagatcctcgacgcttttcatacgtaccaggtcatctacttcccgggacaggcaatcaccaacgagcagcacattgccttctcccgcaggttcggtcctgttgaccctgtgccccatctcaagagtatcgagggctaccctgaggtgcagatgatccggcgcgaagctaacgagagcggtttggttatcggcgacgactggcacaccgactcgacttttctggatgcgcctcctgccgcagttgtgatgagagctattgatgttcccgaacacggaggcgataccgggtttttgtcgatgtatactgcgtgggagacgctgtccccaaccatgcaggccaccatcgagggcttgaacgtggtccatagcgctacgagggtcttcggcagtctctaccaggcacagaacaggcggttcagtaacacctcattgaaggtgatggacgtggacgctggcgatcgcgagactgtgcatcccctggtcgttactcaccctggcagtggtagaaagggactctacgtgaatcaggtgtattgccaacggatagagggcatgacggacgccgagtcaaaaccccttcttcagtttttgtacgagcacgctacgcgcttcgatttcacatgtagagtgagatggaagaaggaccaagtgctcgtttgggacaatctttgtacgatgcacagggctgtgcctgattacgcggggaagttccggtacctgaccaggacaacagttggcggcgttagaccggcccggtga

SEQ ID NO: 29 - DNA, 888 bp, synthetic construct
  source 1..888: mol_type = other DNA
  CDS 1..888: protein_id = 26; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GLVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagctctatctcccttaagccagcgctttgaacgcatcgctgtacaacctctaactggagtccttggcgcggagataaccggcgtagatctgagagaaccgttggacgactccacctggaatgaaatcctagacgccttccatacataccaagtgatctattttcctggccaagccatcacaaatgaacaacatattgcgtttagccgcaggttcggaccggtagatcccgttccgcacttaaaaagcatagaaggttatcccgaggtccaaatgatacgaagagaagccaacgaatcgggactcgtaattggggacgactggcataccgattccactttcctagatgctccccccgcagccgttgtgatgagagctatagatgtgcctgaacatggaggtgatacagggtttcttagcatgtatacagcctgggagactctgtcgccgactatgcaggccaccatcgaggggttgaatgttgttcactctgccactagagtatttgggagcctataccaagctcagaatcgccgattctctaatacgtcgcttaaggtcatggacgtagacgcgggagatcgggaaaccgtgcaccccctggttgtaacgcatcctggctccgggcgcaaaggcctttacgttaaccaagtctactgtcagcgaattgagggaatgacagatgccgaatctaagccgctgttacaatttttgtatgaacatgcgactcgctttgatttcacgtgtcgcgtaaggtggaaaaaggaccaagtactagtctgggataatctgtgtactatgcatcgggccgtacccgattatgcgggaaagtttcgctatttgacacgtacgacagtcgggggtgttaggccagcccgctga

SEQ ID NO: 30 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGRVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 31 - DNA, 888 bp, synthetic construct
  source 1..888: mol_type = other DNA
  CDS 1..888: protein_id = 30; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgtctccactgtctcagcgtttcgaacgtattgcggtacagcctctgaccggtgtactgggtgccgaaatcaccggcgtcgatctgcgcgaaccgctggatgatagcacctggaacgagatcctggacgctttccacacttatcaggtaatctatttcccaggtcaggcgattaccaacgaacagcacatcgccttttcccgccgtttcggtccggtagatccagtgccgcatctgaaaagcattgagggctacccggaagttcagatgattcgtcgcgaagcaaacgaatccggtcgtgttatcggtgacgattggcacaccgattctactttcctggacgcacctccggctgctgttgttatgcgtgctatcgacgtaccagaacacggcggcgacactggtttcctgtccatgtacaccgcatgggaaaccctgagcccgaccatgcaagcgaccatcgaaggcctgaacgttgttcactctgctacgcgtgtgtttggttctctgtaccaggcacagaatcgtcgctggtctaacacttccctgaaagttatggacgtggatgcgggtgatcgtgaaaccgtgcatccgctggttgttactcatccaggtagcggccgtaaaggcctgtatgttaaccaggtttactgccagcgtatcgaaggcatgaccgacgcggagtccaaaccgctgctgcagttcctgtatgaacacgcaacccgttttgattttacctgccgcgttcgttggaaaaaagaccaggttctggtatgggataacctgtgcactatgcaccgtgctgttccggattatgctggtaagttccgctacctgactcgtaccactgttggtggtgttcgcccggctcgctaa

SEQ ID NO: 32 - DNA, 888 bp, synthetic construct
  source 1..888: mol_type = other DNA
  CDS 1..888: protein_id = 30; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagccctctccccacttagccaaaggttcgagaggattgctgttcaaccgctgaccggagtgctgggggcggagatcactggtgtcgacctccgggaaccactcgacgacagtacgtggaacgaaatccttgacgcgtttcacacttaccaagtgatctatttccctggacaggcgatcacaaacgagcaacacatcgccttctcccgccgctttggaccagtggacccagttccgcaccttaaatccatcgaagggtaccccgaggtccagatgatccggcgcgaggcgaacgagtcggggcgggttataggcgatgactggcacactgatagtacattcctggacgccccccccgccgcggtggttatgcgcgcgatcgacgttccggagcacggtggcgacaccggattcttgagcatgtacactgcctgggagactctgagcccgacgatgcaagctacgatcgagggattgaatgtggtccactcagcgacgcgcgtgttcggatcactctaccaagcacaaaacagaagatggtctaacactagcctcaaggtgatggacgtggatgcaggcgatagggagacggtccacccactcgtggttacacatccgggctcaggccgtaagggactttacgtcaaccaggtgtattgccagcgcatcgagggaatgaccgatgcagagagcaaaccgctgctgcaattcctttacgagcacgccacccggttcgatttcacatgccgcgttcgttggaagaaggaccaggtgctcgtctgggacaacctttgcactatgcaccgtgctgttccagactacgctggtaagttcaggtacctcaccaggactacggtcggcggcgtccgcccggcgaggtga

SEQ ID NO: 33 - DNA, 888 bp, synthetic construct
  source 1..888: mol_type = other DNA
  CDS 1..888: protein_id = 30; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgccgccctaagcccattatctcagagatttgaaagaattgctgttcaacctctaaccggcgtgttaggcgctgaaataaccggagttgacttgcgtgagccccttgatgactcgacctggaatgaaattctagacgccttccacacatatcaggtaatttattttccaggtcaggcaattacgaatgaacagcacatagccttcagtaggaggttcggtcccgtagaccccgtgccacacctaaaaagcattgaaggttatcccgaagtccagatgattcgaagagaggcaaatgaatcgggtagggtcatcggagacgactggcatacggactcaacttttcttgatgcgccaccggcagctgtagtgatgagagcaattgatgtgcccgagcacggtggagatactgggtttctaagtatgtacaccgcttgggagacgctttcgccgactatgcaggcgaccatcgaaggattaaatgtcgttcactcggctacgcgggtattcgggagtctataccaagctcagaatcgtagatggtccaatacatctttaaaagtaatggacgtcgatgccggcgatcgtgaaactgtacatccgttggttgtaacccatcctggaagcgggcgtaaaggactatacgtcaaccaagtgtactgccaacgcattgaaggtatgacggacgctgaatcgaagccgttgctccaattcttgtacgagcatgcaacccgattcgactttacctgtcgggtacgctggaagaaagatcaggtcctggtgtgggataatctatgtactatgcatagagcagttcctgactacgcgggcaaatttagatacttgacccgtacgacagtcgggggagtgagacctgcgagatga

SEQ ID NO: 34 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNRRFSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 35 - DNA, 888 bp, synthetic construct
  source 1..888: mol_type = other DNA
  CDS 1..888: protein_id = 34; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactgagcccgctgtctcaacgttttgaacgtattgcagttcagcctctgacgggcgttctgggtgcagaaattactggcgtggatctgcgtgaaccactggacgactctacctggaatgaaatcctggatgcgttccacacttaccaggttatttactttccgggccaggctatcaccaacgagcagcacattgctttctcccgtcgtttcggtccggtggacccggttcctcatctgaaatccattgaaggctatccggaagtacagatgatccgtcgtgaagccaacgagtctctgcgcgtaattggcgacgactggcacaccgactccacttttctggacgctccgccggcagctgtagttatgcgtgctattgatgttccggagcacggtggtgacaccggtttcctgtccatgtacaccgcttgggaaaccctgtccccgaccatgcaggcaaccatcgagggcctgaacgtagtccactccgcaacccgtgtattcggctctctgtaccaggctcagaatcgccgtttcagcaacacctccctgaaagttatggatgttgatgcgggcgatcgtgaaaccgttcacccgctggttgtgacccacccgggttctggtcgcaaaggtctgtacgttaaccaggtttattgccagcgcattgaaggtatgaccgatgcagaatccaaaccactgctgcagttcctgtatgaacacgcgacccgtttcgatttcacctgccgcgtgcgttggaaaaaagatcaggtactggtgtgggacaacctgtgcactatgcatcgcgcagtgccggactatgcaggtaaattccgttacctgacccgtaccaccgttggtggtgttcgtccggcacgttaa

SEQ ID NO: 36 - DNA, 888 bp, synthetic construct
  source 1..888: mol_type = other DNA
  CDS 1..888: protein_id = 34; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctctcaccgctcagccaaaggtttgagagaattgctgttcagccgctcaccggtgtgttgggagccgaaattacgggagtcgatctcagagagcctcttgacgattccacctggaacgagatcttggatgcattccacacgtatcaggtcatttactttccaggacaggccatcacgaacgagcagcatattgcattcagccgtcggttcggaccagtggacccggttcctcatctgaagtctatagaaggttacccagaggtgcagatgattaggcgcgaggccaacgaaagcctcagagtcatcggcgatgactggcacactgactccaccttcctcgacgcccctcctgctgctgtcgtcatgagagctattgatgtccccgagcatggcggagatacgggcttccttagcatgtacacggcctgggaaaccctgtcccccactatgcaggctaccattgagggccttaacgtggtgcattctgccactcgggtgtttggctcgttgtaccaggctcagaacaggaggttctccaacacatctcttaaagttatggatgttgatgccggtgatagggagactgtgcatcctctcgtcgttacccacccaggctcaggacgtaaaggactgtacgtgaaccaggtgtactgtcaaaggatcgaaggcatgacggatgccgaaagcaagcccctcttgcaattcctctacgagcatgcgacgcggtttgatttcacctgcagggtgcggtggaagaaggaccaggtcctcgtgtgggataacctgtgcactatgcacagggccgttccggattatgcgggcaagtttagataccttacaagaaccacagtcggtggcgtcagacctgccaggtga

SEQ ID NO: 37 - DNA, 888 bp, synthetic construct
  source 1..888: mol_type = other DNA
  CDS 1..888: protein_id = 34; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctttcgccccttagtcagagattcgaaaggatagcggtccaacctctcaccggtgtcctgggagcggagataaccggcgtagacttgcgcgaacctcttgacgattccacatggaatgagattctcgatgcttttcatacgtaccaagtcatttatttccctggccaagccatcaccaacgaacaacacattgcattcagtcgccgttttgggccggtcgatccggtaccacatttgaaaagcatagaaggataccccgaggtgcagatgattcggagggaggcaaatgagtctctgcgtgtaataggcgacgattggcatacggattcaacctttctcgatgcaccaccggcagcagttgtaatgagagccattgatgtaccggagcacggaggagatacgggcttcttgtctatgtatacggcatgggagactttatcgccaactatgcaggcaactatagaggggttaaatgtcgttcactcggctactcgagtttttgggagcctttatcaggcccagaaccgacgcttctccaatacatcgttgaaggtcatggatgtggatgccggagatcgcgaaacggtacacccacttgtggtaacacaccctgggtcagggcgcaaaggcctttatgttaaccaagtctattgccagcggatagaaggaatgaccgacgcggagtctaaaccactgttgcagtttctctatgagcatgcgaccagattcgattttacgtgccgtgtaagatggaagaaagatcaagtcttagtatgggataacctgtgcacgatgcacagagccgttcccgattatgccgggaaattcagatacttgacgagaacaacagtaggaggtgttcgacctgcgcggtga

SEQ ID NO: 38 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 39 - DNA, 888 bp, synthetic construct
  source 1..888: mol_type = other DNA
  CDS 1..888: protein_id = 38; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcgctgtctcctctgtctcagcgtttcgaacgtatcgctgttcagccgctgaccggtgttctgggcgcagaaattaccggcgtagatctgcgtgagccgctggacgactctacctggaacgaaatcctggacgccttccacacttaccaggtgatctatttcccgggtcaggcaatcaccaacgaacaacacattgctttctctcgccgcttcggcccggtagatccggtacctcacctgaaatctattgagggttacccggaggtccagatgatccgccgtgaggcgaacgagtccggcggtgttatcggtgatgactggcatacggattctacctttctggacgcacctccggcagcagtggtgatgcgtgcaatcgacgtgccggaacatggcggcgatactggcttcctgtctatgtataccgcgtgggagaccctgtctccgaccatgcaggccaccatcgaaggcctgaacgtagttcactccgctactcgtgttttcggcagcctgtaccaggcgcaaaaccgtcgttggagcaacacctccctgaaagttatggacgtcgatgccggtgatcgtgaaaccgttcacccgctggttgtgacccacccgggctctggccgtaaaggtctgtacgtcaatcaggtgtattgtcaacgcattgaaggcatgaccgacgcagaatctaagccactgctgcagtttctgtacgaacacgctactcgcttcgacttcacctgtcgtgttcgttggaaaaaagatcaggttctggtttgggacaacctgtgtaccatgcaccgtgcagttccggactatgctggtaaattccgttatctgactcgcaccaccgtgggtggtgtacgtccggcgcgttaa

SEQ ID NO: 40 - DNA, 888 bp, synthetic construct
  source 1..888: mol_type = other DNA
  CDS 1..888: protein_id = 38; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcattgagcccgctttctcagcgtttcgagagaatcgcagtgcagcccctgacgggcgtgctcggcgctgaaatcacgggcgtggatctccgggaaccactggatgatagcacatggaacgagatccttgatgcgttccacacttaccaagtcatttatttccccggccaggctatcacaaacgagcagcatatcgctttctcgaggaggttcggcccggtggaccccgttccgcacctcaagtccattgaaggctatccagaggtccaaatgatccggagagaggctaacgaaagcggtggcgtcatcggggatgactggcataccgattccacgtttttggacgcccccccagcagcggtggtgatgcgcgcaatcgatgttccagagcatggcggtgatactggcttcctctcgatgtatacagcatgggagacactctcgccaaccatgcaggcgacgatagaggggctcaatgtcgttcacagcgccacccgcgtcttcgggagcttgtatcaggcccagaatagacggtggagcaacactagcctcaaggtgatggacgtcgacgctggcgatagagagaccgtgcatccgctggtcgtgactcacccgggatcaggtaggaagggcttgtatgttaaccaggtttactgtcagaggatcgagggcatgaccgacgccgaatctaagcccttgctgcagttcctctacgagcatgctaccagattcgacttcacttgcagggttaggtggaagaaggatcaggtccttgtctgggacaatctctgcacaatgcatagggctgtgccggactacgctgggaagttccggtatctcacgaggactacggttggcggcgtgcgcccagcgcggtga

SEQ ID NO: 41 - DNA, 888 bp, synthetic construct
  source 1..888: mol_type = other DNA
  CDS 1..888: protein_id = 38; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctttaagcccacttagccagaggtttgaacgtattgctgtccaaccactcactggcgttttgggagctgaaataacaggtgttgatctcagagaaccactcgatgattcaacctggaacgaaatcttggacgcatttcatacataccaagtgatttatttcccaggccaagctattaccaatgaacaacatatcgctttcagcagaagattcggtccagttgaccctgtacctcatttgaagagtatcgagggatacccagaagtgcaaatgattagacgtgaagctaacgagtccgggggagttatcggagatgattggcataccgattcaacttttctcgatgcaccgcctgcagctgtggtgatgagggctatagatgtcccagagcacggaggtgatactggctttttgtctatgtacactgcctgggaaaccttaagccctacaatgcaagctacaattgagggtttgaatgttgtgcatagtgccactagggtgttcggatctctttaccaggctcaaaatagaagatggtcaaatacaagtcttaaggtaatggacgttgatgctggtgatcgtgagactgtgcacccgcttgtagtgacccaccctggtagtggtaggaagggtttatatgtcaaccaagtatattgccagcgtattgaaggtatgactgatgcagagtcaaagccccttcttcaatttttgtacgaacacgcaactcgtttcgattttacttgcagagtgaggtggaagaaagatcaggttcttgtctgggacaacttatgtacaatgcatcgtgcagtgcccgactatgctggaaaatttagatacttgactcgaaccactgtaggaggcgtgcgtccggcacgatga

SEQ ID NO: 42 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 43 - DNA, 888 bp, synthetic construct
  source 1..888: mol_type = other DNA
  CDS 1..888: protein_id = 42; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcactgtctccgctgtctcagcgcttcgaacgtattgctgtgcagccgctgaccggcgttctgggtgctgaaatcactggcgtagacctgcgtgaaccgctggatgattccacctggaacgagattctggatgcgttccacacctatcaggttatctatttcccgggtcaggctatcactaacgaacaacacattgctttctcccgccgctttggtccggtcgacccggttccgcacctgaaatctatcgaaggctacccggaagtgcagatgattcgtcgcgaagccaacgaaagcctgcgtgttatcggcgatgattggcacaccgacagcaccttcctggacgctccgccagcagcggtggttatgcgtgccatcgacgttccggaacacggcggtgatactggtttcctgtctatgtataccgcgtgggaaactctgtctccgactatgcaggctaccattgaaggtctgaacgtggtgcactctgcgacccgtgtgttcggcagcctgtaccaggcgcagaaccgtcgctggagcaacacctctctgaaagttatggacgttgacgcaggcgatcgtgaaaccgtgcacccgctggttgtgacccacccgggttctggtcgcaaaggcctgtacgtgaaccaggtatactgccaacgtatcgaaggtatgactgatgcagaatctaaaccgctgctgcagtttctgtacgaacacgcgactcgtttcgactttacttgtcgtgtgcgttggaagaaggatcaggtgctggtttgggacaacctgtgtaccatgcaccgtgccgttccagattacgcgggtaaattccgttacctgacccgcaccaccgtcggtggcgtccgcccagctcgttaa

SEQ ID NO: 44 - DNA, 888 bp, synthetic construct
  source 1..888: mol_type = other DNA
  CDS 1..888: protein_id = 42; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcgctttcccccctgagccaacggtttgagaggatcgccgtccaaccactcacgggcgtgctgggcgcggagatcactggtgtagatctccgtgaacctcttgatgattcaacgtggaatgaaatacttgacgcgttccacacatatcaggtcatctacttcccggggcaagcaataacgaatgagcagcacatagcttttagtcgacgcttcggccccgtggatcctgtgccgcacctcaaatctattgaaggatacccggaggtacagatgatccggagagaggccaacgaatcgttgcgagtgattggtgacgactggcacaccgacagcacctttctggacgccccaccagcagcagtcgtgatgcgtgccattgatgtgccagagcacggcggcgacactggttttctctccatgtacacggcgtgggagacgctctcacctacaatgcaggccacaattgaggggctgaacgtggtgcattctgctactagagtttttggttcattgtaccaggcgcagaaccgccgctggtccaatacctcgctaaaggtcatggacgtcgatgctggagacagagaaactgttcatccgctggtagttacacatcctggatctgggaggaagggactatatgtcaaccaagtttactgccagaggatcgagggcatgactgatgccgagagcaaaccacttctgcagttcctctatgaacatgcaacgaggttcgacttcacctgccgggtgaggtggaagaaggatcaagttcttgtgtgggataatttgtgtaccatgcaccgcgctgtccccgactacgccggcaagttccgctatttaaccaggacaacagttggtggggttcggcctgctcgttga

SEQ ID NO: 45 - DNA, 888 bp, synthetic construct
  source 1..888: mol_type = other DNA
  CDS 1..888: protein_id = 42; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctttgagtcctcttagccaacgatttgaaaggatcgctgtccaacctttgactggtgtattgggtgctgagattaccggcgttgatcttcgtgagccactcgacgactctacatggaatgaaattctggatgcttttcatacatatcaggtgatttattttccagggcaagctattaccaatgagcagcatattgctttttctcgtaggttcggtcccgtggatccggtacctcatctgaaatcaattgaggggtacccggaggttcagatgatacgtagggaggctaatgagtcccttcgagtcattggtgatgattggcatacagatagtacatttctcgatgccccacctgctgctgtggttatgagggccattgatgttcccgagcatggtggagacacagggttcctttcaatgtatacagcctgggagactctctccccaacaatgcaggccactatcgagggtcttaatgtagtgcattcagctacaagggttttcgggagcctttaccaggcacaaaataggaggtggtcaaatacaagcttgaaagttatggacgtcgatgctggcgacagagaaacagtgcatccacttgtggtgacccatccaggatccggaagaaagggattgtacgtaaaccaggtgtactgtcaacgtattgagggaatgactgatgcagaaagtaaaccactcctccagttcttgtatgagcatgcaactaggttcgattttacctgtagggttagatggaagaaggatcaagttctcgtttgggataacctctgtaccatgcatcgtgcagttcctgattatgcagggaaatttcgttacttgactagaacaaccgtgggaggagttaggccagccagatga

SEQ ID NO: 46 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHSDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 47 - DNA, 888 bp, synthetic construct
  source 1..888: mol_type = other DNA
  CDS 1..888: protein_id = 46; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctctgtctccgctgtctcagcgttttgaacgtatcgcggttcagccgctgaccggtgttctgggtgctgaaatcaccggcgtagacctgcgtgaaccgctggacgattctacctggaacgaaatcctggatgctttccacacctaccaggtgatctactttccgggccaggctattactaacgagcaacacattgcgtttagccgtcgcttcggcccggtggacccagttccgcacctgaaatctatcgaaggctatccggaggttcagatgatccgtcgtgaggccaacgaaagcggtggtgtgatcggtgacgactggcactccgactccaccttcctggacgcgccgccggcggcagttgttatgcgtgctattgacgttccagaacatggtggtgatacgggtttcctgtccatgtataccgcatgggaaaccctgagcccaactatgcaagcaactatcgaaggcctgaacgttgtccacagcgccacccgcgtattcggtagcctgtatcaggcccagaaccgccgctggagcaatacctctctgaaagtgatggacgtggatgctggcgaccgtgaaaccgttcaccctctggttgtaacccatccgggttctggccgtaaaggtctgtacgttaaccaggtgtactgtcagcgtattgaaggtatgaccgacgcagaatccaaacctctgctgcagttcctgtatgaacacgcaacccgttttgatttcacctgtcgcgttcgttggaaaaaagaccaggttctggtatgggataacctgtgcaccatgcaccgcgctgttccggactacgcgggtaaattccgctacctgactcgcaccaccgttggtggcgttcgtccggcacgttaa

SEQ ID NO: 48 - DNA, 888 bp, synthetic construct
  source 1..888: mol_type = other DNA
  CDS 1..888: protein_id = 46; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcattgagtccattgtcgcagcggtttgagcggattgcggtccagcccctgacaggcgtgttgggagctgagattaccggcgtggacctccgcgagccgcttgacgactcaacctggaatgagatcctggatgcctttcatacctatcaagtgatttactttcccgggcaggctattacgaacgagcagcatatagcgttctccaggaggttcggcccagtggatccggtcccacatctgaagtccattgaaggatatccagaggtgcagatgatccggagggaggctaacgagagtggcggtgtgattggcgacgactggcactcggactcgactttcctcgacgccccgccagcggccgtcgtgatgagggctatcgacgtgccagaacacgggggagacacggggttcttgagcatgtacaccgcttgggaaaccctcagtcctaccatgcaggccacgatcgaaggcctgaacgttgtgcactcagccacccgggtgttcgggtcgctgtaccaggctcaaaatcggagatggtccaacacctccctgaaggtgatggacgttgatgccggggatcgcgagaccgtccatcccttggtggtgacccacccgggaagcgggaggaagggcctctacgttaaccaagtctattgccaacgcatcgaaggaatgaccgatgcagagtcgaagccgctcctgcagttcctctatgaacacgcaacgcgttttgacttcacgtgcagggtcaggtggaagaaggaccaagttctcgtgtgggacaacctctgcacaatgcacagagcagttcccgactacgctggcaagtttaggtatctcacccggaccacggttggcggggtgcgcccggcgcgttga

SEQ ID NO: 49 - DNA, 888 bp, synthetic construct
  source 1..888: mol_type = other DNA
  CDS 1..888: protein_id = 46; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcactgagtccgctgtctcagcgttttgagagaatcgcagtccaacccttgactggcgtcctcggagctgagatcactggcgtggatcttcgtgaacccctcgatgattcaacatggaatgagatcctcgatgcttttcatacctatcaggtgatttattttccaggacaagcaatcaccaatgaacaacatattgccttcagcagaagatttggtcctgtggatccagtgccccacttgaaaagcatcgaaggttacccggaagtccaaatgatcagacgagaagctaacgaatcagggggagttataggcgacgattggcatagcgactcaactttcttggacgcacctccagcagctgttgttatgcgtgccattgatgtgccagaacacggtggggataccggcttcctctcaatgtacactgcttgggagacactttctccaactatgcaggctactattgaaggtctcaacgtagtgcatagcgcaactcgagtttttggctccttgtaccaggctcaaaacagacgttggagcaatacatctcttaaagttatggacgttgacgctggtgatcgagaaaccgttcatcctttggttgttacacatccaggttccggtagaaaaggtctttatgtgaaccaagtgtactgtcaaagaatagaaggtatgacagatgccgagtccaaacccttattgcaattcttgtatgaacatgcaaccagattcgactttacatgcagagttaggtggaagaaggatcaagttctcgtatgggacaatttgtgcaccatgcaccgtgcagttccagattatgctggaaagttcagatatctgacaagaaccactgttggtggagtgcgtccagccaggtga

SEQ ID NO: 50 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPTPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 51 - DNA, 888 bp, synthetic construct
  source 1..888: mol_type = other DNA
  CDS 1..888: protein_id = 50; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactgtctccgctgtctcagcgttttgaacgtatcgctgtacaaccgctgactggcgtactgggtgctgaaatcactggcgtagacctgcgtgaaccgctggacgattctacctggaacgaaattctggatgcgttccacacctaccaggttatctacttcccgggtcaggcaattaccaacgagcaacatattgctttctctcgtcgttttggtccggtggacccgaccccacacctgaaaagcatcgagggctacccggaagttcagatgattcgtcgtgaagccaacgaatctggtggtgtcattggtgacgactggcacaccgacagcaccttcctggacgcacctccagcagcggttgtcatgcgtgccatcgatgtaccggaacacggtggtgataccggctttctgtctatgtataccgcttgggaaaccctgtctccgactatgcaggctaccatcgaaggtctgaacgttgttcacagcgctacccgcgttttcggttctctgtaccaagcacagaaccgtcgttggtctaacacttctctgaaagtgatggatgtagatgcaggtgatcgtgaaactgtgcacccactggttgttacgcacccgggtagcggtcgcaaaggtctgtacgtaaaccaggtttactgtcaacgtattgagggtatgaccgatgcagaaagcaaaccgctgctgcagttcctgtatgaacacgctacccgtttcgatttcacctgtcgcgtccgttggaagaaagatcaggttctggtgtgggacaacctgtgcaccatgcaccgtgctgttccggactacgctggcaaattccgttacctgacgcgtacgaccgttggtggtgttcgtccggcgcgttaa

SEQ ID NO: 52 - DNA, 888 bp, synthetic construct
  source 1..888: mol_type = other DNA
  CDS 1..888: protein_id = 50; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggctctctcccctctctcccaacgcttcgaaagaattgcggtgcagccacttactggtgttctcggggccgagatcaccggtgtcgatctgagagaaccgctcgacgactctacctggaacgagatcctggatgcgttccacacgtaccaggtcatctactttcccggccaagccatcaccaacgagcagcacattgccttcagccggcgttttggaccagtcgatcccactccacatctgaagtccattgaggggtatccagaggtgcagatgataaggagggaggcgaacgagtcaggaggggtgataggcgacgactggcacacagactcgaccttccttgacgctcctcctgccgccgtcgtcatgagagctatcgatgtgcctgagcacggtggcgacacgggttttcttagcatgtacacggcctgggagaccctctctcctacaatgcaggccactatcgaaggcttgaacgtggttcattccgcaacgagagtctttgggtcactctaccaggcgcagaaccgcaggtggagcaatacgagcttgaaggtgatggacgtcgacgccggtgatagagagaccgtgcacccgctcgtggttactcaccccggcagcggacgcaaagggttgtacgtgaaccaggtttactgccagcgtatagagggcatgacagacgccgagagcaagcctcttttgcagttcctctatgaacacgcaacgcgcttcgactttacttgccgcgtgcgttggaagaaggatcaggtgcttgtctgggacaacctttgcactatgcatagagcagttccagactacgccggtaaattccgctacttgactaggaccaccgtcggcggtgttaggcctgcaaggtga

SEQ ID NO: 53 - DNA, 888 bp, synthetic construct
  source 1..888: mol_type = other DNA
  CDS 1..888: protein_id = 50; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcactgtctcccttgtctcaacgtttcgagcggattgctgtacaacctttgacaggggtgttgggagcagaaatcaccggagtagacctaagggaacctcttgatgattcaacgtggaacgagattctagatgcctttcacacctaccaggtaatctattttccaggacaggctattactaacgagcaacatattgctttcagtcgtcgctttggacccgtggacccaacacctcacttaaaaagtattgagggatatccggaagttcagatgatccgacgcgaagccaatgagagtggcggtgtgatcggagatgactggcatacggacagtacgtttcttgatgcccctcctgcagctgtggttatgcgggctattgatgtacctgaacatggaggggacacgggattcctctcgatgtataccgcgtgggagacgttaagccctacgatgcaagctaccattgaaggcctgaacgtcgtgcattctgccacccgcgtgttcggcagtctctatcaagcgcagaatcggcgttggtccaatacgagcttgaaagttatggatgtagatgccggagaccgcgagacggtccatccactggtggttactcatcctggctcaggacgaaaaggactatacgtgaatcaggtatactgtcagagaatcgaagggatgacagatgccgaaagcaaacccttactccagtttttgtacgaacatgccacccgttttgatttcacatgtcgtgtacgttggaagaaagatcaagtcttagtctgggataatctctgcactatgcatcgcgcggtccccgactatgcgggcaaattccggtacttgacgcgtacgacggtcgggggcgtccgacctgcgagatga

SEQ ID NO: 54 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNWRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 55 - DNA, 888 bp, synthetic construct
  source 1..888: mol_type = other DNA
  CDS 1..888: protein_id = 54; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcgctgtctccactgtctcagcgcttcgaacgtattgctgttcagccgctgaccggtgttctgggtgcagagatcaccggcgttgatctgcgtgaaccgctggatgactctacctggaacgaaattctggacgcgttccacacctatcaagtaatctattttccgggtcaggccatcaccaacgaacagcacattgctttctcccgtcgtttcggtccagttgatccggttccacatctgaaatccatcgaaggttacccagaagtacagatgattcgtcgcgaagcaaacgagtctggcggtgtgatcggtgatgactggcacaccgattccactttcctggacgctccgccggcagcggttgttatgcgtgcaatcgacgttccggaacacggcggcgacactggtttcctgtccatgtacaccgcttgggagaccctgtctccgacgatgcaggcgactatcgaaggtctgaacgttgtccactccgctacccgcgttttcggctctctgtatcaggctcagaactggcgttggagcaacacctccctgaaagttatggacgtggatgctggcgaccgtgaaaccgtacatccgctggtggtgactcatccgggttctggtcgtaaaggcctgtacgtgaaccaggtttactgccagcgcatcgaaggtatgaccgacgcggagtctaaaccgctgctgcagttcctgtacgaacacgctacgcgtttcgacttcacctgccgtgtgcgttggaaaaaagaccaggttctggtctgggataacctgtgtaccatgcaccgcgcagttccggattacgcaggtaaattccgttacctgactcgtactactgtaggtggtgtccgcccggcgcgttaa

SEQ ID NO: 56 - DNA, 888 bp, synthetic construct
  source 1..888: mol_type = other DNA
  CDS 1..888: protein_id = 54; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactgtctcctttgtcgcagagattcgaaagaattgcagtccagcccctcactggagtcctcggagcggaaatcacaggcgttgacctccgcgagcctctggacgactccacctggaacgaaattctcgatgccttccacacataccaggttatctacttccctggccaagcgatcactaacgagcagcacattgcctttagccgccgctttggtcctgtggatcctgtcccccatcttaagagcatcgagggctacccagaagtccagatgatccggcgcgaggcgaatgagagcggcggcgtgatcggggacgactggcacactgactctaccttcttggacgctccgcccgcggctgtcgtcatgcgtgccatcgacgtccccgagcacggtggtgacaccggctttttgtcaatgtacaccgcctgggaaactctcagtcctacgatgcaggctaccatcgagggtctgaatgttgtccatagcgcgacccgggtgttcggctctctgtaccaggcccagaactggcggtggtctaacacctccctgaaagtcatggacgtggatgccggcgacagggaaaccgtccacccgctggtcgtgacccatcccggttcaggccgtaagggtttgtacgttaaccaggtttactgtcagcggatcgaggggatgacagacgctgagtccaagcccttgcttcaattcctgtatgaacacgccacgagattcgatttcacgtgcagggtgcgctggaagaaggatcaagtccttgtgtgggacaacttgtgcacaatgcacagggcggtcccggactatgctggtaagttcaggtacctgactcgtacaaccgtgggcggagtgcgtcctgccagatga

SEQ ID NO: 57 - DNA, 888 bp, synthetic construct
  source 1..888: mol_type = other DNA
  CDS 1..888: protein_id = 54; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctttgagccctctctcacaaagatttgaaagaatcgcggtccaaccgcttaccggcgtcttaggagccgaaataacaggagtagatttaagagaacctctagatgattctacctggaatgagattttagatgcgttccacacatatcaagttatctacttcccgggccaagcgattaccaatgagcagcacatcgcgttttctagacgtttcggaccagtggatcccgtgccgcatctaaaatccatcgagggatatccagaggtccagatgattagacgtgaggcgaacgagtccggaggggtaattggagacgactggcacaccgatagcacctttcttgacgccccccccgcagcggttgttatgcgtgcgatcgatgtcccagaacatggcggagacacggggtttctctccatgtacacggcgtgggagacgctgagcccgacgatgcaagcgacgatagaaggattgaacgtggttcactccgccacacgtgtgttcggttctttgtaccaagctcaaaattggcggtggtcgaacacgagcctgaaagtaatggacgtagatgcaggcgatcgggagacagttcaccccttagtggtcacccacccaggatcgggcaggaaggggctctatgtgaaccaggtatactgtcaaagaattgaaggtatgaccgatgccgaatccaaacctctcctgcagttcctgtatgagcatgcgacccgatttgatttcacctgccgtgtgcgttggaaaaaagatcaagtgctggtttgggacaatctttgcacaatgcatcgcgcagtgcccgattatgcgggaaagttcagatatttgacccgtacaaccgttggaggcgtccgccccgccaggtga

SEQ ID NO: 58 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNMRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 59 - DNA, 888 bp, synthetic construct
  source 1..888: mol_type = other DNA
  CDS 1..888: protein_id = 58; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactgtcccctctgtcccaacgttttgaacgtatcgcagttcagccgctgaccggtgtgctgggtgcagaaatcacgggcgtggatctgcgtgaaccactggatgactctacctggaacgaaatcctggacgcgttccacacttatcaggtgatctactttccgggtcaggcaatcaccaacgagcagcatatcgcgttcagccgtcgtttcggtccggttgaccctgttccgcacctgaaatctatcgaaggttacccggaagttcagatgatccgtcgtgaagcgaacgaaagcggtggcgtcatcggtgatgactggcacaccgatagcactttcctggacgcacctccagcagcagtggttatgcgtgcaattgatgttcctgaacacggtggcgacaccggtttcctgtctatgtataccgcctgggaaaccctgtctccgaccatgcaggctaccattgagggtctgaacgtcgttcacagcgcaacccgcgtgttcggctctctgtaccaggcgcagaacatgcgttggagcaacaccagcctgaaagttatggacgtagatgctggtgatcgtgaaaccgttcatccgctggttgttactcaccctggttccggccgtaagggtctgtatgtgaaccaagtgtattgccagcgtatcgaaggtatgaccgatgcagaaagcaaaccgctgctgcaatttctgtatgaacacgcaacccgtttcgacttcacttgccgcgttcgctggaaaaaagaccaggtactggtatgggataacctgtgtaccatgcaccgtgccgtaccggattacgcaggtaaattccgttatctgacgcgtaccaccgttggtggtgtgcgtccagcgcgttaa

SEQ ID NO: 60 - DNA, 888 bp, synthetic construct
  source 1..888: mol_type = other DNA
  CDS 1..888: protein_id = 58; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcccttagtcctttgagtcagagattcgagaggatcgccgtgcaacccctcaccggcgtgctcggagcggaaattacgggggtggacctgagagaacctttggatgactccacatggaacgaaatcctcgacgcgttccacacctatcaagtcatctacttcccgggtcaagcaattactaacgagcagcatattgctttcagcagacgcttcggacccgtcgaccccgtgccccatctcaagtcaatcgaggggtaccccgaggtccaaatgatcaggagggaggctaacgagtccgggggtgttatcggcgacgattggcacaccgattccacgttcctcgatgcgccaccggcagctgttgttatgcgggccatcgatgtccctgagcatggcggcgacacgggctttctttctatgtacaccgcttgggaaaccctttctccaaccatgcaagccacaatcgaaggtctcaacgtcgttcattctgccacgcgggtcttcgggagcctctatcaagcacagaacatgcgctggtcaaatacgtctctcaaggttatggacgttgacgccggtgatcgcgaaacggtccatccattggtggttacgcaccctggttccggaagaaaaggcttgtacgtgaatcaagtttattgccagcgcatagaggggatgaccgatgccgagtctaagccactgctccaatttctctacgagcatgcgaccagattcgatttcacgtgccgcgtgaggtggaagaaggaccaggtcctcgtgtgggacaatctctgcactatgcatagggcagtcccggactatgctggtaagttccgctacctcacccgcaccacagtcggcggcgttcgccctgcacggtga

SEQ ID NO: 61 - DNA, 888 bp, synthetic construct
  source 1..888: mol_type = other DNA
  CDS 1..888: protein_id = 58; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagccctttctcctctgtctcaacggttcgaacgaatcgccgtgcaacccctgactggggttttgggggcagaaataacgggtgtggatttacgtgaacctctcgacgattcgacgtggaatgagatactggatgcttttcacacgtatcaagtgatctatttcccagggcaagccataaccaacgaacagcacatcgccttttctagacgctttggcccggtagaccccgttcctcacttaaaatcaatagaaggttatcccgaagtacagatgattcgtagagaagccaatgaatcgggcggagtaataggggacgattggcacacggacagcacatttcttgatgctccgcccgccgccgtggtaatgcgcgctattgatgtacctgagcatggcggtgatacaggctttttaagcatgtacaccgcgtgggagactctgagtccaactatgcaagctacaatcgaagggctcaatgtcgttcactctgcgacacgggtgtttggctccttatatcaggcccagaatatgcgctggtcaaatacgtcacttaaagtaatggacgtcgatgcgggggatcgcgagactgtccaccctctggtagttacgcacccggggtcaggaaggaaaggcttgtacgtgaatcaagtctattgccagaggatcgagggtatgactgatgctgaatcaaaacccctcctacaatttttgtacgagcatgcgacgcgattcgatttcacttgcagggttaggtggaaaaaagaccaggtcctagtatgggataatctgtgtacaatgcatcgcgcagtccctgactatgcggggaagtttcgatatctgactagaaccacggtagggggagtgcgaccggcgcgatga

SEQ ID NO: 62 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGCDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 63 - DNA, 888 bp, synthetic construct
  source 1..888: mol_type = other DNA
  CDS 1..888: protein_id = 62; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGCDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgtctcctctgtctcagcgtttcgaacgtatcgcagttcagccgctgaccggcgttctgggtgctgaaatcactggcgttgacctgcgtgagccgctggacgattctacctggaacgaaattctggatgctttccacacgtatcaggtaatctactttccgggccaggctatcactaacgaacagcacatcgcgttctcccgtcgttttggcccggttgacccggttccgcatctgaaatccatcgagggttacccggaagtccagatgatccgtcgtgaagcgaacgaatctggtggtgtgatcggttgcgactggcacactgatagcactttcctggatgcgccgccggcagctgttgttatgcgtgctattgacgttccagaacacggtggcgatactggctttctgtccatgtataccgcatgggagaccctgtctccgactatgcaggcgacgattgagggtctgaacgtcgttcactctgcaacgcgtgtattcggttccctgtaccaggcgcagaaccgtcgttggagcaacacctctctgaaagttatggatgtggatgcaggtgaccgtgagaccgtgcatccgctggtagttactcacccgggttctggccgcaaaggcctgtatgttaaccaggtgtattgccagcgcatcgaaggtatgaccgacgctgagtctaaaccgctgctgcagttcctgtatgaacacgcgactcgtttcgacttcacctgtcgcgttcgctggaaaaaagaccaggtgctggtctgggataacctgtgcaccatgcatcgtgctgtgccggactacgcaggtaaattccgttatctgacccgcactaccgtgggcggtgttcgtccagctcgttaa

SEQ ID NO: 64 - DNA, 888 bp, synthetic construct
  source 1..888: mol_type = other DNA
  CDS 1..888: protein_id = 62; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGCDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctttccccactcagccaacgttttgagcggattgctgttcagcctcttaccggcgtcctgggtgccgaaatcactggtgtggacctcagggagccattggacgactcgacctggaatgagattctggacgcgtttcatacctaccaagtgatctatttcccaggccaggccattaccaacgagcagcacatcgcctttagcaggaggtttggtccggtggacccggtcccacatctcaaatcaattgaaggctatcccgaggtgcagatgatccggagggaagcaaacgagagcggcggtgtgatcggctgcgattggcatactgacagcaccttcttggatgcacctcccgccgcagtggtcatgagagctatcgacgtccccgagcatggtggcgataccggattcctgtccatgtacaccgcatgggaaacgctctcacctacgatgcaggctaccatcgaagggctcaacgtggtgcatagcgccaccagggtgtttggctcactgtaccaggcgcaaaaccgccgctggtctaacacttctctcaaagtgatggatgtcgacgccggtgatcgtgaaacggtccatcctctcgtggttacccaccctggctcgggtagaaaggggctttacgtgaaccaggtctactgtcagaggatcgagggaatgaccgacgctgagtccaagccgctgttgcaattcttgtacgagcatgctacgagattcgacttcacttgtcgcgtgaggtggaagaaggatcaggtcctggtgtgggacaatctgtgtactatgcatcgcgcggttccagactatgccggcaagttcagatatctgactagaactacggtcggcggagttaggcctgctaggtga

SEQ ID NO: 65 - DNA, 888 bp, synthetic construct
  source 1..888: mol_type = other DNA
  CDS 1..888: protein_id = 62; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGCDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgccctatctcccctaagtcagcgctttgaacgtattgccgtacagccccttactggagtgcttggcgctgaaatcacgggggtggacctgagggaaccgttggatgattctacatggaacgagattttggatgcttttcacacataccaggtcatatactttcctggacaagcaatcaccaatgagcagcacatagctttctcccgtcgttttggacccgtcgatcctgtgccgcatttgaaatctatcgagggatatcctgaggttcaaatgatccggagggaagccaatgaaagtgggggagtaatcggatgtgactggcatacagattcgactttcttagacgcacccccggctgcagtagtaatgagagcaatagatgtacctgagcacgggggggacacagggtttctgagcatgtataccgcctgggagacactgagtcctaccatgcaggcaaccatagagggacttaacgtagtgcactccgctacgcgtgtttttggctctttgtaccaggctcagaataggcgatggagcaatacgtccctcaaagttatggatgtcgatgcgggtgacagagaaacagtgcacccgctcgtcgtaactcatccgggctccggtcgaaagggactgtatgtgaaccaagtatactgtcagcgcattgagggaatgacagatgcggaaagcaaaccccttttgcaattcctttacgaacacgctacgcgttttgacttcacctgtagagtgcgctggaagaaagatcaggtgttggtatgggataatttgtgcactatgcatagagctgtacccgactacgccggcaagtttcgttacctaacccgcacgacggttggtggagtacgtcctgcgcggtga

SEQ ID NO: 66 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDEWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 67 - DNA, 888 bp, synthetic construct
  source 1..888: mol_type = other DNA
  CDS 1..888: protein_id = 66; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDEWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcgctgtctccgctgtctcagcgttttgaacgtattgctgtccagccactgaccggtgttctgggtgcggaaatcacgggtgttgatctgcgcgaaccgctggatgattccacctggaacgaaatcctggatgcattccatacctaccaggtgatctacttcccgggccaggcaattaccaacgaacagcatattgctttttcccgtcgtttcggtccggtagatccggtgccgcatctgaaatctatcgagggctacccggaagttcagatgattcgtcgcgaggctaacgaatccggtggtgtcatcggtgatgaatggcacactgactctacctttctggatgcaccaccggctgcggttgttatgcgtgcgattgatgtaccggaacatggtggtgacactggcttcctgtctatgtatacggcttgggaaactctgtccccgaccatgcaagccaccattgaaggcctgaacgtggttcactccgcgactcgtgtgtttggctctctgtaccaggctcagaaccgtcgttggtctaacacttctctgaaagttatggacgtagatgcaggcgatcgtgagacggtacacccactggtagttactcacccgggtagcggtcgtaaaggcctgtacgttaatcaggtatactgccagcgtatcgagggtatgaccgatgcagaatctaaaccgctgctgcagtttctgtacgaacatgccacccgttttgatttcacttgtcgtgttcgctggaagaaagaccaggttctggtttgggacaacctgtgtaccatgcatcgtgcggtgccggactatgcgggtaaattccgctacctgactcgcaccactgtgggtggtgtacgtccggctcgttaa

SEQ ID NO: 68 - DNA, 888 bp, synthetic construct
  source 1..888: mol_type = other DNA
  CDS 1..888: protein_id = 66; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDEWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggcattgtccccactctcacagagatttgagcgcatcgccgttcaaccgctcacaggtgtgttgggagccgagattacaggggtggatttgagggagcccctcgacgacagcacatggaatgagatcttggatgcattccatacgtaccaggtgatctacttccccgggcaagctatcaccaatgagcagcacatcgcgttcagtcgtcgtttcggacctgtcgatcctgttccacacctgaaaagcatcgagggctaccccgaggtgcaaatgattcggcgcgaagccaatgagtccggcggagttatcggagacgagtggcacactgacagtactttcctggatgctccaccagcagctgtcgttatgagagctatcgacgttccagagcatggtggcgataccggcttcctgtcaatgtacactgcctgggaaacccttagcccaactatgcaagctaccatagaggggctgaacgtcgtccattcagcgacacgcgttttcggatctctgtatcaggctcagaaccgccgctggagtaacactagcctgaaggttatggatgttgatgccggggatcgggagactgttcaccctctcgtggttacccacccgggttcaggaaggaaggggctttacgtgaaccaggtctattgccagcggatcgagggaatgactgacgctgagtctaagccgctcctccaattcctctatgagcacgcgacgcgcttcgacttcacgtgtcgcgtgcgctggaagaaggaccaagttctcgtgtgggacaacttgtgtacgatgcacagagcggtccccgactacgctggcaaatttaggtatctcaccagaacaacggtgggcggggtgagacctgcaaggtga

SEQ ID NO: 69 - DNA, 888 bp, synthetic construct
  source 1..888: mol_type = other DNA
  CDS 1..888: protein_id = 66; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDEWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcggcattatctccgttatcacagcgctttgaaaggatagcagtccaaccactaacaggcgtccttggtgctgaaataacaggggttgatctcagggaacccctggacgactcgacctggaacgagattctggacgcgttccatacttatcaagtaatctattttccagggcaggcaatcacgaatgagcagcacatagcattttcacgcagatttgggcccgtagatccagttccgcatttaaagtctatcgagggatatcctgaggtccagatgatcagacgggaagctaacgagtctgggggggtcattggagacgaatggcacaccgatagcaccttcctcgatgcgccacccgcagctgtggtcatgcgagcaatcgatgtgcccgaacatgggggcgatacagggttcctaagtatgtataccgcctgggaaactttatctcctacgatgcaagcaactatcgaaggacttaacgtcgttcactcagcgactcgtgtgttcggaagcttgtatcaagcgcaaaaccgccgatggagtaatacatctcttaaagtcatggatgttgacgctggggacagagaaactgtgcatcctttagtggtgactcatcctggaagcggacgtaaaggtttatacgtaaaccaggtatactgccagagaatcgaaggcatgaccgatgccgaaagcaaacccttactacaattcctctatgaacatgcaaccaggttcgatttcacatgccgagttcggtggaaaaaagaccaagtccttgtatgggataatctatgtactatgcatagagccgttccagattacgccggtaagtttaggtacctgacgcggactacagtaggaggagtacgcccagccagatga

SEQ ID NO: 70 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNCVYCQRIEGMTDAESKPLLQFLYEHATRFDFTCRVRWKKDQVLVWDNLCTMHRAVPDYAGKFRYLTRTTVGGVRPAR

SEQ ID NO: 71 - DNA, 888 bp, synthetic construct
  source 1..888: mol_type = other DNA
  CDS 1..888: protein_id = 70; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN CVYCQRIEGMTDAESKPLLQFLYEHATRFDFTCRVRWKKDQVLVWDNLCTMHRAVPDY AGKFRYLTRTTVGGVRPAR
atgcatgctgcgctgtctccactgtcccaacgttttgaacgtatcgcagttcaaccgctgaccggcgtgctgggtgctgaaattaccggcgtagatctgcgtgaaccgctggacgattctacttggaacgagatcctggatgcgtttcacacttaccaggttatctatttcccgggccaggccatcacgaacgaacagcatatcgcattctctcgtcgctttggcccggtggatccggttcctcacctgaaaagcattgaaggctacccggaggttcagatgatccgtcgtgaagcaaatgaatccggcggtgttatcggtgatgactggcacactgactccactttcctggacgctccgccggcagctgtagttatgcgtgcgatcgacgttccggaacacggtggtgacaccggcttcctgtctatgtacactgcatgggaaaccctgagcccgaccatgcaggcaaccatcgaaggtctgaacgttgttcactccgctacccgtgtatttggttccctgtaccaggcacagaatcgtcgttggtccaacacttctctgaaagtgatggacgtggacgcgggcgatcgtgaaaccgttcatccgctggtagttacccatcctggttctggccgtaaaggcctgtacgtaaactgtgtatactgccagcgcattgaaggcatgaccgacgcggaatctaagccgctgctgcagttcctgtatgaacacgcgactcgtttcgattttacctgccgtgtgcgttggaaaaaagatcaggttctggtatgggataacctgtgcaccatgcatcgtgcggttccggattacgcgggcaaatttcgttatctgactcgtactacggttggtggcgttcgtccggcacgttaa

SEQ ID NO: 72 - DNA, 888 bp, synthetic construct
  source 1..888: mol_type = other DNA
  CDS 1..888: protein_id = 70; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN CVYCQRIEGMTDAESKPLLQFLYEHATRFDFTCRVRWKKDQVLVWDNLCTMHRAVPDY AGKFRYLTRTTVGGVRPAR
atgcatgccgctttgtcgcctctctctcagcgttttgaaaggatcgccgttcaacctttgaccggcgtgcttggggctgagataacaggtgtcgacttgcgcgaaccgcttgatgactcaacttggaacgagatcctcgatgcttttcatacttatcaggtcatttacttccccggacaagcgatcacaaatgaacaacacatcgcgttctcccgccgcttcggccctgtggaccctgttccccatttgaagtccattgagggttaccctgaagtgcagatgattcgcagggaggccaacgagtctggcggcgtgattggagatgattggcacacggatagcacgtttcttgacgcgccaccagctgccgtggtgatgagagccatcgacgtgccggagcatggtggagacactggattcctctcgatgtacacggcgtgggagactctctcaccaacaatgcaggcgaccattgagggcttgaatgtggtgcacagcgccacgcgcgtcttcggttccctgtaccaagcgcaaaatcgccgctggtcaaatacctcgttgaaggtgatggatgtggatgccggagatagggagaccgtccaccccctcgttgtgactcaccctggctccgggaggaagggtctctacgtgaactgcgtgtattgccagaggatagaggggatgaccgatgcggagtctaagccactcttgcaattcctttacgagcatgctacgcggttcgacttcacctgccgggttaggtggaagaaggaccaggtcctcgtctgggataatctctgtaccatgcacagagctgtgccagactacgcgggaaagttcaggtacttgacacgtaccaccgtgggcggggtcagaccggctcgctga

SEQ ID NO: 73 - DNA, 888 bp, synthetic construct
  source 1..888: mol_type = other DNA
  CDS 1..888: protein_id = 70; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN CVYCQRIEGMTDAESKPLLQFLYEHATRFDFTCRVRWKKDQVLVWDNLCTMHRAVPDY AGKFRYLTRTTVGGVRPAR
atgcatgcagcattgagtcccctaagccagcgctttgagaggattgcagttcaaccgctcacaggggtattaggtgcagaaatcacaggagtcgatttacgggagcccctagatgatagcacgtggaacgaaattcttgacgccttccacacttatcaggttatttattttccaggacaagcgataacgaacgagcagcatatagcattttccagaaggtttgggcccgtagacccggtgccccacctgaagagcattgagggataccccgaagtccagatgatacgcagagaggcgaacgagagcggaggtgttataggtgacgattggcatacagactcaacgtttttagacgccccccctgcggccgtggtgatgcgggcaatcgacgttccggaacatggtggggacactggcttcctttccatgtacaccgcgtgggaaacgctctcccccaccatgcaggctaccattgaagggcttaatgtggtacattccgcgacacgtgtcttcggaagtttgtaccaggcccaaaatcgacgatggagcaatactagccttaaggtaatggacgttgatgcgggcgatagagaaacagtccaccctcttgtcgtcacacatccaggttccgggcgtaagggcctttacgtcaactgcgtttattgccaaaggattgaaggcatgaccgatgcggagtcaaaaccgctgcttcagttcctttatgaacacgctacgaggtttgactttacttgccgtgtgcgctggaaaaaagatcaagtactggtctgggacaatctgtgtactatgcaccgagcagttccggactatgccgggaaattcagatatttgactagaacgacagtcggcggcgtgcgcccagccagatga

SEQ ID NO: 74 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNPVYCQRIEGMTDAESKPLLQFLYEHATRFDFTCRVRWKKDQVLVWDNLCTMHRAVPDYAGKFRYLTRTTVGGVRPAR

SEQ ID NO: 75 - DNA, 888 bp, synthetic construct
  source 1..888: mol_type = other DNA
  CDS 1..888: protein_id = 74; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN PVYCQRIEGMTDAESKPLLQFLYEHATRFDFTCRVRWKKDQVLVWDNLCTMHRAVPDY AGKFRYLTRTTVGGVRPAR
atgcacgctgctctgtccccactgtctcagcgttttgaacgcattgcagttcagccgctgactggcgtactgggtgcagaaatcaccggtgttgatctgcgtgaaccgctggacgacagcacttggaacgaaatcctggatgcatttcatacttaccaggttatctacttcccgggccaggccatcacgaacgaacaacatatcgccttcagccgtcgtttcggtccggttgacccggtaccgcacctgaagtccattgagggctatccggaagttcagatgattcgccgcgaagcgaacgagtctggtggcgttatcggtgatgactggcacaccgactctacgttcctggacgccccgccagcagcagttgttatgcgtgcgattgatgtgccggaacacggtggtgacactggtttcctgagcatgtataccgcttgggaaaccctgagcccaaccatgcaggcaaccatcgaaggcctgaacgttgtgcacagcgctacccgcgttttcggcagcctgtatcaggcacagaaccgtcgttggagcaacacttctctgaaagttatggacgttgacgctggcgatcgtgaaaccgtacaccctctggttgttacccacccgggctctggccgtaaaggtctgtatgtgaacccggtgtattgccagcgtatcgaaggtatgaccgatgcagaatccaaaccgctgctgcagttcctgtatgaacacgcgacccgcttcgacttcacgtgtcgtgtacgttggaaaaaagaccaggttctggtctgggacaacctgtgtactatgcatcgtgcagttccagattacgcaggcaagtttcgttatctgactcgtaccactgtaggtggtgtgcgtcctgcacgttaa

SEQ ID NO: 76 - DNA, 888 bp, synthetic construct
  source 1..888: mol_type = other DNA
  CDS 1..888: protein_id = 74; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN PVYCQRIEGMTDAESKPLLQFLYEHATRFDFTCRVRWKKDQVLVWDNLCTMHRAVPDY AGKFRYLTRTTVGGVRPAR
atgcacgctgcgctctctcccctttcccaaagattcgagaggattgctgtgcaacctcttacgggggttcttggtgcagagatcacaggcgttgatctccgcgagccccttgacgactcaacctggaatgagatacttgacgcgtttcacacataccaggtgatttacttcccgggccaagctatcaccaacgaacagcacattgctttcagtcgtcgttttggcccagtcgacccagtcccgcacctcaagagcattgaagggtacccagaggtccagatgattcgccgcgaggcgaatgagtcaggcggcgtgatcggcgacgactggcatactgactctacttttctggacgcgccccctgccgctgttgttatgagggcaatcgatgtgccggagcacggaggcgatactggcttcctctccatgtacacggcatgggagaccctgtcccctaccatgcaggccaccatcgaaggtctgaacgttgtccatagcgcgacccgcgtgttcggctctctctaccaggcccagaatagacgttggagcaataccagcctcaaggtcatggacgtcgacgcaggggaccgggagaccgtccacccactcgttgttacgcacccaggaagcggacgcaaagggctctacgtgaacccggtgtattgtcagaggatcgagggcatgacagatgccgaaagcaagcctctcctccagttcctctatgaacatgccaccaggttcgatttcacatgcagggtccgctggaagaaggaccaagtccttgtttgggataatttgtgtacgatgcacagggccgttcccgactacgctggtaagttccggtacttgacccgcaccaccgtgggcggcgtcaggccagcacgctga

SEQ ID NO: 77 - DNA, 888 bp, synthetic construct
  source 1..888: mol_type = other DNA
  CDS 1..888: protein_id = 74; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN PVYCQRIEGMTDAESKPLLQFLYEHATRFDFTCRVRWKKDQVLVWDNLCTMHRAVPDY AGKFRYLTRTTVGGVRPAR
atgcatgccgccctttctccgctatctcagcgttttgagaggatagccgtgcaacccctgactggagtattaggcgctgaaatcaccggcgtggacctaagagaaccattagatgattcgacttggaatgagatactggatgcgtttcatacttaccaggtaatttattttcccggacaagccatcacgaacgagcaacacattgcattctctcgacgttttggcccagttgatcccgtgccccatttgaaatcaatagaaggttaccccgaagtgcaaatgattcgtagggaggcaaacgaaagtggaggagtcatcggcgacgactggcacactgattcaacatttcttgacgcaccaccagccgcagttgtaatgagggccattgatgttccggaacatgggggagatacggggtttttgtcaatgtataccgcgtgggaaacgttatctccgacaatgcaggcaaccattgaaggcttgaatgtggtccattctgctaccagggtgttcggatctttgtatcaagctcaaaacagaaggtggtccaacacttcgctgaaagtaatggatgtagatgctggagaccgagaaacggttcacccattggtggtaacgcatccaggatcggggaggaagggattgtatgtaaaccctgtgtactgccagagaatcgagggaatgactgacgccgaatcgaaaccgttgttgcagtttctatacgagcatgcaacaaggttcgatttcacctgccgagtgcgctggaagaaagaccaggttttagtgtgggataatctatgcactatgcatcgggctgtgcctgactatgccggaaaattcagatacttgacacgcacaaccgtcggtggagtacgaccagcaaggtga

SEQ ID NO: 78 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNDRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 79 - DNA, 888 bp, synthetic construct
  source 1..888: mol_type = other DNA
  CDS 1..888: protein_id = 78; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNDRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctctgtctccgctgtctcagcgttttgaacgtatcgcggtacagcctctgactggcgttctgggcgcagagatcaccggcgtagatctgcgcgaaccgctggacgattctacctggaacgaaattctggatgctttccacacctaccaggttatttactttccgggtcaggcaattaccaacgagcaacatatcgctttctcccgccgttttggtccagttgatccggttccgcacctgaaatccatcgaaggttacccggaggttcaaatgattcgtcgtgaagcgaacgaaagcctgcgcgtgatcggtgatgactggcacaccgatagcacctttctggacgcgccgccagcagctgttgttatgcgtgctatcgacgttccagaacacggtggtgacaccggtttcctgtctatgtacaccgcgtgggaaaccctgtccccgaccatgcaggcgactattgaaggcctgaacgttgtacactctgcaacccgcgtgttcggttctctgtaccaggcgcagaacgaccgttggagcaacacctctctgaaagtgatggatgttgacgcaggcgaccgtgaaacggtgcatccgctggtagtaacccatcctggctctggtcgtaaaggtctgtacgtcaaccaagtttactgccagcgtatcgaaggtatgaccgatgctgaatccaaaccgctgctgcaatttctgtatgaacacgctacccgcttcgacttcacctgtcgcgttcgttggaaaaaagatcaggttctggtatgggacaacctgtgcaccatgcaccgtgccgtgccggattacgcaggtaaattccgctacctgacccgtactaccgttggcggcgttcgtcctgcacgttaa

SEQ ID NO: 80 - DNA, 888 bp, synthetic construct
  source 1..888: mol_type = other DNA
  CDS 1..888: protein_id = 78; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNDRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgccctcagcccattgagccagcgtttcgaaagaatagccgttcagcctttgaccggcgtgctcggcgctgaaattactggtgtggacctgcgcgagccactggacgactccacctggaatgagatcttggatgctttccatacctaccaagtcatttactttccagggcaggccatcaccaatgagcaacacatcgcgttcagcaggcgcttcggacccgtcgacccggtcccccatcttaagtccatcgaaggctacccggaggtccaaatgatacgcagagaagctaatgagtctttgagagtcataggcgatgattggcacaccgattctacgttccttgacgccccgccggccgccgtggtcatgcgggccattgatgtgcctgagcacggtggcgacacaggcttcttgtcaatgtacacagcctgggaaacattgagcccaaccatgcaggcgacaatcgaagggttgaatgtggtgcattctgcgaccagagtgttcgggtcgctgtaccaggctcagaatgatcgctggtctaacacatccctgaaggtcatggatgtcgatgccggggaccgtgagacagtccatccgcttgtcgtcacgcaccccgggagcggccgcaaggggctgtatgtcaatcaggtgtactgccagcgcatcgaagggatgaccgacgccgaatccaagcccctcctgcaattcttgtatgagcatgcaacaaggtttgacttcacgtgccgggttagatggaaaaaagatcaagtgctcgtgtgggacaacctctgcacaatgcatagggccgttcctgactacgcgggcaagtttcggtacttgacgagaacaaccgttgggggggtgcgtcctgcccgctga

SEQ ID NO: 81 - DNA, 888 bp, synthetic construct
  source 1..888: mol_type = other DNA
  CDS 1..888: protein_id = 78; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNDRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgccttgtctcccttatcacaacgttttgaaaggatagcagttcaacctcttacgggagttttaggtgccgagataacgggggtcgatttgcgcgagccgctcgacgatagcacttggaatgaaattctggatgcctttcacacataccaagtcatctatttccccggtcaggctattacaaatgaacaacatatcgcttttagccggcgctttggccctgtagatcccgtcccccacttgaagagtatcgagggctacccggaggtacagatgatccgacgtgaagccaacgaatccctaagagtgattggagacgattggcacacggattcaacctttcttgatgccccgccggcagcagttgtaatgagggcaattgatgttccagagcacggaggagatacaggtttcttgtcgatgtataccgcctgggaaaccttatcacctacgatgcaagcaacaatagaagggctcaatgtcgttcactcagcgacgcgggttttcgggtctctctaccaggcccagaatgatcgctggagcaatacaagcctgaaagtgatggatgtagatgcaggagaccgtgaaacggtacatcctttggtagtgacacacccgggcagcggtcgtaaagggctatatgttaaccaagtgtattgccagagaatagaagggatgaccgatgcagaatcaaaaccactcttacaattcctgtatgaacacgcgacgagatttgattttacatgcagggtcaggtggaaaaaggatcaggtgttggtgtgggataatctttgcactatgcatagagccgtccccgattatgccgggaaatttcgttatcttacacgaaccactgtaggcggagtccgcccggcccgatga

SEQ ID NO: 82 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNERWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 83 - DNA, 888 bp, synthetic construct
  source 1..888: mol_type = other DNA
  CDS 1..888: protein_id = 82; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNERWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactgtctccactgtcccagcgtttcgaacgtattgctgttcagccgctgactggtgtactgggtgcggaaatcaccggcgttgacctgcgtgaaccactggacgacagcacgtggaacgaaatcctggacgcgttccacacctaccaggtgatttattttccgggtcaggcaatcaccaacgaacagcacatcgccttcagccgccgttttggtccagttgatccggttccgcatctgaaatccatcgaaggctacccggaagttcagatgattcgtcgtgaagcgaatgaatctctgcgtgtgatcggcgacgattggcacactgattctacctttctggacgctccaccggctgcagttgtaatgcgcgcaatcgatgttccggagcacggtggtgacactggttttctgtctatgtataccgcatgggaaaccctgtccccgaccatgcaggcaaccattgaaggcctgaacgtggttcactctgctacccgtgtctttggctccctgtaccaggcgcagaacgagcgttggtctaacacgagcctgaaagtgatggatgttgatgcgggcgaccgtgaaaccgttcatccgctggtcgttactcacccgggctctggtcgcaaaggcctgtatgttaaccaggtttattgccagcgtatcgaaggcatgaccgatgcggaatctaaaccgctgctgcagttcctgtacgaacacgctacccgttttgacttcacctgccgtgttcgctggaaaaaggatcaggtgctggtttgggataacctgtgcaccatgcaccgcgctgttccggactatgcaggtaaattccgctacctgacccgtactactgttggtggcgttcgcccagcccgttaa

SEQ ID NO: 84 - DNA, 888 bp, synthetic construct
  source 1..888: mol_type = other DNA
  CDS 1..888: protein_id = 82; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNERWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctctttctccgctgtctcagagattcgagaggatagccgtgcagccactgaccggggttcttggagccgagattacaggcgtcgatttgagggagccacttgacgactccacatggaacgaaatcctggacgcctttcatacgtaccaggttatctacttccccggccaggctatcacgaacgagcagcacatcgcgttttctcggcgtttcggcccagtggacccggtcccacatttgaaatcgatcgagggctaccccgaggtgcaaatgattagaagagaagcgaacgagtctctccgcgttataggcgatgattggcatacagattcgaccttcttggatgcaccaccggcagccgtcgtgatgcgcgccattgacgttccggagcacggaggcgataccgggttcctctcaatgtacacagcctgggaaacgctttctccaacaatgcaagcgacgattgaaggactgaatgtcgtgcattcagctactagggtcttcgggtccctctaccaggcccagaatgagagatggtcgaatacgtcgttgaaggttatggatgtggatgctggcgaccgcgagaccgtgcaccctctggttgtgacccatccgggtagtggtcgcaagggcctctacgtgaaccaggtttactgccagagaatcgagggaatgactgatgccgagtcgaagccactcctccagttcctctacgagcacgccacaaggttcgacttcacctgtcgtgtgcggtggaagaaagaccaggtcctcgtgtgggataatctctgtactatgcacagagccgtgcccgactacgccggcaagttccggtatctcacgaggacgacagtcggtggcgtgcggcctgccagatga

SEQ ID NO: 85 - DNA, 888 bp, synthetic construct
  source 1..888: mol_type = other DNA
  CDS 1..888: protein_id = 82; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNERWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcgctcagccccctatcccagcgctttgaaagaatagcagtccaaccactgacgggggtattaggcgccgaaattaccggtgtggatttaagagaaccgttggatgatagcacctggaatgaaattctcgatgcttttcacacctaccaggtaatttactttccaggccaagctataactaacgaacagcacatcgcattcagtagacgtttcgggccagttgatccggtgccacaccttaagtccatcgaaggctaccctgaggtccagatgatacgccgagaggccaacgaaagcttacgtgtgatcggggacgactggcatacagatagcaccttccttgacgcaccccccgcagccgtcgttatgcgtgccatcgacgtcccagagcatggcggagacactggatttttatctatgtataccgcatgggaaacgttaagccccaccatgcaagcgaccatagagggattaaacgtggtccattcggctacacgggtgtttggatccttgtatcaggcacagaatgaacgttggagtaatacctcacttaaagtcatggatgtggatgctggggacagggagacagttcacccgttggttgtcacgcatcctggatctgggcgcaaaggactttacgtaaaccaagtatattgtcaacgtatagaagggatgacggacgccgagagcaaacctttactccaattcctgtatgagcacgcgacgagatttgactttacttgtcgtgtcagatggaaaaaagatcaggtcttagtttgggacaatctttgcacgatgcacagagccgtgccagattatgctggtaaatttagatacctgacgcgaaccaccgtcggcggcgttcggccagcgaggtga

SEQ ID NO: 86 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNSRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 87 - DNA, 888 bp, synthetic construct
  source 1..888: mol_type = other DNA
  CDS 1..888: protein_id = 86; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNSRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctctgtctccgctgtctcagcgtttcgaacgtattgctgttcagcctctgaccggtgttctgggcgcagaaatcactggcgttgacctgcgtgaaccgctggacgattctacctggaacgagattctggatgctttccacacttaccaagttatttacttcccaggtcaagcgattactaacgaacaacacattgccttctctcgtcgtttcggtccagttgatccggtcccacacctgaaatctatcgaaggttacccggaagtacaaatgatccgtcgcgaagcaaacgaatccctgcgtgtaatcggtgatgattggcacaccgacagcaccttcctggatgcaccgccggcggctgttgttatgcgtgctatcgatgttccggaacatggcggtgatactggcttcctgagcatgtacaccgcatgggaaacgctgtctccgaccatgcaggcaaccatcgaaggcctgaacgtggtacattctgcgactcgcgtattcggctccctgtatcaggcacaaaactctcgttggtccaacaccagcctgaaagtaatggacgttgatgctggtgaccgcgaaaccgtacacccactggtggttacccacccaggtagcggtcgtaaaggtctgtacgttaaccaggtttattgtcaacgtattgaaggtatgactgatgcagaatccaaaccgctgctgcagttcctgtacgaacacgcgacccgtttcgattttacttgccgcgttcgttggaaaaaagaccaggttctggtgtgggataacctgtgcactatgcaccgcgcagttccggactacgcaggtaaattccgttatctgactcgtaccactgttggtggtgttcgtccggcacgttaa

SEQ ID NO: 88 - DNA, 888 bp, synthetic construct
  source 1..888: mol_type = other DNA
  CDS 1..888: protein_id = 86; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNSRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctctcagccctctttcccaaaggttcgaacggatcgccgtgcagcccctgactggtgttctcggagcggagattaccggcgtcgatctccgcgagcccttggatgacagcacatggaatgagattctcgacgcgttccacacttaccaagttatctatttcccgggccaggctatcaccaacgagcagcacatcgccttctctaggagatttggcccagtggacccagtcccacatctgaagtcgatcgaaggctacccagaggtccagatgattaggagggaagcaaacgaatctcttcgcgtcatcggcgacgactggcatactgattcgacgtttctcgacgctcctccagcggctgttgtgatgcgtgctatcgatgttcctgagcatggcggcgacactggcttcctgtccatgtacacggcctgggagaccctctccccaacaatgcaggctaccattgagggactcaatgtcgttcactcggcgacacgtgtgttcggcagcctttatcaggctcagaactccaggtggagcaacacaagtctgaaggtcatggacgtcgacgcgggtgacagagagacagtgcaccctcttgtggtgacacatcctggctccggtcgtaaagggctgtacgtgaaccaggtttactgtcaaagaattgaagggatgaccgatgcggagtccaagccccttttgcagtttctgtatgagcatgcgaccagattcgatttcacttgtagggtgagatggaagaaggaccaggtgctggtgtgggataatttgtgtacgatgcatagggcagtgcccgactatgctggtaagtttaggtacctcacccggactacagtcggaggagtcagaccagcgcggtga

SEQ ID NO: 89 - DNA, 888 bp, synthetic construct
  source 1..888: mol_type = other DNA
  CDS 1..888: protein_id = 86; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNSRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcgctgagccccctcagtcaacgatttgagagaattgctgtacagccattaacaggcgttctgggagcagaaataacaggagttgatcttcgtgaacccctagacgattccacatggaacgaaattctggatgcgttccacacgtaccaagtcatctacttcccgggccaagcgatcacaaatgaacagcatatagcgttttcgaggcgattcggaccagtagatcctgtgccccatttaaagtcgatcgaagggtatccagaagtccaaatgatacgcagggaggctaacgaatcattacgggtcattggagatgactggcatacggattcgacgtttcttgatgcgccgccggcggcggtagtcatgcgagcgatcgatgttcctgaacacggaggcgatacgggcttcctgtccatgtacaccgcatgggaaacgttgtctcccacaatgcaagcgaccatcgaaggacttaatgttgtacattctgcgacaagggtattcggatctctgtaccaggctcaaaattcgcgttggtctaacactagcctcaaagtgatggatgtcgacgcaggtgaccgcgagaccgttcaccctctagtcgtaacccatccggggagtggtcgtaaaggactctatgtgaaccaagtgtactgccagagaatagagggaatgacggacgctgagtctaaacctctcctccagttcctgtacgagcacgctacccgctttgacttcacatgccgcgtccgatggaaaaaagatcaagtgttagtatgggataacctctgcactatgcatagagcagtacctgattatgccggcaagttccggtacctaacccgcacaactgtaggcggagtgagacctgcccggtga

SEQ ID NO: 90 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNLRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 91 - DNA, 888 bp, synthetic construct
  source 1..888: mol_type = other DNA
  CDS 1..888: protein_id = 90; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNLRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctctgtctccgctgtctcagcgtttcgaacgtattgcagtacagccactgaccggtgtgctgggcgcagaaattaccggcgtagatctgcgtgaaccgctggacgacagcacctggaacgaaattctggatgcgttccatacttaccaggttatctacttcccaggtcaggccatcacgaacgagcagcatatcgcgttctcccgtcgtttcggtcctgttgacccggtcccgcacctgaaatctatcgaaggctatccggaagtgcagatgatccgtcgtgaagcaaacgaatctctgcgtgtaattggtgacgattggcacaccgactctacttttctggatgcaccgccggccgctgttgtaatgcgtgctattgacgttccggaacatggcggtgataccggtttcctgtctatgtatacggcgtgggaaaccctgagcccgaccatgcaggccactatcgaaggcctgaacgtggttcactctgctacccgtgtttttggctccctgtatcaggcgcagaacctgcgctggtccaacacctctctgaaagtcatggacgttgatgctggtgatcgcgaaactgtgcaccctctggttgttactcacccgggttctggccgtaaaggcctgtacgtgaaccaggtttattgccagcgtatcgagggcatgaccgacgctgaatctaaaccgctgctgcagtttctgtatgaacacgcaacccgctttgactttacctgccgtgttcgttggaaaaaagatcaggttctggtatgggataacctgtgcactatgcatcgtgccgtgccagattacgccggtaaattccgttatctgactcgtaccaccgttggtggcgttcgtccggctcgttaa

SEQ ID NO: 92 - DNA, 888 bp, synthetic construct
  source 1..888: mol_type = other DNA
  CDS 1..888: protein_id = 90; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNLRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggccttgtctccactctctcaacggtttgaacgcattgcggtccaacccctcaccggcgttttgggggccgaaatcaccggcgtggacctcagagaaccacttgacgattcaacttggaacgaaatcttggacgcatttcacacttaccaggtcatttactttccggggcaggcgattacaaacgaacaacacatcgccttctcaagaaggttcggtcctgtcgacccagttccacacctcaagtccatcgagggctacccggaggtccagatgattcgtcgcgaggctaacgagtcgttgcgcgtcattggagacgattggcacactgactctacctttctggacgcacctccggctgccgtcgtgatgcgggctatcgacgtgccggagcacggcggggatacaggctttctcagcatgtacaccgcatgggaaacactctcacctacgatgcaggccaccatagagggcctcaacgtcgttcacagcgctacgcgggtgtttggttctttgtaccaggcgcagaaccttcgctggtctaatacgtctctcaaagtgatggacgtcgatgccggagaccgcgagactgtgcaccccctggtggtgacccatcccgggtcgggacgcaagggactgtacgttaaccaagtgtactgccagaggatagagggcatgactgatgccgagagcaagccgctgctccaattcctctacgagcacgcgacaaggttcgatttcacttgccgggtgcgctggaaaaaggaccaggtgctcgtgtgggacaacctgtgcaccatgcatagggctgtcccggactatgccggcaagttccggtatttgactaggacaacggtcggaggagttaggcctgcgcgctga

SEQ ID NO: 93 - DNA, 888 bp, synthetic construct
  source 1..888: mol_type = other DNA
  CDS 1..888: protein_id = 90; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNLRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgccgccctgtctccattaagtcaacgattcgaacgcatagcggtacagcccttgaccggtgtcctaggcgcagaaatcactggcgtagatttgcgcgaaccactggacgattcaacatggaacgagatactagacgccttccacacatatcaagtcatctatttccctggtcaagcaataacgaatgagcagcacattgcattctctaggagattcggaccggtcgatccggtaccgcacttaaaaagtattgaagggtatcctgaagttcagatgataaggagagaagccaatgaatcattgcgagtgatcggcgatgactggcacactgactccaccttcctagatgctccccctgccgctgttgtaatgagagcaattgatgtacccgaacacggaggggatacagggtttttgtcgatgtatacagcgtgggaaaccttgtcccccacgatgcaagcaaccatcgaggggttaaatgtagttcattccgcaactcgtgtattcggttccttgtatcaagctcaaaaccttcggtggtctaacacttcgctgaaagtcatggatgtcgatgcgggagacagagaaactgtacaccccttagttgtaacgcatccaggatctggacggaaaggcctgtatgtcaatcaagtgtattgtcagaggatagagggcatgactgatgctgaatccaaacccctcttgcaatttttatatgaacatgcgacccgtttcgattttacatgcagggtgcgctggaagaaggaccaagtgctggtgtgggataatttatgcacaatgcacagagcggtcccagattatgcaggcaagtttcggtacttaactagaactaccgttggaggggtgaggccagcgaggtga

SEQ ID NO: 94 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNWRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 95 - DNA, 888 bp, synthetic construct
  source 1..888: mol_type = other DNA
  CDS 1..888: protein_id = 94; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctctgtctccgctgtctcagcgttttgaacgtattgctgttcagccgctgaccggtgttctgggtgctgaaattactggcgtagatctgcgtgaaccgctggatgacagcacctggaacgaaatcctggatgcctttcatacctaccaggtaatttactttccgggtcaggctattaccaacgaacagcatattgcgttcagccgccgctttggtccggtggatccagtaccgcacctgaaatccatcgagggctaccctgaagtccagatgatccgtcgtgaagctaacgaaagcctgcgtgttatcggtgatgactggcataccgactctactttcctggatgcaccgccggcagctgtagttatgcgcgctatcgacgttccggagcacggtggtgatacgggctttctgtctatgtataccgcttgggaaaccctgagcccgactatgcaggcgaccatcgaaggcctgaacgttgttcacagcgcaacccgtgtattcggttccctgtatcaggcacagaactggcgttggagcaacacttctctgaaagttatggatgttgacgcgggcgatcgtgaaaccgtacacccactggtagttacccacccgggctctggtcgtaaaggtctgtacgtcaaccaggtttattgccagcgtatcgaaggtatgacggatgccgaatccaaaccgctgctgcaatttctgtacgagcacgcaacccgtttcgatttcacttgccgcgttcgttggaaaaaagatcaggtcctggtttgggataacctgtgcacgatgcatcgtgcggttccagattacgctggcaaattccgttatctgactcgtacgaccgtgggtggtgttcgtccggcacgttaa

SEQ ID NO: 96 - DNA, 888 bp, synthetic construct
  source 1..888: mol_type = other DNA
  CDS 1..888: protein_id = 94; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctcttagtccactctcgcagagatttgaaagaattgctgttcaaccactcaccggggttctcggagcggagattaccggcgttgatttgcgggaaccactcgatgactccacctggaacgagatcctcgacgcgttccacacctaccaagtcatttacttccccggacaggcgattaccaacgagcaacatatcgcgttctcacgccgtttcggcccagtcgaccccgttccgcacttgaagtcaatagaggggtatcctgaagtgcagatgatcaggagggaggctaacgagtcacttcgggtgatcggggatgattggcacacagatagtaccttcctcgatgccccacctgccgccgttgttatgcgcgcgatagatgtcccggagcacggcggggacaccggcttcctgtcaatgtatactgcttgggagacattgtcaccgacgatgcaagccacaattgaaggacttaacgttgtgcactccgctacacgtgtgttcggctcgttgtaccaggctcagaactggagatggtccaacacctccctcaaggttatggacgttgatgctggcgaccgtgagacggtccacccgctcgtcgttacccacccaggctcgggtaggaaggggttgtacgttaatcaggtgtattgccaaaggattgagggcatgaccgatgccgaaagcaagcccctcctgcaattcctgtatgaacatgctactcgcttcgatttcacctgccgcgtgcgttggaagaaggaccaggttctcgtttgggacaatttgtgcacaatgcacagagccgtccccgattacgctgggaaattcaggtaccttacgcggacaactgtcggtggtgttcggcctgcccgctga

SEQ ID NO: 97 - DNA, 888 bp, synthetic construct
  source 1..888: mol_type = other DNA
  CDS 1..888: protein_id = 94; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcattgtctccactctcgcaacggttcgaaagaatagctgtgcagccgctcacaggcgttttgggcgccgaaattactggtgtcgatttaagggaaccccttgatgactcaacttggaatgaaatcctggacgcattccatacataccaagtcatatattttcctggccaggcaataacaaacgagcaacatatcgctttttcacgacgctttgggccagttgacccggtcccacacctaaaatctattgagggatatccggaagtgcagatgattagacgcgaggcaaacgagtccctacgggtgataggcgatgattggcacaccgacagcacctttcttgacgcccctcccgccgcggtggtgatgcgcgcgatagacgtgcccgaacatggaggagatacagggtttttaagtatgtatacagcgtgggagactctttcgcctactatgcaagccacgatagaaggactcaacgtggtacattccgcgactcgggtgttcggaagtttatatcaggcgcagaattggcgctggagtaacacatccttaaaggtgatggatgtcgacgcaggggaccgagaaacagttcatccccttgtggtgactcaccccggctcgggccggaaaggcctttatgtcaaccaagtgtattgccaacgcatcgaaggcatgaccgacgcggaaagcaaaccattattacaatttttgtatgagcacgcaactaggtttgacttcacttgccgggtcagatggaaaaaagatcaggttctggtatgggataacttatgtacaatgcacagagccgtcccagattatgcaggtaagttccgatatttgactagaacgactgtgggaggagttagacctgctcgctga

SEQ ID NO: 98 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPTPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 99 - DNA, 888 bp, synthetic construct
  source 1..888: mol_type = other DNA
  CDS 1..888: protein_id = 98; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcgctgtctccgctgtctcagcgttttgaacgtattgcggtccagcctctgactggtgtactgggcgcggaaatcactggtgttgatctgcgtgaaccgctggatgattctacttggaacgaaatcctggatgctttccatacctaccaggtgatctatttcccgggtcaagcaatcaccaacgaacagcacatcgcattctctcgtcgtttcggtccggtagatccaactccgcacctgaaatccatcgaaggttatccggaagtgcaaatgatccgtcgtgaggcgaacgaatctctgcgtgtaatcggtgacgattggcacaccgattccaccttcctggatgcaccacctgctgccgtggttatgcgtgctattgacgtaccggaacatggtggtgacaccggtttcctgtctatgtacacggcctgggaaaccctgtctccgaccatgcaagccaccatcgaaggtctgaacgtcgtgcactccgcaacccgtgtattcggtagcctgtaccaggcgcagaatcgtcgctggtctaacacttctctgaaagtcatggacgttgatgctggcgatcgtgaaaccgtacacccgctggtagtaactcatccgggttccggtcgtaaaggtctgtacgtcaaccaggtatactgtcagcgcatcgaaggcatgactgatgcagaatccaaacctctgctgcagttcctgtacgaacacgctactcgctttgacttcacgtgccgtgttcgttggaaaaaagaccaggtgctggtctgggataacctgtgtaccatgcaccgtgcggtaccggattatgcgggtaaattccgttatctgactcgtactactgttggcggcgtgcgtcctgcgcgttaa

SEQ ID NO: 100 - DNA, 888 bp, synthetic construct
  source 1..888: mol_type = other DNA
  CDS 1..888: protein_id = 98; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcactctctcccctgtcacaacgttttgaacgcatcgccgtccaaccgttgactggtgtcctcggggcagagatcaccggcgtcgaccttcgcgagcctctcgatgattccacatggaacgagattctcgatgctttccatacatatcaggtcatctacttcccgggccaggcgatcaccaacgaacaacacatcgcgttctccaggaggttcggccccgtcgatccaacgcctcacctgaagagtatcgaggggtacccagaagttcaaatgattcggcgtgaagccaacgagagcttgcgcgtgatcggcgacgactggcacacagactccacctttctggacgccccgccggcagccgttgttatgagggcgattgacgttcctgaacacggaggagacaccggttttctgtcaatgtacaccgcctgggagacgctttcgcctactatgcaggccaccatcgagggcctgaatgttgtccattcagccacccgtgtgttcggctcgctttatcaggctcagaataggcggtggtcgaacacctccttgaaggtcatggatgtggacgccggcgatagagagactgtccatcccctcgtcgttactcaccccggcagcggtaggaagggtttgtatgtcaatcaggtttactgccagaggattgagggaatgactgatgcagagtccaagccgttgctgcagttcttgtacgaacacgcaacccgtttcgattttacctgtcgcgtcaggtggaagaaggaccaggttttggtgtgggacaacctctgcactatgcaccgggccgtgcctgattatgccggaaagtttcggtacctgactagaacgaccgtgggtggggttaggccagcgaggtga

SEQ ID NO: 101 - DNA, 888 bp, synthetic construct
  source 1..888: mol_type = other DNA
  CDS 1..888: protein_id = 98; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcacttagcccactatctcaacgcttcgaacgaatcgcagtccaacccttaacgggtgtgttgggcgcagagatcacgggtgtggatctgagagaaccacttgacgattccacctggaatgaaatccttgatgcgttccatacatatcaagttatatactttcccgggcaggcgattaccaacgagcagcacatcgcgttttcgcgacgctttggaccagtcgatcctacgccccatttaaaatcaatagaagggtatcccgaagtacaaatgattaggcgcgaagcgaacgagtcattaagggtaatcggcgacgactggcatactgatagtacgtttttggatgcaccccctgccgcggttgtcatgagagctatagacgttccggagcatggcggtgataccggatttctctctatgtacacagcctgggaaacactgtctcctaccatgcaagctactatagaaggtctaaacgtcgttcactcagcgacaagagtatttggttcactatatcaagcacaaaataggcgttggagcaatacctctttgaaggtcatggacgtggacgcgggcgatcgggagactgttcaccctttggtcgtaacgcacccaggatcaggtcgtaagggtctgtacgtcaatcaagtttactgccagcgcattgagggcatgacggatgctgaatctaaacctttactgcaattcttgtatgagcacgcgacgcgatttgacttcacctgtcgcgttaggtggaaaaaagatcaggtattagtctgggataacctgtgtacgatgcatagagctgtaccggactacgcaggaaagttcagatacctaactaggacaactgtcggcggggtgaggcctgcccgatga

SEQ ID NO: 102 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHADSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 103 - DNA, 888 bp, synthetic construct
  source 1..888: mol_type = other DNA
  CDS 1..888: protein_id = 102; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHADSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgagcccactgtctcagcgtttcgaacgtatcgctgtgcagccgctgaccggtgtactgggcgcggaaatcaccggtgttgacctgcgtgaaccgctggacgatagcacctggaacgagatcctggacgcctttcacacctaccaggttatctattttccaggccaggcgatcaccaatgaacagcacatcgcattctctcgccgttttggcccagttgatccagtgccgcacctgaaatctatcgaaggctatccggaggttcaaatgattcgtcgtgaagcgaatgaaagcctgcgtgtcatcggcgatgattggcatgccgactccaccttcctggacgctccgccagcggcggtggttatgcgcgcaatcgacgttccggaacatggcggcgacactggtttcctgagcatgtacaccgcgtgggaaaccctgtctccgactatgcaggccactatcgaaggtctgaacgtggtgcactctgcgacccgtgtattcggctccctgtaccaggctcagaaccgtcgctggagcaataccagcctgaaagtgatggacgttgatgcgggcgaccgtgaaactgttcatccgctggttgtgacccacccgggctctggtcgcaaaggtctgtacgtgaaccaggtttattgccagcgcatcgaaggcatgactgatgcggagagcaaaccgctgctgcagttcctgtatgaacacgcgactcgtttcgactttacctgtcgtgtgcgctggaagaaagaccaagttctggtatgggacaacctgtgcaccatgcaccgcgctgttccggactatgcaggtaagttccgttatctgactcgtactaccgtgggtggtgtccgcccggcgcgttaa

SEQ ID NO: 104 - DNA, 888 bp, synthetic construct
  source 1..888: mol_type = other DNA
  CDS 1..888: protein_id = 102; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHADSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcggctctgtctccactttctcaaagattcgagaggatcgcagtccagccacttaccggtgtgttgggggctgagattaccggcgtggatctcagggaaccactggatgactccacctggaatgagatcctcgatgccttccatacctaccaggtgatttattttcctggccaggcgatcactaacgagcagcacatagcatttagccgtaggttcggcccagtggacccagtcccacatctcaagagcatagagggttatccagaagtccagatgatcaggcgcgaagcgaacgagtccttgcgcgtgattggagatgactggcacgccgactccacatttctggatgcgccaccagctgccgttgtcatgagagccatcgacgtgcctgaacacggaggggacaccggattcctcagcatgtacactgcttgggaaaccttgtctccgaccatgcaggcgactattgagggcttgaacgttgttcattcagctacccgggtttttgggagcctgtaccaggctcaaaaccggaggtggtcaaatacgtctttgaaggtcatggatgtggatgctggagatagagagaccgtccatccccttgtcgtcacacatcccggtagcggtcggaagggcttgtacgtgaaccaggtgtactgtcagcggattgaggggatgaccgacgctgagtccaagccactcctgcaattcctgtatgagcatgcgacccgcttcgactttacctgccgggtgcggtggaaaaaggaccaggtgctggtctgggacaatttgtgcactatgcatcgcgctgtccccgattatgcaggtaagttcaggtacttgaccaggacaacggtcggtggcgtgagacctgctaggtga

SEQ ID NO: 105 - DNA, 888 bp, synthetic construct
  source 1..888: mol_type = other DNA
  CDS 1..888: protein_id = 102; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHADSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactatcgcccctcagtcagaggttcgaaagaatagcagtccaaccactaactggagtgcttggagcggagattacgggagtagacttgcgtgaaccacttgatgacagtacttggaacgaaatcttagatgcgtttcacacataccaagtaatatattttcccggccaggcgattaccaatgaacagcacatagctttttcgcgccgttttggcccagtagaccccgttccgcatcttaagtcgatcgagggttacccggaggttcaaatgattcgccgggaagcgaatgaatctttacgtgtaattggtgacgactggcatgcagattctacctttctagacgcgcccccagcggccgtggtcatgcgggccattgacgtgccagaacatggcggcgatacaggctttctttctatgtacaccgcctgggaaactttgtcccccaccatgcaggctactatagagggtcttaacgttgtacattctgccacgagagtgtttgggtctctgtatcaagctcaaaatcgccgttggtctaatacgagtcttaaagtgatggacgtcgacgctggggatcgtgaaacggtacatccgctcgtggttacacatcctggatcaggcagaaaagggttatatgttaaccaagtgtattgtcagcggatagagggaatgacggacgccgaaagcaagcccctgctacaatttttatatgagcacgcgacaagatttgattttacttgtcgtgtcaggtggaagaaggatcaggtgcttgtatgggataatctctgtacaatgcaccgggctgtgcctgattatgcgggaaagttcaggtatctgacgaggactactgtagggggagtacgccccgcacgttga

SEQ ID NO: 106 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHSDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 107 - DNA, 888 bp, synthetic construct
  source 1..888: mol_type = other DNA
  CDS 1..888: protein_id = 106; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctctgtccccgctgtcccaacgttttgaacgtattgcagtccaaccactgaccggtgttctgggtgctgaaatcactggcgtagatctgcgtgaaccactggatgactctacttggaacgaaattctggacgcgttccacacgtaccaggttatctacttcccgggccaggcaatcactaacgagcagcatatcgcattctctcgtcgtttcggtccggttgatccggtaccgcacctgaaatctatcgaaggctacccggaagtacagatgatccgtcgtgaggctaacgaatccctgcgcgtcatcggcgatgattggcacagcgattctaccttcctggacgctccgccggctgcagttgttatgcgtgccattgacgttccggaacacggcggtgacaccggtttcctgtctatgtataccgcatgggaaaccctgagcccgactatgcaggcgaccattgaaggcctgaacgtggttcacagcgcgacccgtgtgtttggtagcctgtaccaggctcagaaccgtcgttggagcaatacctctctgaaggtgatggatgtggacgcgggcgaccgtgaaaccgtccatccgctggtggttactcatccgggttccggccgtaaaggcctgtacgtaaaccaggtttactgccagcgcattgagggcatgaccgacgcggaatctaagccgctgctgcagttcctgtatgaacatgcaactcgtttcgatttcacctgccgtgtacgttggaaaaaagaccaagtactggtatgggataatctgtgcactatgcaccgcgctgttccagactacgctggcaaattccgttacctgacccgtactactgttggtggtgtgcgtccggctcgttaa

SEQ ID NO: 108 - DNA, 888 bp, synthetic construct
  source 1..888: mol_type = other DNA
  CDS 1..888: protein_id = 106; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcgctttcaccgctctcccaaagatttgaaaggatagctgtgcaacccctcaccggtgtccttggagctgagattaccggtgtggacctgagggaaccactcgatgactctacttggaacgaaatcttggatgcgttccacacttaccaggtgatttacttccctggtcaggcaatcaccaacgaacagcatattgccttcagcaggcgttttggccctgttgacccagtgccccatttgaagtccatcgagggttaccccgaagtgcaaatgatacgtcgcgaggcgaacgaatcattgcgggtcatcggcgatgactggcactcggactcaacttttctggatgcccccccagcggcggtcgtgatgagagccatcgatgttccagagcatgggggagacaccgggttcctcagcatgtatacagcctgggagactctcagccccaccatgcaggccacaatcgaggggctcaatgttgtgcactcagccacaagagtctttggctccctctaccaggcccagaaccgccgctggagcaacacctctctgaaggtgatggatgttgacgctggcgaccgggagaccgtccaccctcttgttgttacccatcctggaagtggaaggaaagggctgtatgtgaatcaggtctattgccagcgcattgagggcatgactgacgctgagtccaaaccgcttttgcaattcctctacgagcacgcaaccagatttgacttcacctgcagggtgaggtggaagaaagaccaggttctcgtttgggacaacctgtgcaccatgcatagggccgtgccagattacgcaggcaagttccgctacctgacaaggacgaccgtgggaggcgtccgtccggcgaggtga

SEQ ID NO: 109 - DNA, 888 bp, synthetic construct
  source 1..888: mol_type = other DNA
  CDS 1..888: protein_id = 106; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcgttatctcctctgagccaaagattcgaaagaattgcagttcaacccttgacgggtgtcttaggggccgagatcacgggcgtagatctccgcgagcctcttgacgatagcacctggaatgaaatattggatgctttccatacatatcaggttatatatttcccagggcaagctattacgaatgaacaacatatcgcttttagccgtcgcttcgggccagtggatcctgtaccacacttgaaatcaattgaaggatatccggaagttcagatgattaggcgtgaagctaacgagtcgttaagagttattggtgacgattggcactctgacagcacattcttagatgcaccccccgccgccgttgtaatgcgcgccatcgatgtccctgaacatgggggagacacgggtttcttatcaatgtacacagcgtgggagaccttgtcgcccactatgcaagccactatcgaaggtctcaatgtagttcatagcgctactagggtcttcggaagcctctatcaagcgcagaatcggagatggagtaatacatcacttaaagtaatggacgtagacgcaggggacagagaaaccgtacatcccctcgtagtcacccacccgggaagcggcaggaagggattatacgttaatcaagtctactgtcagaggattgagggcatgacagacgccgaatctaaaccgctccttcaatttctgtacgaacacgcaacgagatttgactttacatgtcgtgtaagatggaagaaagaccaagttctggtatgggacaatctctgtacgatgcaccgcgccgttccggactatgccggaaaattccgttatctgacaaggacgacggtggggggcgtgagacccgcacgttga

SEQ ID NO: 110 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHMDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 111 - DNA, 888 bp, synthetic construct
  source 1..888: mol_type = other DNA
  CDS 1..888: protein_id = 110; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHMDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcgctgtctccactgtctcagcgttttgaacgtattgcagttcagccgctgactggcgtactgggtgctgaaattaccggcgttgacctgcgtgaacctctggacgactctacttggaacgaaattctggacgcgttccacacctaccaggttatctattttccgggtcaggctatcaccaacgagcagcacattgcttttagccgtcgtttcggccctgtcgatccggttccgcacctgaaaagcattgaaggctatccagaagttcagatgatccgtcgtgaagctaatgaaagcctgcgcgtcattggtgacgactggcacatggatagcacgttcctggatgcgccacctgcggcagttgttatgcgtgcgattgacgttccggagcacggtggcgacactggtttcctgagcatgtacactgcctgggaaacgctgtctccgaccatgcaggctaccatcgaaggcctgaatgtcgttcactccgccacccgtgttttcggttccctgtatcaggcgcagaaccgtcgttggtctaacacttctctgaaagttatggatgttgacgccggtgaccgtgaaaccgtacacccgctggtggttacccatccgggttctggtcgtaaaggtctgtacgtgaaccaggtttactgccagcgtatcgaaggtatgactgacgcggaatccaagccgctgctgcagttcctgtatgaacacgcaacgcgtttcgacttcacttgtcgtgtgcgctggaaaaaagatcaggttctggtgtgggacaacctgtgtaccatgcaccgtgcagtgccggactatgctggtaaattccgttacctgactcgtaccaccgttggtggtgttcgcccagctcgttaa

SEQ ID NO: 112 - DNA, 888 bp, synthetic construct
  source 1..888: mol_type = other DNA
  CDS 1..888: protein_id = 110; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHMDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgccttgtcgcctttgtcccagagatttgaaagaattgcagtccagcccctgactggcgttctgggcgctgagatcactggggtggatctgcgcgagccactggatgactctacgtggaacgaaattttggatgccttccatacctaccaggtcatctacttccccggccaagccatcacgaatgagcagcacatcgcgttttcacgtagattcgggccggtggatcctgtgcctcacctgaagtccattgaggggtaccccgaggtgcaaatgattagaagagaggccaacgaatcccttcgcgtcatcggtgatgactggcacatggactccacattccttgatgcacccccagccgcggtggtcatgagggccattgacgtgccggagcatgggggagacaccggttttctgtctatgtataccgcctgggagaccctgtctcccaccatgcaggctaccatagagggccttaatgtggtccactcagccaccagggtctttgggagtttgtatcaggcccagaacaggcggtggtctaacactagtctcaaagtcatggatgtggacgcgggagaccgcgaaacagttcacccactggtggttacacacccgggcagtggaagaaagggcctgtatgtgaatcaagtttactgccagcgcattgaagggatgaccgacgcggagtcgaagccattgctgcaattcttgtatgagcacgcgacacggttcgactttacttgcagggtgagatggaagaaggaccaggtgctggtctgggataatctgtgcaccatgcaccgtgctgtcccagactatgccggcaagttccggtaccttaccaggactaccgttggaggagtgaggcctgctaggtga

SEQ ID NO: 113 - DNA, 888 bp, synthetic construct
  source 1..888: mol_type = other DNA
  CDS 1..888: protein_id = 110; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHMDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctctaagtccactgtcacaacgctttgaacgcatcgcggtgcagcccctcactggtgtattaggagcggaaataaccggagtagacttacgagaacccttggatgattctacatggaatgagatcctcgatgcattccatacttaccaggtgatatacttccccggacaggccataacaaacgaacagcatatcgccttctccaggcgctttggacccgtcgatccagtcccgcatcttaagagcatcgaaggataccctgaagtgcaaatgattcgccgcgaggcaaacgaatccctgagagtaattggagacgattggcatactgactctaccttccttgacgctccccccgcagctgttgtaatgagagcgatagacgtccccgaacatgggggcgacactggctttttgtcaatgtacacagcgtgggaaactttatcacctactatgcaagcaactattgaaggtctcaacgtagtccactctgccacaagggtatttggatcactataccaagcacaaaatcgccgctggagcaacacctcgcttaaggtgatggatgttgatgccggagatcgtgaaacggtacatcccctggttgtgactcacccagggtcgggcaggaaaggcctttacgtcaatcaggtttactgtcaacggatagaaggaatgacggacgcagagtcgaaaccacttttgcaattcttatatgagcatgccaccaggtatgatttcacatgccgcgttcgctggaagaaagatcaggtgttggtttgggacaacctctgcacaatgcatagggccgtaccggattatgcgggaaaatttcgttacctaacgcgaacaacggtggggggagtacgccctgcgagatga

SEQ ID NO: 114 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRYDFTCRVRWKKDQVLVWDNLCTMHRAVPDYAGKFRYLTRTTVGGVRPAR

SEQ ID NO: 115 - DNA, 888 bp, synthetic construct
  source 1..888: mol_type = other DNA
  CDS 1..888: protein_id = 114; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRYDFTCRVRWKKDQVLVWDNLCTMHRAVPDY AGKFRYLTRTTVGGVRPAR
atgcacgcagctctgtccccgctgtctcagcgttttgaacgtattgctgttcaaccgctgaccggcgttctgggcgcagaaattaccggcgtagacctgcgtgaaccgctggatgatagcacttggaacgagatcctggatgcttttcacacctaccaggtgatctatttcccgggccaggctattaccaacgagcagcacatcgcgttctcccgtcgtttcggtccggtagacccggtccctcacctgaaatctatcgagggttatccggaagttcagatgatccgtcgtgaagcaaacgaaagcctgcgcgtcatcggcgatgactggcataccgattctacttttctggatgcaccgccagcagcggtagttatgcgtgcgatcgatgtcccagaacacggtggtgataccggcttcctgtctatgtacaccgcttgggagactctgtctcctactatgcaggccaccattgaaggcctgaacgtggtgcactccgctactcgcgtttttggctccctgtaccaggcacagaaccgtcgttggagcaacacctccctgaaagtgatggacgttgacgcgggtgatcgtgaaaccgttcatccgctggtcgtaactcacccaggttctggtcgtaaaggcctgtacgtgaaccaggtgtattgccagcgtatcgaaggcatgaccgatgcagaatctaagccgctgctgcagttcctgtacgaacacgcgacccgttacgacttcacttgtcgtgtgcgttggaagaaagatcaggtcctggtttgggacaacctgtgcaccatgcaccgtgcggtaccggactatgcgggtaaattccgttacctgacccgtactaccgtcggtggtgttcgtccggcacgctaa

SEQ ID NO: 116 - DNA, 888 bp, synthetic construct
  source 1..888: mol_type = other DNA
  CDS 1..888: protein_id = 114; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRYDFTCRVRWKKDQVLVWDNLCTMHRAVPDY AGKFRYLTRTTVGGVRPAR
atgcatgccgctctcagtcctttgagccaacgcttcgaaaggattgcggtccaaccgctgactggggttctgggcgctgagatcacgggagttgatctccgtgagcccctcgatgactccacatggaatgaaattcttgacgcgtttcacacgtaccaagtgatttatttccccggccaagcgatcacaaatgaacagcacatcgctttctcaaggcggttcgggcctgttgacccagtgccccacttgaagtcgattgagggctacccagaagttcagatgatcaggcgggaagcgaacgagtccctccgcgtgatcggcgatgattggcataccgacagcacattcctcgatgcgcccccagcagctgtcgtgatgcgggccatcgacgtgccagaacatggcggtgacaccgggtttctgtctatgtacacagcatgggagactcttagccctaccatgcaggcgactatcgagggcctgaacgtggttcactctgccactagggtcttcggcagcctgtatcaggcgcaaaaccgtcggtggagcaacacgagtcttaaggtcatggacgtcgacgccggggaccgcgagacggttcacccccttgttgtgacacatccaggctcaggcaggaagggcttgtatgtgaaccaggtctactgccaaaggatcgaaggaatgacagatgcggagtcgaagccactcctccaatttctgtacgaacatgccaccaggtacgatttcacctgcagggtccgctggaagaaggaccaggtcctggtctgggataacctctgcaccatgcatcgggccgttcccgactatgcaggcaaatttcgctatctcacccgcactactgtcggcggcgtccgcccggctagatga

SEQ ID NO: 117 - DNA, 888 bp, synthetic construct
  source 1..888: mol_type = other DNA
  CDS 1..888: protein_id = 114; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRYDFTCRVRWKKDQVLVWDNLCTMHRAVPDY AGKFRYLTRTTVGGVRPAR
atgcatgcggccctgtcaccgctttcacaaagattcgaaagaatcgcagtccagccgttaactggagtcctaggcgcggaaattactggcgtggacttgcgtgagcccttggacgactcgacctggaatgaaattttagacgcgtttcacacatatcaggtaatctacttccctggtcaagcaattacgaatgagcaacacattgctttttctcgccggtttggtcctgttgatcctgtaccccatctaaaatcaattgaggggtacccggaagtgcagatgatccggagagaagcgaacgaatcgttgcgagtaattggagatgactggcacacggactccacgttcctagatgccccaccagctgcggtagttatgcgagcaattgatgtaccagagcacggcggggacacgggtttcctttcgatgtatacagcttgggaaacgttgagtcctacgatgcaagcaacgatcgaaggattaaacgtcgtgcattcagccacacgcgtcttcggatcgctgtaccaggcccaaaacagacgttggtcaaacacttccctgaaggttatggacgtagatgcgggtgacagggagactgtgcacccattagtagtaacccacccagggtcggggcgcaaaggactgtatgttaatcaagtttattgccagagaattgaagggatgaccgacgccgaatcgaaacctctcttgcagttcttgtacgagcacgctacccgctacgacttcacgtgccgtgttcgctggaagaaggatcaggtattggtttgggacaatttgtgtactatgcatcgggcagtcccggattacgcgggcaaatttcgatacctaactagaacgacagttggtggggttcggccagcgagatga

SEQ ID NO: 118 - protein, 295 aa, synthetic construct
MHAALSPLSQRFERIAVQPLTGVLGAEITGVDLREPLDDSTWNEILDAFHTYQVIYFPGQAITNEQHIAFSRRFGPIDPVPHLKSIEGYPEVQMIRREANESGGVIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 119 - DNA, 888 bp, synthetic construct
  source 1..888: mol_type = other DNA
  CDS 1..888: protein_id = 118; transl_table = 1; translation = MHAALSPLSQRFERIAVQPLTGVLGAEITGVDLREPLDDSTWNE ILDAFHTYQVIYFPGQAITNEQHIAFSRRFGPIDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgtccccgctgagccaacgtttcgaacgtattgcggtacagccgctgaccggtgttctgggtgctgaaatcactggtgtggacctgcgtgaaccgctggacgactctacttggaacgaaatcctggatgccttccacacttatcaggtgatctactttccgggtcaggctatcaccaacgaacagcatatcgctttttctcgtcgtttcggtccgatcgatccggtgccgcacctgaaatctattgagggttatcctgaagtacagatgattcgtcgtgaagctaacgaatccggcggcgttattggtgatgattggcacacggacagcaccttcctggatgctccgccggccgctgttgttatgcgtgcaatcgatgtaccagaacacggcggtgacacgggcttcctgtccatgtacactgcgtgggaaacgctgagcccgaccatgcaggcaaccattgaaggcctgaacgttgtccactccgctacccgtgtattcggttctctgtaccaggcgcaaaaccgtcgttggtctaacacctccctgaaagtgatggacgtagatgcgggcgatcgtgaaactgtccacccgctggtagtaacccaccctggctctggtcgtaaaggtctgtacgtcaaccaggtttattgccaacgtattgaaggcatgaccgatgcggaatccaaaccgctgctgcagttcctgtacgaacacgcgactcgttttgactttacctgccgcgtccgttggaaaaaagatcaggttctggtttgggataacctgtgcactatgcatcgtgccgttccggattacgctggtaaatttcgctacctgactcgtaccactgtgggtggcgtacgtccggctcgttaa

SEQ ID NO: 120 - DNA, 888 bp, synthetic construct
  source 1..888: mol_type = other DNA
  CDS 1..888: protein_id = 118; transl_table = 1; translation = MHAALSPLSQRFERIAVQPLTGVLGAEITGVDLREPLDDSTWNE ILDAFHTYQVIYFPGQAITNEQHIAFSRRFGPIDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcgttgtccccccttagccagcggtttgagagaatagccgttcagcctttgactggggtgctcggagccgagatcaccggcgtcgacctccgcgagcctctggacgacagcacgtggaacgagatcctcgacgccttccacacataccaagttatctactttccggggcaggctattacaaacgagcaacacatcgctttttcgaggaggtttggccccatagatccagtcccacacctcaagagcatcgagggatacccagaggtgcagatgatccgtagggaagccaacgagtcgggtggagtcatcggcgacgactggcataccgattcgacgtttctcgatgcgcctcctgccgcagttgtcatgcgggcaatcgacgtcccggagcacggcggcgatacaggtttcttgagcatgtacaccgcctgggagactctgtcccctacgatgcaggccactatcgaaggcctgaacgtggtgcatagcgccaccagggtttttggcagcttgtaccaagcacagaacagaagatggtctaacacttcgctcaaggtcatggacgtggatgccggcgatagggagactgtgcacccgctcgtcgtcactcacccgggctcaggtaggaagggtctgtacgttaaccaggtgtactgccagcgcatcgaaggcatgacggacgctgagtcaaaaccccttctgcaattcctctacgaacatgccacaaggtttgacttcacatgtcgggttcgctggaagaaggatcaggttcttgtctgggacaatctgtgtacgatgcatagggccgtgccggattacgccggcaagtttcgctatctcacaaggactaccgtcggcggcgttaggcctgcccgctga

SEQ ID NO: 121 - DNA, 888 bp, synthetic construct
  source 1..888: mol_type = other DNA
  CDS 1..888: protein_id = 118; transl_table = 1; translation = MHAALSPLSQRFERIAVQPLTGVLGAEITGVDLREPLDDSTWNE ILDAFHTYQVIYFPGQAITNEQHIAFSRRFGPIDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactctcacctctctcccaacgttttgagaggattgctgtgcaaccgcttaccggcgtactcggtgctgaaattaccggcgttgatctcagggagcctcttgacgattctacatggaacgaaattctcgatgccttccatacttatcaggtcatctatttcccaggccaagctataaccaatgaacagcatattgctttcagccgtaggttcggtcctattgaccctgttcctcatctcaaatcaatagaggggtatcctgaagtccaaatgattagaagggaagccaacgaaagcggtggtgtgatcggtgatgattggcataccgatagcaccttcttggatgcccctcctgcagctgttgtaatgcgagcaatcgacgttcccgaacatggcggagacaccggcttcctttctatgtacacagcttgggaaactctttcaccaactatgcaagcaactatagaaggacttaacgttgttcattcagctactagggttttcggatcactctatcaagctcagaatcgtagatggagtaacactagccttaaagtcatggatgtagacgctggagacagggagactgttcatcctctcgttgttactcaccctgggagcggtagaaaagggctctatgttaaccaagtttattgccagagaattgagggaatgactgatgctgagtcaaaacctctcttacagtttttgtatgagcatgctacaaggtttgacttcacctgcagggttcgttggaagaaggatcaagttcttgtatgggacaacttgtgcaccatgcatcgagctgttcccgattacgcaggcaagtttcgttatttgacacgaacaactgtcggcggtgttaggcctgcaaggtga

SEQ ID NO: 122 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLGYIGDDWHTDSTFLDAPPAAVVMRAIDVPEHGGDTGFLSMYTAWETLSPTMQATIEGLNVVHSATRVFGSLYQAQNRRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 123 - DNA, 888 bp, synthetic construct
  source 1..888: mol_type = other DNA
  CDS 1..888: protein_id = 122; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LGYIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagctctgtccccgctgagccagcgttttgaacgtattgctgtgcaaccgctgaccggcgtactgggtgctgaaattactggtgttgatctgcgtgaaccgctggatgactctacctggaacgagatcctggacgcgttccacacctatcaggttatctatttcccgggccaggcaattaccaacgaacagcacatcgcattttctcgtcgtttcggtccggttgatccggttccacacctgaaatctatcgagggctacccggaggtccagatgatccgccgtgaagctaacgagagcctgggctacatcggtgatgactggcatacggattctacctttctggatgcaccgccggctgctgttgttatgcgtgccatcgatgtccctgagcacggtggcgacactggtttcctgtctatgtacaccgcgtgggaaactctgagcccgacgatgcaggctactattgaaggtctgaacgtagtacacagcgctacccgtgtgttcggttctctgtatcaggctcagaaccgtcgttggtccaacacctccctgaaagtcatggacgttgacgccggtgaccgtgaaaccgttcatccgctggtagtcacccacccgggttctggtcgtaaaggcctgtatgttaaccaggtgtactgccagcgtatcgagggcatgaccgacgctgaaagcaagccgctgctgcagttcctgtatgagcacgcaacccgttttgactttacctgccgtgttcgctggaaaaaggatcaggtactggtgtgggataacctgtgtaccatgcaccgtgctgttcctgactacgcgggcaaattccgttacctgacccgtactaccgttggtggtgttcgtccagctcgttaa

SEQ ID NO: 124 - DNA, 888 bp, synthetic construct
  source 1..888: mol_type = other DNA
  CDS 1..888: protein_id = 122; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LGYIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagccctgagccctctttcccagcgctttgaaagaattgccgtgcagccgctgaccggcgtcctgggggccgagatcacaggcgttgacctccgcgagcccttggatgacagcacgtggaacgaaattcttgatgcattccatacctatcaggtcatctactttccagggcaagctattaccaacgagcagcacatcgcattctccagacgcttcggcccagtcgatccagtccctcaccttaagagcatagaagggtaccctgaggtccaaatgattaggagagaggccaacgaaagtcttggctacataggcgatgactggcacacggacagtacgttccttgacgctccaccagctgcggtcgtcatgagggcgatcgacgttcccgagcacgggggtgataccgggttcctgtccatgtacactgcatgggagacactttctcccaccatgcaggcgacgatcgaggggctgaacgttgtccattccgctactcgcgttttcggctctctctaccaggcacaaaaccggcgctggtccaacacaagcctcaaggtgatggacgtggatgctggcgaccgggaaacggttcaccccttggttgttactcacccggggtctggcagaaagggtctctacgtgaaccaggtgtactgccaaagaattgagggtatgactgatgcagagtcgaagcccttgctgcaattcctgtacgaacacgccaccagattcgatttcacctgtcgggtgagatggaagaaagaccaagtgctggtgtgggacaacctctgcacgatgcatcgcgcggttccagactacgcaggcaagttccgctaccttaccagaactaccgtcgggggtgtgaggccggcccgctga

SEQ ID NO: 125 - DNA, 888 bp, synthetic construct
  source 1..888: mol_type = other DNA
  CDS 1..888: protein_id = 122; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LGYIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggcattatccccgcttagccagaggtttgaaagaatcgcagtgcagcccttaactggggttttaggcgcggagataaccggagtggacctgcgtgaaccacttgacgattccacatggaatgagattcttgatgcattccacacataccaagtaatttatttcccaggacaggccataacgaatgagcaacatatcgcgttttcacgcagatttgggcccgttgaccctgtccctcacttaaaatctattgaggggtaccctgaggtacaaatgattcgtcgtgaagctaacgaatcgctcggctatataggtgatgattggcacacggattctacttttctggatgctccgccggcggctgtagttatgagggcaatcgacgtcccagaacacggaggcgacacagggtttttatccatgtatacggcatgggaaacgttatctcctactatgcaggccacaatcgaagggttgaacgttgtccattcagcgactcgtgtttttggatcactgtatcaagctcagaatcgccgttggtcaaatacttccttaaaagtcatggacgttgatgccggagatcgggagaccgttcacccactagttgtaacccatccagggagcggcaggaaaggattgtatgtaaatcaggtatattgtcaacgcatagagggtatgacagacgcggaatccaagcctctgcttcaatttttgtatgaacatgccacacgttttgactttacgtgcagagtgcgctggaaaaaggatcaggtcttagtgtgggataatctctgcactatgcaccgtgcagtcccagactatgcagggaaatttagatatttgaccaggacaactgtcgggggagtgcggccggctaggtga

SEQ ID NO: 126 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHSDSTFLDAPPAAVVMRAIDVPEHGGDTGFLSMYTAWETLSPTMQATIEGLNVVHSATRVFGSLYQAQNKRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 127 - DNA, 888 bp, synthetic construct
  source 1..888: mol_type = other DNA
  CDS 1..888: protein_id = 126; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNKRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctctgtctccgctgtctcaacgttttgaacgtatcgcagttcagccgctgaccggtgttctgggcgctgaaattactggtgtggatctgcgcgaacctctggacgattctacctggaacgaaatcctggacgcttttcatacctatcaggttatctacttcccgggtcaggcaatcaccaacgaacagcacatcgctttctctcgtcgtttcggtccggttgacccggtgccacacctgaaatccatcgaaggttacccagaagtacagatgatccgtcgtgaagctaacgaatctggtggtgtaattggcgacgattggcattctgatagcactttcctggatgctccgccggcggcagttgttatgcgtgctatcgatgttccggaacatggcggcgacactggcttcctgtccatgtacactgcatgggaaaccctgtctcctactatgcaggcgaccatcgaaggtctgaacgtggtccactctgccacccgtgttttcggttctctgtatcaggctcagaacaaacgttggagcaacacttccctgaaagtgatggacgttgacgcgggcgatcgtgaaactgtgcacccgctggttgttacccaccctggttctggtcgcaaaggtctgtacgttaaccaggtttactgccagcgtatcgaaggtatgaccgacgcagagtctaaaccgctgctgcaattcctgtacgaacacgcgacgcgtttcgactttacctgccgcgtacgttggaaaaaagatcaggttctggtatgggacaacctgtgcaccatgcaccgtgcggttccggattacgcaggtaaattccgttacctgacccgcactactgtaggcggtgttcgcccggcacgttaa

SEQ ID NO: 128 - DNA, 888 bp, synthetic construct
  source 1..888: mol_type = other DNA
  CDS 1..888: protein_id = 126; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNKRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgccgctttgtcaccgctgtcccaaagatttgagaggattgcggtccagcccctcactggcgtgctcggtgctgagatcacaggtgtggacctgagagagccgttggacgattccacttggaacgagatcctggatgccttccatacctaccaagtcatatattttcctggacaagccataaccaacgagcagcacatcgccttcagccgcaggttcggccccgtggatcccgtccctcatctcaagtcgatcgaaggatacccagaggttcaaatgatcaggagggaggcaaacgagagtggcggtgtcatcggggacgattggcatagcgactctacgtttctggacgctccccctgcagctgtggtcatgcgcgcaatagacgttccagaacacggcggcgacactggcttcctctcgatgtatacggcctgggagactctctcaccgactatgcaggccacaatcgagggcctgaacgtcgtccactccgccacccgcgtcttcggctctctctatcaagcgcagaacaagaggtggtccaacaccagcctgaaagtgatggacgttgatgccggtgatcgtgagacggtccatccgcttgtggttacgcacccgggttccggccgcaagggactctacgtgaatcaggtgtactgtcaaaggattgagggcatgaccgatgctgagtcgaagccgctgttgcaatttctgtacgagcacgcaacaagattcgactttacctgccgggttcggtggaagaaggaccaagtcttggtgtgggacaatttgtgtaccatgcaccgggcagtcccggattatgccggtaagtttcgctatctgacaagaactaccgtcggcggagtccggccagctcgctga

SEQ ID NO: 129 - DNA, 888 bp, synthetic construct
  source 1..888: mol_type = other DNA
  CDS 1..888: protein_id = 126; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNKRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcgttgtcccctctgagtcaacgctttgaacgaattgcggttcagccattgactggggtgttaggagcggaaattacgggcgttgatcttcgtgaacctttggacgacagcacgtggaacgaaattcttgacgctttccacacgtaccaagtgatctactttcccgggcaggcaattactaatgagcagcatattgcattctcgcgtcgattcggacccgtcgatcccgttcctcatctcaaatctattgaaggctacccggaagtccagatgattagaagagaggcaaatgagtcgggaggtgtaatcggggacgactggcactctgactccacttttctggatgcaccccccgcagctgtagtgatgagggccatagatgtcccggagcatggaggcgatactggtttcctatcgatgtacactgcgtgggaaactcttagccccaccatgcaggcgactattgagggactaaatgtagtgcactcggccacgcgggtattcggatccttatatcaagcgcaaaataaacgatggagtaatacaagcctgaaagttatggatgtagatgctggagacagggagactgtgcaccctttagttgttactcatccaggaagtgggcggaagggcctttatgttaatcaagtgtattgtcaaagaatcgaaggaatgactgatgccgaatctaagccattgttgcagttcctgtatgaacacgccacgcgtttcgatttcacatgccgggtgagatggaagaaggaccaggtattagtgtgggataacctgtgcacgatgcatcgtgctgtccctgactatgcaggcaaatttcgctacctaactcgaactactgtaggaggggtaagacccgcgaggtga

SEQ ID NO: 130 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHSDSTFLDAPPAAVVMRAIDVPEHGGDTGFLSMYTAWETLSPTMQATIEGLNVVHSATRVFGSLYQAQNMRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 131 - DNA, 888 bp, synthetic construct
  source 1..888: mol_type = other DNA
  CDS 1..888: protein_id = 130; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgccctgtctcctctgtcccagcgtttcgaacgtattgctgtacagccgctgaccggtgttctgggcgcagaaattactggcgtcgatctgcgtgaaccgctggatgactccacctggaacgaaatcctggatgcattccatacttatcaggtcatctatttcccgggccaggcgattaccaacgaacagcacatcgccttttctcgccgttttggtccggttgatccggtgccgcacctgaaaagcatcgaaggttacccggaagtgcagatgatccgtcgtgaagcaaacgaatctggcggcgtgatcggtgacgattggcactctgattctaccttcctggatgctccgccggcggctgtagttatgcgtgctattgacgttccggaacatggtggtgacaccggtttcctgtccatgtacaccgcttgggaaacgctgtctccgaccatgcaggccaccatcgaaggtctgaacgtcgtgcacagcgcgactcgtgtgttcggcagcctgtatcaagcccagaacatgcgctggtctaacaccagcctgaaagtgatggacgttgacgcaggtgaccgtgaaaccgttcatccactggttgttacccatccgggtagcggtcgcaaaggcctgtacgtaaatcaagtctactgccagcgcatcgagggtatgaccgacgcagaatccaaaccgctgctgcagttcctgtacgaacacgcgacccgctttgatttcacctgccgtgtacgttggaaaaaggaccaagtactggtgtgggacaacctgtgcaccatgcaccgtgccgttccggactacgcaggtaaattccgttacctgactcgtactactgttggtggtgtacgtccggctcgttaa

SEQ ID NO: 132 - DNA, 888 bp, synthetic construct
  source 1..888: mol_type = other DNA
  CDS 1..888: protein_id = 130; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggccctctccccactgtcgcaaagatttgaaagaatcgctgtgcaacccctgacaggcgtgctgggagctgagattactggggttgacctccgggaacccctggatgactcaacctggaatgaaattttggatgcgttccacacctatcaggtcatctacttcccgggccaggcgattactaatgagcaacacatcgccttttcacgcaggtttggaccagtcgaccctgtccctcacctcaaaagcatcgagggatacccagaggttcaaatgatccggagggaggccaatgagtctggcggcgttatcggcgacgattggcactcggattcgactttcctcgatgcgcctccagccgccgtggtgatgagggctatcgacgtgcctgagcacgggggcgacaccggcttcctcagcatgtatactgcttgggagacgctgtcccctaccatgcaggctactatcgaaggcctgaacgttgtgcactccgcaacccgcgtttttgggagcctgtaccaggcccagaacatgaggtggagcaatacttcactgaaggttatggatgtcgacgctggggacagggagacagtgcatccactcgtcgtcacacatccggggtcgggccgcaaaggcctttacgttaaccaagtttactgccaacggattgagggcatgacggacgccgaatctaagcccctgcttcaattcttgtacgagcacgctaccaggtttgactttacatgccgcgtccgctggaaaaaggatcaggtgctcgtgtgggataacctttgtacaatgcaccgcgctgtgccagattacgcaggcaagttcaggtacctcacacgcaccacggtcggcggggtgaggccagccagatga

SEQ ID NO: 133 - DNA, 888 bp, synthetic construct
  source 1..888: mol_type = other DNA
  CDS 1..888: protein_id = 130; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcgcttagcccattgtcccagcgttttgagagaatagcagtacagcccttgactggagttctaggcgcagaaatcacaggggtcgatcttcgcgaaccgctagatgattccacatggaatgagatcctggacgcattccacacttatcaagtgatctattttccgggtcaggcaatcactaacgaacaacacattgctttttcgcgtagatttggcccggttgatcctgtgccgcatctcaaatctatagaaggatacccggaggttcagatgattagacgggaagccaacgagtctggtggggttattggggatgactggcattctgatagcacctttctggatgcacctccagcagcagttgtaatgcgggccatagacgtcccggaacacggaggagatactggtttcttgtcaatgtacaccgcatgggaaaccttgtccccaacgatgcaagcgacaattgaaggcctcaatgtcgttcactcagcaacaagggttttcggttcgctctaccaggcccaaaacatgagatggagtaacacttcgcttaaagtgatggacgtagacgcaggtgaccgggaaacagtacacccgctggtcgtcactcatcctgggtcaggtcgtaaaggactgtatgtaaaccaagtatattgccagcgcattgagggaatgacggatgcagagtcaaagcctctattacagtttttatacgagcacgcgacacgatttgacttcacttgtcgagtacgttggaaaaaggatcaagtactcgtttgggataacttatgcaccatgcatcgtgcggtccctgattatgccgggaagtttcgctatctaactagaaccactgtcggtggagttagacctgcgagatga

SEQ ID NO: 134 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHSDSTFLDAPPAAVVMRAIDVPEHGGDTGFLSMYTAWETLSPTMQATIEGLNVVHSATRVFGSLYQAQNWRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 135 - DNA, 888 bp, synthetic construct
  source 1..888: mol_type = other DNA
  CDS 1..888: protein_id = 134; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcactgtctccgctgtctcagcgttttgaacgcattgcggtacagccgctgactggcgtgctgggtgctgaaatcaccggtgttgatctgcgcgaacctctggacgattccacctggaacgaaatcctggacgcgttccacacctaccaagttatttacttcccgggccaggcaatcaccaatgaacagcacatcgctttctctcgtcgctttggtccggttgacccggttccgcacctgaaatccatcgaaggctacccggaggtgcagatgatccgtcgcgaagcgaacgaatctggtggtgttatcggcgatgactggcatagcgatagcacttttctggatgctcctccggctgcagtcgttatgcgcgctatcgacgtcccggaacacggtggtgatactggtttcctgagcatgtacaccgcgtgggagactctgtctcctaccatgcaggccaccatcgaaggcctgaacgtggttcattccgcgacccgtgtatttggttccctgtaccaggcgcagaactggcgttggtccaacacttccctgaaagttatggatgttgatgcaggcgaccgtgaaaccgtacatccgctggtggttacccacccgggttctggtcgtaaaggtctgtacgttaaccaggtgtattgccagcgcatcgaaggcatgactgacgctgaatccaaacctctgctgcagttcctgtatgaacacgcgacccgcttcgattttacctgccgtgtgcgttggaaaaaggaccaggtactggtgtgggacaacctgtgtaccatgcaccgtgctgttccggactatgcgggtaaattccgttacctgacccgtaccaccgttggcggtgtacgtccagctcgctaa

SEQ ID NO: 136 - DNA, 888 bp, synthetic construct
  source 1..888: mol_type = other DNA
  CDS 1..888: protein_id = 134; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcactttcacctctgagccagaggttcgagagaattgcggtgcaacccctcaccggcgtcttgggtgccgagattacgggcgtcgaccttagagaaccactggatgactctacatggaacgaaatcctggatgcctttcacacataccaggttatctattttccaggacaggcaatcactaacgaacagcacatagcgttctctagacgcttcggccccgtggacccagtgccgcatctgaagagcatagaaggttacccagaggtgcagatgattcgccgcgaggctaatgaaagtgggggcgtgattggggacgactggcattcggattccacgttcctggatgcgccaccggccgctgtcgtgatgagagcgatagacgtgccagaacacggtggcgacacggggttcctcagcatgtatacggcttgggagaccctcagccccaccatgcaggccacgatcgagggcttgaacgttgtccatagcgctacacgcgtgttcggcagtctctaccaggcgcagaactggaggtggtctaatacatcactcaaggtcatggatgtcgatgcgggggatcgcgaaacagtgcacccgctcgttgttacccatccgggcagcggtagaaagggcctgtatgtcaaccaagtttactgccaaagaatcgaaggaatgacggatgcggaatccaagcctttgctccagttcctgtacgaacatgctactcgctttgactttacatgccgcgtgcgctggaagaaggatcaggtgctcgtctgggataacctgtgtacgatgcatcgggctgtgccggattacgcgggcaagtttcgctacctgacgcggactacagtgggcggcgtgcgccctgcgcggtga

SEQ ID NO: 137 - DNA, 888 bp, synthetic construct
  source 1..888: mol_type = other DNA
  CDS 1..888: protein_id = 134; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcggcccttagtccattatctcaacggtttgaaaggattgcagtacagcctctcacgggcgtactaggggctgagataacaggtgtagatttgcgagagccactcgacgactccacatggaacgaaatactcgacgcatttcatacatatcaagtgatttatttccctggtcaagcgatcacaaacgaacagcatatagctttctccagacggttcggcccagtggacccagtgccccacttaaagtccattgaaggctatcccgaagtccagatgattagacgcgaggccaatgagtctgggggagtgatcggggatgactggcattctgactcgacctttctggacgccccgccagccgccgttgtgatgcgtgctatcgatgtccctgaacatggaggcgacaccgggtttctctctatgtatacggcctgggaaactctgagcccgacgatgcaagcgacaatagaagggctaaacgtcgtacactcagcgacaagggtttttgggtccctttaccaagcacaaaattggcgatggtctaataccagtttaaaagtaatggatgtcgatgcgggagatcgtgaaacagtccaccccctggtggtaactcatcctggcagtggtagaaaaggcctatatgtgaatcaggtctactgccaaagaatagaggggatgacagatgctgagagcaaaccactattacagttcttatacgaacacgccacgcgattcgattttacctgtcgcgttcgttggaaaaaagatcaggtgttagtttgggacaacctatgcactatgcaccgtgctgtaccggactatgccggaaagtttagatatcttacccgcacaactgtcggaggagttcgtcccgctcggtga

SEQ ID NO: 138 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESLRVIGDDWHTDSTFLDAPPAAVVMRAIDVPEHGGDTGFLSMYTAWETLSPTMQATIEGLNVVHSATRVFGSLYQAQNTRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 139 - DNA, 888 bp, synthetic construct
  source 1..888: mol_type = other DNA
  CDS 1..888: protein_id = 138; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNTRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctctgagccctctgtctcagcgttttgaacgcattgcggtgcagccgctgaccggtgtcctgggtgcagagatcaccggtgtagacctgcgtgaaccactggatgatagcacctggaatgaaatcctggatgcgtttcatacctatcaggttatctacttcccgggtcaggcgatcactaacgaacagcacatcgcgttctctcgccgcttcggcccggtagatccggtcccgcatctgaaatctattgaaggctaccctgaggttcagatgatccgccgtgaagctaacgaaagcctgcgcgtcatcggtgacgactggcacaccgattccaccttcctggacgcgccaccggcggctgtggtaatgcgcgcaattgacgtgccggagcatggtggtgacaccggtttcctgtctatgtacaccgcttgggaaaccctgagcccgactatgcaggcaactatcgaaggtctgaatgtggtacatagcgcgactcgtgtgttcggtagcctgtaccaggcccagaacacccgctggagcaacacgtccctgaaagttatggatgtagacgcaggtgaccgtgaaaccgttcacccgctggtagtgacccacccgggctctggtcgcaagggtctgtatgttaaccaggtgtactgtcagcgtattgaaggcatgaccgatgcggaatccaagccgctgctgcagtttctgtatgaacatgcgacccgtttcgattttacctgccgtgtccgttggaaaaaagatcaggtgctggtttgggacaacctgtgcaccatgcaccgtgctgttccggattacgctggtaaattccgttacctgacccgtactaccgtgggcggcgttcgcccggctcgctaa

SEQ ID NO: 140 - DNA, 888 bp, synthetic construct
  source 1..888: mol_type = other DNA
  CDS 1..888: protein_id = 138; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNTRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgagtcctctctctcagcgttttgagcgcatcgcggtccaacccctgaccggtgtcctgggcgcggagattaccggtgtcgacctgcgcgagcctcttgacgattccacatggaatgagatcctggacgctttccatacttaccaagttatatacttccctggccaggccatcactaacgagcaacacatcgctttctcaaggcgcttcggaccagtcgacccggtgccgcatctgaagtccatagaggggtatccggaggtccaaatgatcaggcgcgaagctaacgagtcgctgcgcgtcatcggtgacgactggcatacggacagcaccttcctggacgcccccccagccgccgttgtgatgagggcaatcgatgtgccagagcacggtggcgataccggtttcctttcgatgtacacagcatgggagacgctgtccccgaccatgcaggcgacaatcgagggattgaacgtggtgcactccgctacaagggttttcgggagtctctaccaggcgcagaatacaaggtggtcaaatacgtcattgaaggtgatggacgtggacgctggcgaccgggagacggttcatcccttggttgtcacgcacccaggctcgggcaggaagggcctgtacgtgaaccaggtgtactgccaacgcatagagggcatgacagacgccgagtctaagcctcttttgcagtttctgtacgagcacgcaactcgtttcgacttcacctgtcgcgttaggtggaagaaagatcaggttctcgtgtgggacaatctctgtaccatgcaccgggctgttcctgactacgctgggaagtttaggtatttgacacgtacgacagtcggcggggtccggcccgccagatga

SEQ ID NO: 141 - DNA, 888 bp, synthetic construct
  source 1..888: mol_type = other DNA
  CDS 1..888: protein_id = 138; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNTRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgcgctctcgccattgagccagcgctttgaaagaattgctgtacagccactaacgggagtcctgggggcggaaatcactggagtagatctaagagagccattggatgactccacttggaatgaaatattggacgcgttccacacttaccaggtaatttattttcccggtcaggcaatcaccaacgaacaacatattgcttttagcaggcgctttggacctgtcgaccctgttcctcacttaaaatcaattgagggttatccagaagttcagatgatacggcgcgaagccaatgaatcactaagagtaattggggacgactggcataccgacagtacctttcttgacgcgccgcctgcagccgtagtgatgagagccatcgatgtgccagaacacggtggggatacgggcttcttatctatgtatacagcatgggaaacgttatcgccgactatgcaggcgacaattgaaggccttaacgtggttcacagtgcaacacgggtgtttgggagtttgtaccaagcacagaatacaagatggagcaatacctctttaaaggtaatggatgttgatgccggtgaccgtgagactgtacatccgctcgttgtaactcaccccggaagtggacgtaagggtttatatgttaatcaggtatattgccaacggatagaaggcatgactgatgctgaaagtaaaccgctgcttcagttcttatacgagcacgcaacgcgttttgatttcacctgcagggtgcgttggaagaaagatcaggtcttggtctgggacaacctatgtaccatgcacagagccgtgcccgactacgctggtaaatttaggtatctcacgcgcactactgtgggtggagtgcgaccagcccgatga

SEQ ID NO: 142 - protein, 295 aa, synthetic construct
MHAALSPLSQRFERIAVQPLTGVLGAEITGVDLREPLDDSTWNEILDAFHTYQVIYFPGQAITNEQHIAFSRRFGPVDPVPHLKSIEGYPEVQMIRREANESGGVIGDDWHSDSTFLDAPPAAVVMRAIDVPEHGGDTGFLSMYTAWETLSPTMQATIEGLNVVHSATRVFGSLYQAQNQRWSNTSLKVMDVDAGDRETVHPLVVTHPGSGRKGLYVNQVYCQRIEGMTDAESKPLLQFLYEHATRFDFTCRVRWKKDQVLVWDNLCTMHRAVPDYAGKFRYLTRTTVGGVRPAR

SEQ ID NO: 143 - DNA, 888 bp, synthetic construct
  source 1..888: mol_type = other DNA
  CDS 1..888: protein_id = 142; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNQRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgagcccgctgtcccaacgctttgaacgtattgctgtgcagccgctgaccggtgttctgggtgctgagatcaccggtgttgatctgcgtgagcctctggacgactctacctggaacgaaatcctggacgcttttcacacttaccaagttatttacttcccaggtcaggcgattaccaacgaacagcacatcgcgttctctcgtcgtttcggcccggtggatcctgttccgcatctgaaatctattgaaggttacccggaggttcaaatgatccgtcgcgaagccaacgaatctggcggtgttattggtgacgattggcacagcgactctactttcctggatgcgccgccagcagcagtagttatgcgtgctattgacgttccggaacatggtggcgacaccggcttcctgagcatgtataccgcctgggaaactctgtctccgactatgcaggcgaccatcgaaggcctgaacgtggttcactctgccacccgtgtgttcggctctctgtaccaggcgcagaaccagcgttggtccaacacttctctgaaagttatggacgttgacgcgggtgatcgtgaaaccgttcacccgctggttgtcacgcatccgggttccggtcgtaaaggtctgtacgtgaaccaagtttactgccagcgtatcgaaggtatgaccgacgcggaaagcaaaccgctgctgcagtttctgtatgagcacgcgacccgtttcgattttacctgccgtgtacgttggaaaaaagaccaggttctggtttgggacaacctgtgtaccatgcaccgtgctgtgccggattacgccggcaaattccgttacctgactcgtaccactgttggtggcgttcgcccggctcgttaa

SEQ ID NO: 144 - DNA, 888 bp, synthetic construct
  source 1..888: mol_type = other DNA
  CDS 1..888: protein_id = 142; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNQRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagccctgtcacctctctcgcaaaggttcgaaagaatcgcggtccaacccctgacaggcgttctcggtgccgaaattacgggcgttgacctgcgcgagccgctggacgatagcacatggaatgagattcttgatgccttccatacttaccaagtcatctacttcccgggccaagctatcaccaacgaacaacacattgcattcagtcggcggttcggcccggtcgaccctgtgccacaccttaagagcattgaggggtaccccgaggtgcaaatgattcgcagggaggccaatgagtctgggggggttataggggacgactggcacagtgactccaccttcctcgacgcgccgcccgcagccgtggtgatgagggctattgacgttccggagcacgggggggatacagggttcctgagcatgtacaccgcgtgggagaccctgagtcccacgatgcaggccactatcgaggggctcaatgtggttcactccgcaaccagggtgttcggatccctctaccaggcacagaaccagcggtggtccaacacgtcgttgaaggttatggacgttgacgccggcgacagggaaactgtccatccgttggtggtcacccatccgggctccggacggaagggcttgtatgtcaaccaggtgtactgccagaggattgagggtatgaccgatgcggagtcgaaaccacttcttcagttcctttacgaacacgccaccaggtttgactttacatgtagggtgcgttggaagaaggaccaagtcctggtctgggacaacctctgcaccatgcaccgtgcagtcccggactatgcaggcaagttcaggtaccttacgagaacgacagtgggaggggttagacctgccaggtga

SEQ ID NO: 145 - DNA, 888 bp, synthetic construct
  source 1..888: mol_type = other DNA
  CDS 1..888: protein_id = 142; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNQRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctctttctcctctttctcaaagattcgaaagaattgctgttcaacctcttactggtgttcttggtgctgaaattactggtgttgatcttagagaacctcttgatgattctacttggaatgaaattcttgatgctttccatacttatcaagttatttatttccctggtcaagctattactaatgaacaacatattgctttctctagaagattcggtcctgttgatcctgttcctcatcttaagtctattgaaggttatcctgaagttcaaatgattagaagagaagctaatgaatctggtggtgttattggtgatgattggcattctgattctactttccttgatgctcctcctgctgctgttgttatgagagctattgatgttcctgaacatggtggtgatactggtttcctttctatgtatactgcttgggaaactctttctcctactatgcaagctactattgaaggtcttaatgttgttcattctgctactagagttttcggttctctttatcaagctcaaaatcaaagatggtctaatacttctcttaaggttatggatgttgatgctggtgatagagaaactgttcatcctcttgttgttactcatcctggttctggtagaaagggtctttatgttaatcaagtttattgtcaaagaattgaaggtatgactgatgctgaatctaagcctcttcttcaattcctttatgaacatgctactagattcgatttcacttgtagagttagatggaagaaggatcaagttcttgtttgggataatctttgtactatgcatagagctgttcctgattatgctggtaagttcagatatcttactagaactactgttggtggtgttagacctgctagatga

SEQ ID NO: 146 - DNA, 888 bp, synthetic construct
  source 1..888: mol_type = other DNA
  CDS 1..888: protein_id = 2; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSVKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgccgcacttagtccacttagccaaagattcgagagaatagccgtgcaaccccttactggagtccttggtgctgagattacaggtgtcgatttgagagaacctttggatgattctacatggaatgagatattggatgccttccatacctatcaggtaatttattttcctggacaggcaattaccaatgagcagcatatagctttttccagaaggttcggccccgttgatcccgtgccacaccttaagtctattgagggttatccagaagttcagatgataagaagagaggccaatgaaagtgggagggttataggcgatgattggcacactgattctacattcctcgatgcaccacccgctgcagtcgttatgagagcaatcgacgttccagagcacgggggagacacaggattcctgagcatgtataccgcatgggaaacattgtctcccacaatgcaagccacaatagagggtttgaacgttgtccattctgcaactagagtgttcggaagtctttaccaagctcaaaatcgcaggttttctaatactagtgttaaagttatggatgtggacgcaggagatagggaaacagtacacccactcgtggttacccatccagggtcaggaaggaagggactttacgttaaccaggtctactgtcagcgtatcgagggaatgactgatgcagagtctaagccattgcttcaatttctctatgagcacgctactagatttgattttacatgtagggttaggtggaagaaagaccaggtcttggtgtgggacaacctttgcacaatgcaccgcgcagtgcctgattatgctggaaagttccgctaccttacccgcacaactgttggaggcgtcaggccagcaagatga

SEQ ID NO: 147 - DNA, 888 bp, synthetic construct
  source 1..888: mol_type = other DNA
  CDS 1..888: protein_id = 6; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcattgtctcccctctctcaaaggttcgagcgcattgctgtccaaccccttacaggggttctcggtgctgaaattacaggagttgacctcagagagccactcgacgacagtacctggaacgaaattctcgatgcctttcatacctatcaggtgatatatttcccaggtcaggcaattaccaacgagcagcatattgctttctctagaaggttcggaccagtggatcctgtcccacatttgaagagcattgaggggtaccctgaagttcagatgatccgccgtgaagccaatgagtcaggcagggtgatcggtgatgactggcataccgactcaacctttctggatgcccctccagctgctgttgtcatgagagctattgatgtgcctgagcatggcggtgatactggttttcttagcatgtatactgcctgggagaccctttcaccaaccatgcaagctacaatcgagggacttaacgttgttcatagtgccactagggtgttcggttccctgtaccaggcacaaaacagaaggttctctaacacatccctgaaagtaatggatgtggatgcaggtgacagagaaactgtgcaccctttggtggtgacacatccaggatctggacgtaagggactttatgtcaatcaagtgtattgccaaagaatagaagggatgacagatgccgagagtaagcctttgctccaattcctgtatgaacatgctacccgttttgactttacatgccgtgtccgttggaagaaggatcaggttttggtgtgggataatttgtgtacaatgcatcgcgcagttccagattatgctggaaaattcaggtatctcaccaggacaacagtaggcggggttagacctgcaaggtga

SEQ ID NO: 148 - DNA, 888 bp, synthetic construct
  source 1..888: mol_type = other DNA
  CDS 1..888: protein_id = 10; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSMKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagctctttccccactttcacaaagattcgaaagaattgcagttcaaccattgacaggagtgcttggcgcagagatcactggtgtggatcttagggaaccactcgatgatagtacctggaacgagattctcgacgccttccatacctaccaggtgatctacttccccggacaggcaatcaccaacgagcaacacatcgcttttagtaggcgcttcggtcctgtcgacccagtcccccacctcaagtcaattgaggggtaccctgaagtgcagatgatcagaagagaagctaatgaatctggacgtgttattggtgacgattggcatacagactctactttcctcgacgcacctcctgccgcagttgttatgagagctattgatgtacctgagcatggcggagatactgggttcctttcaatgtatactgcatgggaaacactttctcctactatgcaggctactatcgaaggattgaatgttgttcatagtgccactagagtctttggaagcctttaccaagcccaaaacaggcgttttagcaacacaagcatgaaggttatggacgttgatgcaggcgatcgcgaaacagttcacccattggttgttacacatcctgggagcggaaggaaaggtctctatgtcaatcaggtttactgccaaaggatcgaagggatgacagatgcagagtccaagccactgctccagttcctgtacgagcacgctacccgctttgattttacctgccgcgttagatggaagaaggatcaggtgcttgtttgggacaatctttgtactatgcaccgcgctgtccccgattatgcaggcaagttcagatacctgactaggactacagttggtggggtccgccccgctagatga

SEQ ID NO: 149 - DNA, 888 bp, synthetic construct
  source 1..888: mol_type = other DNA
  CDS 1..888: protein_id = 14; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRFSNTSIKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgccctttctccactttcacaaaggtttgaacgtattgctgtccagcctttgactggtgtgttgggcgctgagattactggagtcgatcttagggaacctcttgatgacagcacatggaacgaaattttggatgctttccatacatatcaagttatctacttcccaggacaggcaataacaaacgagcagcatattgcattttctagaagattcggaccagtggacccagttcctcatttgaaatcaatagaggggtacccagaggtgcagatgattaggcgcgaggcaaacgaaagtggtagagttataggtgacgattggcacactgactctacctttcttgacgcaccaccagccgctgtcgtcatgagggctattgacgtgccagaacacggaggagatactggattcttgtctatgtacacagcttgggagactcttagccccactatgcaagccactatcgaaggactgaacgtcgtgcacagcgctacaagagtttttggctccctttatcaagcccagaaccgcagattcagtaacactagtattaaggtgatggacgttgatgctggagatagagaaaccgtgcacccattggtggtcacacatccaggaagtggaaggaagggactgtacgtaaaccaggtctattgtcaacgcattgagggaatgactgatgcagagtcaaagcctctccttcagtttctctacgaacacgccaccaggttcgatttcacttgtagggttcgctggaaaaaagaccaggtgcttgtgtgggataacttgtgtacaatgcatagggctgtcccagattatgccggtaagttcaggtacctgactcgcacaactgttggaggagttaggcctgcaagatga

SEQ ID NO: 150 - DNA, 888 bp, synthetic construct
  source 1..888: mol_type = other DNA
  CDS 1..888: protein_id = 18; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GAVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctcttagcccccttagtcaaagatttgaaaggattgcagttcaacctctcacaggagtgcttggtgccgagatcacaggtgtagacctcagggaacccctggatgactctacatggaacgaaatactcgatgccttccatacatatcaagtgatttactttcctggtcaggcaataacaaatgagcaacatatagcttttagtaggcgctttggaccagtggacccagtgccacatctcaagtccattgagggctacccagaagtccagatgataaggagagaggctaacgaatccggagctgttataggggatgactggcatactgatagtacatttctggatgcaccacccgctgcagttgttatgagggccatcgatgtgcctgaacacggcggtgacaccgggttcttgagtatgtatacagcatgggagactctttctccaactatgcaggcaactatcgaagggcttaatgtggttcatagtgcaactagagtgttcggatccttgtatcaagctcaaaatagacgctttagtaacacaagcctcaaggttatggacgtcgatgccggagatagggagaccgtacacccactggtcgtaactcatcccggcagtggaagaaaaggtctctacgttaaccaagtatactgccagcgtatagaaggaatgactgatgccgaatcaaaacctttgttgcaattcctttatgagcacgctacacgtttcgattttacatgtagggtgagatggaaaaaagaccaggtacttgtttgggataacctctgcacaatgcatagagcagttccagactatgctgggaaattcaggtacctcactaggactaccgtcggcggtgtgagacctgctcgctga

SEQ ID NO: 151 - DNA, 888 bp, synthetic construct
  source 1..888: mol_type = other DNA
  CDS 1..888: protein_id = 22; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GDVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctcttagcccactgtcccagaggttcgaaagaatcgctgtgcagcctcttacaggagttctcggcgctgaaattacaggtgtcgaccttcgtgaaccccttgacgattcaacctggaacgagattctcgatgccttccacacctaccaggttatttacttcccaggtcaggcaatcactaacgaacaacatatagctttttccagaagatttggacccgtggatccagtgccacatttgaaaagtatagaaggttatccagaggtccaaatgattaggagggaagccaacgaatcaggcgatgttattggagatgactggcataccgacagcaccttcctcgacgctcctcctgcagctgttgtgatgcgcgcaatagatgttccagagcacggaggcgataccggatttttgtcaatgtacactgcttgggaaactctgagtcctaccatgcaggcaaccattgagggtctcaacgtagtgcatagcgccacccgcgtctttggttcattgtaccaagcacaaaataggaggttctccaacacttccttgaaggtaatggatgttgatgccggtgatcgtgaaaccgttcatcctctcgttgtgactcatccaggaagtggacgtaagggactttacgtcaaccaagtttactgccagagaattgaaggtatgactgatgctgaatcaaaaccactgctccagttcttgtacgaacatgccactagatttgactttacctgtcgtgttagatggaaaaaggatcaagtgttggtttgggataatctctgtaccatgcacagggctgtgccagattatgccggcaagtttaggtatctgaccagaaccactgttggtggggtcagaccagccagatga

SEQ ID NO: 152 - DNA, 888 bp, synthetic construct
  source 1..888: mol_type = other DNA
  CDS 1..888: protein_id = 26; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GLVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgccctgagccctctgtcacagaggtttgaaagaatcgccgtccaaccactgactggcgttttgggagctgagattactggagtggatctgagggaaccactcgatgattcaacttggaacgaaattcttgacgcttttcacacttatcaggtcatctactttccaggtcaggctatcacaaacgagcaacatattgctttcagcaggagatttgggcccgttgatccagttcctcacctcaaatctatcgaagggtacccagaggtccaaatgattcgcagagaagctaacgaatctggattggttattggagatgattggcacactgatagtacattcttggatgccccccctgcagctgttgtgatgagggctatcgatgttcccgaacacggaggagatacagggttcctttctatgtatacagcctgggagacattgtctccaactatgcaggcaactattgaaggacttaacgtggtccactctgccactagggttttcggatctctttatcaggctcagaataggagatttagcaatacaagtctgaaagtcatggacgtagatgctggcgacagagaaacagttcaccccctggtggttactcacccaggatctgggcgtaaaggcctttatgttaaccaagtatactgccagaggattgagggtatgactgacgcagagtccaaacccctccttcagttcctttacgaacatgctacaagattcgactttacatgccgtgttcgctggaaaaaagaccaagttttggtgtgggataacctctgtacaatgcaccgtgcagtgcctgactacgccggtaagttccgctatcttactagaactactgtaggaggggttagacccgctcgttga

SEQ ID NO: 153 - DNA, 888 bp, synthetic construct
  source 1..888: mol_type = other DNA
  CDS 1..888: protein_id = 30; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcattgagtcccctctctcagaggtttgagcgtattgccgttcagcctctgactggagtattgggagctgaaattactggcgttgatctccgtgagcctcttgacgatagtacatggaacgaaatccttgacgcatttcacacataccaagtcatctactttccaggacaggctattactaacgaacaacatattgcattttcaagaagattcggtccagttgatccagttcctcacctgaagagcattgagggttaccccgaggttcagatgatacgcagagaggctaacgagtcaggaagagttatcggagatgattggcacacagatagcacttttcttgatgccccaccagctgctgtcgtaatgagagccattgatgttcctgaacacggcggcgacactggtttcctctccatgtatactgcatgggaaacccttagcccaactatgcaagcaactatcgaaggactcaatgtcgtgcacagtgcaactagagtgtttggctccctctatcaagcacagaaccgcagatggagtaatacctctttgaaggtcatggatgtggacgctggagatagggaaacagtgcatcctctcgttgttactcatccagggagcggtagaaaaggattgtacgtgaaccaagtttactgtcagcgcattgaaggtatgaccgacgcagaatctaagcctctgttgcaatttctctatgagcacgcaaccagatttgatttcacatgtagagtgaggtggaagaaagatcaagttcttgtctgggacaacctttgtacaatgcacagagccgtgccagattacgccggaaaattcagatatctcactagaactactgtcgggggcgtgagacctgcaaggtga

SEQ ID NO: 154 - DNA, 888 bp, synthetic construct
  source 1..888: mol_type = other DNA
  CDS 1..888: protein_id = 34; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRFSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagccctttctcccctttcacagcgctttgagaggatagctgttcaaccactcaccggcgtattgggcgctgagataaccggagtggacctcagggagccacttgatgattctacctggaacgaaatactcgatgccttccatacctaccaagtgatctacttcccagggcaagctatcaccaatgagcagcacattgctttttcccgtcgtttcgggccagtggatccagttcctcatcttaagtctatcgaaggatatcccgaagttcagatgataagaagggaggcaaacgaatcattgagagttatcggagatgactggcataccgactccacttttcttgatgcaccacctgccgcagtggtcatgagggcaatcgatgtacccgaacatggaggagatacaggtttcctctctatgtataccgcctgggaaactttgtctccaacaatgcaagctaccatcgaggggctcaatgtcgttcacagtgcaacacgtgtgtttggtagcctctatcaagcccagaatcgcagattttctaacacttctttgaaggtgatggatgtggacgcaggcgacagggagaccgtgcatccactggttgtcacccatcctgggtcaggtaggaaagggctttatgttaatcaggtgtactgtcaaaggattgaaggtatgacagatgcagagagcaaacctcttctgcaatttctctacgaacatgctacaagattcgatttcacatgccgtgttagatggaaaaaagatcaagtactcgtgtgggacaacctgtgtactatgcacagggctgtacccgactatgccggaaaatttagatatctcacaaggaccacagtcggtggtgtcagacctgccaggtga

SEQ ID NO: 155 - DNA, 888 bp, synthetic construct
  source 1..888: mol_type = other DNA
  CDS 1..888: protein_id = 38; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgccttgagccctctgtcacagagattcgaaagaatcgcagttcaaccacttactggagtcctgggtgctgagattactggtgtcgatcttagagagccactcgatgactccacttggaacgagattttggatgctttccatacttatcaagttatatattttccaggccaggccataacaaacgagcagcatattgccttctctcgtagattcggaccagtggatcccgttcctcacctgaaatcaatcgagggctatccagaggtccaaatgatccgcagagaggcaaacgagtcaggcggtgttatcggcgatgactggcacaccgactccactttcttggatgctcccccagcagcagttgttatgagagctattgacgtgcccgaacacggtggagatacaggatttctctccatgtatacagcctgggagacactttcccccacaatgcaagcaaccattgagggtttgaatgtggtacattctgcaacccgtgtctttggaagtctgtatcaggcacagaacagaaggtggtccaatacatctttgaaggtcatggatgtggatgccggagatagagaaaccgttcatcctctcgtggtgacacatcccgggtcagggcgcaagggtctctacgttaatcaagtttactgccagcgtattgaagggatgacagatgcagaaagtaagcccttgcttcagttcttgtatgaacacgcaactaggttcgactttacctgtcgtgttagatggaagaaagatcaggttcttgtgtgggacaacctttgcactatgcatagagctgtgcccgattacgccggaaagtttagatatcttactaggactaccgtaggcggagtaaggcccgcaagatga

SEQ ID NO: 156 - DNA, 888 bp, synthetic construct
  source 1..888: mol_type = other DNA
  CDS 1..888: protein_id = 42; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcattgagtcccttgtcccaaaggttcgaaagaatcgccgtgcaacctcttactggagtgcttggagctgaaattaccggtgtcgatttgcgtgaaccacttgatgactctacctggaatgagatcttggacgcatttcacacataccaagtgatatactttcctggtcaagccatcactaacgagcagcatattgccttcagcaggaggtttggcccagttgatccagtgccccacttgaaatctatagagggttacccagaagttcagatgatcagaagggaagctaacgagtctctccgcgtcataggagatgattggcatactgactccacatttctcgacgcacctcccgcagccgtcgtaatgcgcgccattgacgtgcctgagcatgggggtgataccggtttcctctcaatgtatacagcatgggagactctttcacctactatgcaggccactattgagggactcaacgttgtgcatagcgctacacgcgtttttggttcactgtatcaggctcaaaatagaaggtggtctaacacctcccttaaggttatggatgtggatgccggtgacagagaaacagtacatcctcttgttgtgacacatccagggtccggtcgcaagggactttacgttaaccaagtgtactgccaaaggattgaaggaatgactgacgcagagtcaaagcccctcctccaatttttgtacgaacatgctacaagatttgattttacttgtagagtgcgctggaaaaaggatcaagttttggtttgggataatttgtgcaccatgcatcgcgcagtccctgattatgccggcaaattcaggtatcttacaagaacaaccgtgggtggagtccgccctgctcgctga

SEQ ID NO: 157 - DNA, 888 bp, synthetic construct
  source 1..888: mol_type = other DNA
  CDS 1..888: protein_id = 46; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgccttgtcccctttgagccaacgctttgaaagaatagctgtgcaacccttgacaggtgtgttgggggccgagatcaccggtgtggacttgcgtgagcccctcgatgattccacttggaatgagattctggacgccttccacacttaccaagttatttatttccctggtcaggccatcacaaacgagcaacacattgcattctctcgccgtttcggaccagttgatcccgtcccccacttgaaatctattgaggggtaccctgaagttcaaatgataagacgtgaggcaaatgagtctggaggggttattggcgatgactggcattccgattctacttttctcgatgcacctcctgccgcagtggtgatgagggccattgatgtgccagaacatggcggggataccggatttctttccatgtataccgcatgggagaccctttcaccaacaatgcaagctacaatagaaggtcttaacgtggttcatagtgcaacaagagttttcgggagtctctaccaagcacaaaatcgtaggtggagcaatacctctttgaaggtgatggacgtcgatgcaggggatagggaaaccgtgcatccacttgtagttacacacccagggtctggtaggaaggggttgtatgttaatcaagtctactgccagagaattgagggaatgactgatgctgagagtaagcccctcctgcagtttctttatgagcatgccactaggtttgactttacatgcagagtgagatggaagaaggaccaagtgctcgtctgggacaacctgtgtaccatgcatagggccgtcccagattatgcaggcaagtttagatatctgactagaacaaccgtaggtggtgttcgccccgctaggtga

SEQ ID NO: 158 - DNA, 888 bp, synthetic construct
  source 1..888: mol_type = other DNA
  CDS 1..888: protein_id = 50; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagccttgtcaccactttctcaacgctttgagagaatcgcagtacaacctcttactggggttcttggagccgaaataacaggagttgacctgagggaaccacttgacgattctacctggaatgagatacttgatgcttttcatacctatcaagtcatttatttcccagggcaagccattactaacgaacagcacatagctttttcaaggaggtttggaccagtggatcccacccctcatttgaagtcaattgagggctaccctgaggttcaaatgatccgtcgtgaggcaaatgagtccggcggtgtaattggtgatgattggcacactgatagcacattcttggatgcaccaccagctgcagttgttatgagggcaatcgatgtcccagaacacgggggagacactggatttctctctatgtacacagcttgggaaaccctgtctccaactatgcaagctaccattgaaggactgaatgttgttcattctgccactcgtgtgttcggtagtttgtatcaggcacaaaatagaaggtggagtaacacaagtttgaaagtgatggacgtcgatgctggtgatagggagacagttcatcccctggtggttactcatcctggttccggacgtaaaggtctgtatgttaaccaggtttactgtcaaaggattgaggggatgactgatgctgagagcaaaccattgcttcaattcttgtatgagcatgcaacaagatttgattttacttgtcgtgtcagatggaagaaggatcaagttttggtgtgggataacctttgcacaatgcacagagcagtccctgattatgctggcaaatttaggtatttgactaggacaaccgtgggaggagttagaccagccagatga

SEQ ID NO: 159 - DNA, 888 bp, synthetic construct
  source 1..888: mol_type = other DNA
  CDS 1..888: protein_id = 54; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcactcagtccactctcccagagattcgagagaattgcagtccagccactgactggagttcttggtgctgagattactggcgttgatctcagggaacccttggatgattcaacttggaatgagatacttgacgctttccatacttaccaggttatatacttccctggccaagccattacaaatgagcaacacatcgctttttcccgcagattcggccctgtcgacccagttccacacttgaaatcaatagaaggttaccccgaggtccaaatgataagaagagaagctaacgagtctggcggtgtcattggtgatgactggcacaccgattcaacatttctggacgcaccccctgctgctgtcgtgatgagagctattgatgttccagagcacggaggtgatacagggttcttgtccatgtatacagcctgggagactttgtccccaacaatgcaggcaactatagaaggattgaatgtggttcacagtgcaaccagggtttttgggagcctgtaccaagcccagaactggcgctggtctaacacaagtctcaaggttatggatgtggatgctggcgatagagaaacagttcaccctctcgtggttactcatcctggttcaggtaggaagggcctgtacgttaatcaagtttactgccaaaggatcgaaggcatgacagacgctgagtcaaaacctctgcttcaatttttgtacgaacacgcaactagatttgactttacctgtcgcgtgaggtggaagaaagaccaagttttggtatgggataatctttgcactatgcatagggctgtgcctgattacgcaggaaagttcaggtacctgactagaaccaccgtcgggggagtgaggccagcaagatga

SEQ ID NO: 160 - DNA, 888 bp, synthetic construct
  source 1..888: mol_type = other DNA
  CDS 1..888: protein_id = 58; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctttgtcccctctgtcacaaaggttcgagagaatcgctgttcagccccttactggtgttcttggtgccgagattacaggtgttgacttgagggagcctcttgatgacagtacctggaatgagattcttgacgcttttcacacctaccaagttatctactttcccgggcaagcaattactaatgaacaacacatagccttctctagaagatttggccctgttgaccctgtgcctcatttgaaaagtatcgaaggatatcccgaggttcaaatgattagaagggaggctaatgaatccggaggtgtgataggagatgattggcataccgatagcactttcctggatgcacctcctgcagctgttgttatgcgtgctattgatgtgccagagcatggaggggatactggttttttgagtatgtacaccgcttgggagacattgtcaccaactatgcaagcaaccattgagggactcaatgttgttcactctgctaccagggtctttggttctctttaccaggcacaaaacatgagatggtccaacaccagtcttaaggtaatggacgtggatgccggagaccgtgaaaccgtccatccacttgtcgttacacatcctggaagcggaaggaaaggtctctatgttaatcaggtttattgccaacgcattgagggaatgacagatgccgaatccaaaccattgctgcaatttctttacgaacatgcaacacgctttgatttcacctgcagggttagatggaagaaggatcaggtacttgtgtgggataatctttgtactatgcacagggccgtccctgattatgccggaaagttcagatatttgacaagaaccaccgtgggaggagtaaggccagccaggtga

SEQ ID NO: 161 - DNA, 888 bp, synthetic construct
  source 1..888: mol_type = other DNA
  CDS 1..888: protein_id = 62; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGCDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctttccccactgagtcaacgcttcgagaggattgctgtgcagcccttgacaggagttctcggagctgagattaccggggttgaccttagggaacctctcgacgattctacctggaatgagatcttggatgctttccatacttaccaggtgatatacttccctggacaggcaattactaacgagcaacacatcgcttttagcagaaggttcggaccagtcgatcccgtgccacatcttaagagcatagagggctatcctgaagtacaaatgataagaagagaggccaatgagtcaggcggtgtcattggctgcgattggcatactgactccaccttcctggacgcacctcctgccgcagttgtcatgagggccatagatgtgcctgagcatggaggagatacaggattcttgtcaatgtatactgcatgggagaccctttcccccaccatgcaggccacaattgagggcttgaacgtggtccattctgctacccgcgtatttggatcattgtaccaggcacagaatagaaggtggtcaaatacaagcttgaaggttatggatgttgatgcaggagacagagagacagtgcaccctctcgttgtgacacatcctggaagtggcagaaaggggttgtacgtgaatcaggtgtactgtcaaagaatagaaggcatgactgacgctgagagcaaacccttgctccagttcctttacgaacatgcaaccaggtttgatttcacatgcagagttaggtggaaaaaagaccaagtattggtgtgggacaatctttgtacaatgcatagggctgtaccagattacgctgggaagttccgctacttgactaggacaaccgtgggaggtgtgcgccccgccaggtga

SEQ ID NO: 162 - DNA, 888 bp, synthetic construct
  source 1..888: mol_type = other DNA
  CDS 1..888: protein_id = 66; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDEWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgccctttctccactttcacaaagatttgagaggattgctgtccaaccacttaccggtgtgctcggtgctgagattacaggagttgatctcagagaaccactggacgactctacatggaacgagatccttgacgctttccacacttaccaggtaatctatttccctggccaggccattacaaatgagcaacatatcgcattctcacgccgtttcggacctgttgatcctgttccccatctcaagtcaatagaaggttatcctgaggtgcaaatgattcgtagagaagcaaacgagtccgggggggtgataggggacgaatggcataccgattccacatttcttgatgccccacctgccgcagttgttatgcgtgcaattgatgtacccgagcatggcggtgacaccggatttttgagtatgtacaccgcatgggaaactctttccccaacaatgcaagccactattgaaggactcaacgtggttcacagcgcaacaagggttttcggttctctgtaccaggctcaaaacagacgttggtctaatacaagccttaaagtcatggacgttgatgcaggagatagggaaaccgtacatccactcgtggtgacacatccaggcagtggaaggaagggactttacgttaatcaagtttactgtcaacgtattgaaggaatgacagatgccgaatctaaacctttgctgcaattcctgtatgaacacgctaccagatttgatttcacttgcagggttagatggaaaaaggatcaggtcctggtgtgggacaacttgtgcactatgcatcgtgctgtcccagattacgccggaaagttccgctacttgactcgcactactgtcggtggagtcaggcccgctcgctga

SEQ ID NO: 163 - DNA, 888 bp, synthetic construct
  source 1..888: mol_type = other DNA
  CDS 1..888: protein_id = 70; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN CVYCQRIEGMTDAESKPLLQFLYEHATRFDFTCRVRWKKDQVLVWDNLCTMHRAVPDY AGKFRYLTRTTVGGVRPAR
atgcatgctgcattgtctccattgtcccagcgctttgagagaatcgccgttcagcctcttacaggtgttctgggagcagagattaccggggttgacttgagagagcctttggacgattcaacatggaatgagatcctggacgctttccacacttaccaagtaatctatttccctggacaagccatcacaaacgaacagcacatagcattttccaggagatttgggcctgtagatccagttccccatcttaagtcaattgagggctatcctgaagtacagatgatcagacgtgaagcaaatgagtcagggggcgtgataggtgacgattggcacactgactcaacatttctcgatgcaccccctgccgcagttgttatgagagctattgatgtccctgaacatggtggcgatacaggctttcttagtatgtataccgcctgggagaccctttcacctaccatgcaagctacaatcgaaggattgaatgttgtccacagtgctactagagtttttggttcattgtatcaggcacagaacagaaggtggtccaacacaagcttgaaggtcatggatgtagacgcaggagatagagagacagtgcacccactcgtggtgacacatcccggttccggtaggaaaggactctacgtaaactgcgtttactgccagcgtatagaaggtatgaccgatgccgaatcaaaacctttgttgcagtttctgtatgagcacgctactagatttgattttacctgcagggtcaggtggaaaaaagatcaggtgctcgtgtgggacaatctgtgcactatgcatcgcgcagtgcccgactatgctgggaagtttagatacttgactagaaccacagtcggtggcgtgcgccctgccagatga

SEQ ID NO: 164 - DNA, 888 bp, synthetic construct
  source 1..888: mol_type = other DNA
  CDS 1..888: protein_id = 74; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN PVYCQRIEGMTDAESKPLLQFLYEHATRFDFTCRVRWKKDQVLVWDNLCTMHRAVPDY AGKFRYLTRTTVGGVRPAR
atgcatgctgcccttagtccactgagccaaaggtttgagcgtattgctgtccaaccacttaccggcgtccttggcgcagaaattacaggggtggaccttagagagccattggatgatagtacctggaatgaaattcttgatgctttccacacttatcaagtcatctattttccagggcaggccattacaaacgagcaacatatcgccttctctcgtaggtttggaccagttgatcctgtaccacatctcaagtctattgagggatatcctgaggtccaaatgataagaagggaggccaatgagagtggtggcgttatcggcgatgactggcatactgactctacattcttggatgcccctcccgcagccgttgtaatgcgtgctattgatgtgccagaacacgggggagatactggctttctcagtatgtataccgcatgggaaaccctctcacctaccatgcaagctacaattgagggcctgaacgtagtacactcagccacaagggtgttcggaagtctgtatcaggcccaaaacagacgttggtccaacaccagcttgaaagttatggacgttgatgcaggagacagagagacagttcatccattggtcgtcacacatccaggcagtggccgtaaggggctctacgtgaacccagtgtactgccagcgtatagaagggatgactgacgccgagtcaaaacccttgcttcaattcttgtatgagcacgctacaagattcgatttcacttgcagagttcgctggaaaaaggaccaggtcctcgtttgggacaatttgtgtacaatgcatagagcagttccagattatgcaggcaagtttaggtacctcactcgcaccaccgttggaggtgtgagacctgcacgttga

SEQ ID NO: 165 - DNA, 888 bp, synthetic construct
  source 1..888: mol_type = other DNA
  CDS 1..888: protein_id = 78; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNDRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcattgagtcctctcagccaaaggtttgagcgtatagccgtgcaaccactcactggagtcctgggcgcagaaattactggagtggatttgagggaacctttggatgattctacatggaacgagatcctcgacgcatttcatacctaccaggttatttattttccaggccaggccattaccaacgaacaacacatagcattttctaggagatttggtccagtggacccagtaccacacctgaagtccatagagggataccctgaggtgcagatgattcgcagggaggctaacgaatccctccgtgttataggggatgattggcacacagattctacctttttggatgcaccacctgctgctgttgttatgagagcaatagatgtgcccgagcatggaggagatactggcttcttgtctatgtatacagcatgggagaccctttccccaactatgcaagcaactattgaaggacttaacgttgttcactctgctactagggtttttggatctttgtatcaggcacaaaatgacagatggtctaacactagtcttaaagtgatggatgtggatgctggtgacagagaaactgttcatccactcgtcgtgacccacccagggtctggaagaaaaggactctatgtcaaccaagtgtactgtcaaagaatagaagggatgaccgacgcagagagcaagccacttcttcaatttttgtatgagcacgccacccgctttgattttacctgtagggtgagatggaaaaaagaccaggtccttgtatgggataatctgtgcactatgcacagggctgttcctgattatgcaggtaaatttaggtacttgacaagaacaaccgtcggaggagttaggcccgctagatga

SEQ ID NO: 166 - DNA, 888 bp, synthetic construct
  source 1..888: mol_type = other DNA
  CDS 1..888: protein_id = 82; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNERWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagcactgagtcctcttagtcaaaggtttgagagaattgctgttcagcctctgacaggggtccttggtgccgagataacaggtgttgatcttagggagcctctcgatgatagcacatggaacgagattttggatgccttccacacctatcaagttatttactttcctggtcaagccatcactaacgaacaacatattgccttcagcaggagatttggtccagttgacccagtcccacatctgaagagtattgaggggtatcccgaggttcagatgattaggagggaagctaacgaatcattgagggtcattggagatgactggcatacagatagtactttcctggacgctccaccagcagctgtagttatgagagccatagatgttcccgaacatggaggtgatactgggttcttgtccatgtacaccgcctgggaaacactgagccccactatgcaagctactattgaaggacttaatgtagtccattcagccacccgtgtctttggtagcctctaccaagcacaaaatgagagatggagtaatacaagccttaaagttatggatgttgatgccggggatcgtgagaccgtgcaccctttggtcgtgacccatccaggtagcggaaggaagggcttgtacgttaatcaagtgtactgtcagaggattgagggtatgacagatgctgaatccaagccacttctgcaattcttgtacgaacatgcaactcgcttcgattttacttgtagagtcaggtggaaaaaagatcaagttttggtctgggacaacttgtgcacaatgcaccgcgcagtccctgattatgcaggcaagtttaggtatttgacaaggactaccgtaggaggggtgaggcctgctagatga

SEQ ID NO: 167 - DNA, 888 bp, synthetic construct
  source 1..888: mol_type = other DNA
  CDS 1..888: protein_id = 86; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNSRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgcactgtctccacttagccaaaggttcgagagaatagctgtccaaccattgaccggcgtccttggggctgaaataactggcgtcgatctcagagagccattggatgatagcacttggaacgaaattttggatgccttccacacataccaagtcatctattttccaggtcaagcaatcacaaacgagcagcacatagccttttctcgtcgttttggtccagttgatccagtgccacatttgaaatcaatagaaggttaccctgaggtccagatgattagacgtgaggccaatgaaagtcttcgtgtgataggtgatgattggcataccgactcaacctttctggacgctccacccgcagctgttgtcatgcgcgctattgatgtgccagagcacggcggagacaccggatttttgtccatgtacaccgcctgggagactttgtccccaacaatgcaggcaaccatagaggggttgaacgtcgtgcactcagctactcgcgtctttggaagcctctaccaagctcaaaattctcgttggagtaacacctcattgaaagtgatggatgttgatgcaggtgatagagaaacagttcatcctctcgtggtgactcacccaggttctggtcgcaaggggctgtatgttaatcaggtttattgccagagaatcgagggtatgacagatgcagaaagcaagcctttgttgcagtttctttatgaacatgccacaagattcgactttacatgtagggttcgttggaaaaaggatcaagtattggtctgggataatctttgtacaatgcacagagccgtccccgactacgctggcaagtttcgttacttgacaagaacaacagttggtggagttaggccagctcgctga

SEQ ID NO: 168 - DNA, 888 bp, synthetic construct
  source 1..888: mol_type = other DNA
  CDS 1..888: protein_id = 90; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNLRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagcactttcccctctttctcaaagattcgaaagaattgcagtgcaacccctcaccggggttcttggtgctgaaatcacaggagtagatttgagagaacccctcgatgattccacttggaacgaaattttggatgcttttcacacttatcaagtgatctattttcctggtcaggccatcactaacgaacagcacattgctttttctagaaggtttggaccagttgatcctgttccacacttgaaaagcatcgaagggtaccctgaagttcagatgataagaagagaggcaaatgaaagcctcagggtcatcggggatgactggcacactgattcaacattccttgacgcccccccagcagccgtagtcatgagagctattgatgtgcccgaacatggtggagacactggctttctttccatgtataccgcatgggagactctctctccaacaatgcaagccacaatagagggccttaatgtcgttcactctgccacacgcgtgttcggttccttgtaccaagctcaaaacctgaggtggagtaacacttcccttaaggttatggatgttgatgccggtgacagggaaacagtgcacccactggttgtgacccatccagggtccggcaggaagggactctatgtgaaccaagtctattgtcaacgcatagaaggcatgactgatgcagaaagtaagccacttcttcagtttctttacgagcacgccactcgcttcgattttacctgccgcgttaggtggaagaaagatcaagttctggtctgggataacctttgcacaatgcacagggctgtgcccgactatgcaggcaaatttagatatctcactcgcaccactgtcggtggagtgagacctgctaggtga

SEQ ID NO: 169 - DNA, 888 bp, synthetic construct
  source 1..888: mol_type = other DNA
  CDS 1..888: protein_id = 94; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagccctttcccctctgagtcaaaggttcgagcgtattgctgtgcaacccctcactggcgttttgggcgcagaaattactggtgttgaccttcgcgaaccattggacgacagcacttggaacgagatccttgatgctttccatacctatcaagttatctactttcctggccaagctatcactaacgagcaacatattgctttcagtcgtagattcggtccagtggatccagtaccccatttgaaaagcattgagggatacccagaagtacagatgattaggagggaggcaaacgagagccttcgcgtcataggcgatgattggcataccgatagcacttttctggacgccccacctgctgctgttgtaatgagggctatcgatgtacctgagcacggaggtgataccgggtttcttagtatgtacaccgcctgggagacactcagtccaacaatgcaagctactatcgagggtctgaatgtcgttcacagtgccaccagagttttcggatccctttatcaagctcagaactggagatggtccaatacatcccttaaagtcatggatgtggatgctggtgatagagaaacagtccatcctctcgtcgtgactcatccaggaagtggaagaaaggggctttatgtgaatcaggtctattgtcaacgcatcgagggcatgactgacgctgaaagtaagccacttcttcaattcctttacgagcacgccactagatttgattttacatgtagagttagatggaaaaaagaccaggtgcttgtttgggacaacctctgtacaatgcatcgtgcagtgcccgactatgctggtaagttcaggtacctcaccagaacaaccgttggaggtgttagacccgctagatga

SEQ ID NO: 170 - DNA, 888 bp, synthetic construct
  source 1..888: mol_type = other DNA
  CDS 1..888: protein_id = 98; transl_table = 1; translation = MHAALSPLSQRFERIAVQPLTGVLGAEITGVDLREPLDDSTWNE ILDAFHTYQVIYFPGQAITNEQHIAFSRRFGPVDPTPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctcagtccattgtctcaaaggttcgagagaatcgctgttcaaccccttactggtgttcttggagccgaaataactggtgtagacctgcgcgagccactggatgatagcacatggaacgagattcttgacgctttccatacctaccaggtgatttactttcctggtcaggctattacaaacgagcagcatatcgctttcagtcgtcgcttcggccctgttgacccaactccccatttgaaatccattgagggttacccagaagttcaaatgattagacgtgaggcaaacgaatcattgagagtcattggagatgattggcatacagattcaacttttctggatgcaccacctgcagctgttgtgatgcgcgcaattgacgttccagagcacggtggtgatacagggttcctttctatgtatactgcttgggaaacactttctcccaccatgcaagctaccatagagggactcaatgttgtacattccgcaaccagggttttcggcagcctttaccaagctcagaaccgcagatggtcaaacacctctctgaaagttatggacgtagacgctggtgaccgcgagactgtgcatcctcttgttgtcactcatcctggaagcggcagaaaaggtctttatgttaatcaggtttattgccagcgtatcgaaggaatgactgacgcagagtctaagcccttgctgcaatttttgtacgaacatgcaactaggtttgacttcacctgtcgcgttaggtggaagaaagatcaggtccttgtttgggataacttgtgcactatgcatagggcagtgccagattacgcagggaagtttaggtatcttaccaggaccaccgttggtggggtgaggccagcacgctga

SEQ ID NO: 171 - DNA, 888 bp, synthetic construct
  source 1..888: mol_type = other DNA
  CDS 1..888: protein_id = 102; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHADSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgctctttcccctttgtctcagaggttcgaacgtattgctgtgcaaccacttacaggtgttcttggagcagaaattactggagtcgatttgagggagcccctggatgactccacatggaatgagattctcgacgccttccatacttatcaggttatctactttcccggacaggctataacaaacgagcaacacattgctttttctcgccgttttggccctgtggaccctgttccacatttgaagagtatagagggttatccagaggtgcaaatgattagaagagaagctaatgagtccttgagagttataggtgatgattggcatgcagatagcacttttcttgatgcaccaccagcagcagtggttatgagggccattgatgtcccagagcatggaggtgatacaggttttctgtcaatgtatacagcctgggaaactttgagcccaacaatgcaagccaccattgagggtttgaatgtcgttcattccgccacaagggtgtttggctctctttatcaagcacaaaacagaagatggtcaaatacaagcctcaaagtcatggatgtggatgccggtgacagggaaactgtgcatccccttgtggtgactcatccaggatcaggtaggaaagggttgtatgtaaatcaggtatattgccaaagaatagaagggatgactgatgctgaatccaaacctctgctgcaatttttgtatgaacacgctaccaggttcgatttcacatgtagagttcgctggaagaaagatcaagtcttggtttgggacaacctgtgcaccatgcaccgcgccgtgcccgattatgctggtaagtttagatatttgacccgcaccaccgtgggcggagtacgcccagcaagatga

SEQ ID NO: 172 - DNA, 888 bp, synthetic construct
  source 1..888: mol_type = other DNA
  CDS 1..888: protein_id = 106; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHSDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagccctttcacctctctctcaaaggtttgaacgtatcgctgtccagcccctgactggcgtgctgggtgcagagataaccggagtagacttgagggaaccacttgatgactcaacttggaatgagattctcgacgctttccatacataccaggttatttactttccaggacaggcaattactaacgaacagcatatagctttctccagaagatttggaccagttgacccagttccacatcttaagtccatagagggatatccagaggttcagatgataaggagagaagcaaacgagagcttgagagtcatcggtgacgattggcactctgatagcactttcctcgatgcacctccagccgctgttgtgatgcgcgctattgatgttcctgagcacggcggtgatacaggattcctctccatgtatacagcatgggagactttgtcaccaacaatgcaggccaccatcgaaggacttaatgtcgttcacagcgcaacacgtgttttcggcagcttgtaccaagcacagaatcgcagatggagtaacacatctctgaaggtgatggatgtggacgccggcgatcgtgaaacagtccacccactcgttgttacccatcctggatccggccgcaaaggtctttacgtgaaccaggtttactgtcagaggatcgaaggtatgaccgacgccgagtcaaaacctctccttcagtttctctatgagcatgccaccaggtttgattttacctgtagggttagatggaaaaaggatcaagttcttgtgtgggacaatttgtgtacaatgcatagggcagttcccgattatgcagggaaattcagatatttgactagaactaccgttggcggtgttcgtccagccagatga

SEQ ID NO: 173 - DNA, 888 bp, synthetic construct
  source 1..888: mol_type = other DNA
  CDS 1..888: protein_id = 110; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHMDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgcagctttgtcaccactctcacagcgtttcgaaaggattgcagtgcaaccccttaccggtgtgttgggggctgaaattacaggggtggaccttcgtgaacctttggatgattcaacttggaacgaaatccttgatgcatttcatacctaccaagttatttactttcctggtcaagctatcaccaatgagcagcatatcgctttcagcaggaggttcgggcccgttgatcccgtgccacacttgaagtctattgaaggatacccagaagtgcaaatgataagaagggaggctaacgagtcattgcgcgttatcggggacgactggcatatggattctacctttttggacgctccacctgccgctgtcgtgatgagagcaatcgacgtacctgaacatggtggagatactggattcttgtccatgtacacagcttgggagaccttgagcccaactatgcaggcaactatcgagggactcaatgttgtgcacagcgctacacgcgtgtttggttctctttaccaggcacaaaatagacgttggagtaacacatctcttaaggtgatggatgttgatgctggcgatagagaaaccgtgcatcctctggtggttacacatccaggctccggacgcaagggactctacgtcaatcaagtctattgtcaacgtatcgaaggaatgacagatgctgaatctaaacccttgctgcagtttttgtatgagcacgcaactagatttgactttacctgtagagttaggtggaagaaagaccaggtgcttgtgtgggataatctgtgtacaatgcatagggcagttccagattatgctggaaagtttaggtaccttactagaactactgtcggtggagttagaccagccagatga

SEQ ID NO: 174 - DNA, 888 bp, synthetic construct
  source 1..888: mol_type = other DNA
  CDS 1..888: protein_id = 114; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRYDFTCRVRWKKDQVLVWDNLCTMHRAVPDY AGKFRYLTRTTVGGVRPAR
atgcacgctgcactttcacctttgtcacagaggttcgaacgcatcgccgttcaacccctcacaggggtccttggagctgaaatcactggggtggatttgcgcgagccacttgatgatagcacatggaatgagatcttggacgcttttcatacctatcaggtaatctatttccctggacaagctatcaccaatgaacagcacattgcattttctaggagattcggaccagtggaccccgtaccacacctgaaatccattgagggctaccccgaggtgcagatgataagaagggaggcaaatgaaagccttagggttatcggggatgattggcataccgactcaactttcctcgacgccccacctgctgctgttgtcatgagggcaatagatgtgcctgaacatggcggcgacaccggcttcctgtccatgtacaccgcctgggaaaccctcagcccaaccatgcaggctactatagagggacttaacgtggtacacagtgccaccagagttttcggatctctctaccaggctcagaatagaaggtggtcaaacactagcctcaaagtcatggatgtcgatgcaggagatcgtgaaactgtgcatcctctggttgtcactcacccagggtcaggccgcaaggggttgtatgttaatcaagtctactgccagaggattgagggaatgacagacgctgagtccaagcctcttctgcaattcctttatgaacatgcaactagatacgatttcacatgtagagtgcgctggaaaaaagaccaagtacttgtgtgggataacttgtgcacaatgcatagggccgtgcctgattacgcagggaagttcaggtatttgacaaggaccactgtgggaggcgttcgtcccgcacgttga

SEQ ID NO: 175 - DNA, 888 bp, synthetic construct
  source 1..888: mol_type = other DNA
  CDS 1..888: protein_id = 118; transl_table = 1; translation = MHAALSPLSQRFERIAVQPLTGVLGAEITGVDLREPLDDSTWNE ILDAFHTYQVIYFPGQAITNEQHIAFSRRFGPIDPVPHLKSIEGYPEVQMIRREANES GGVIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcacttagcccactctcccaacgttttgagcgcattgctgtgcagccacttactggggtattgggtgctgagataaccggagtcgatcttcgtgagcctttggatgattcaacctggaacgaaatccttgatgcttttcacacttatcaagtgatctattttccaggacaagctataactaatgagcagcacattgcattcagcagaagattcggaccaattgacccagtccctcatcttaagtctattgaaggataccccgaagtccaaatgatcaggcgtgaggccaatgaaagccttagggtgattggagacgattggcacacagactccaccttcctcgatgcaccacccgcagcagttgttatgagagctatcgatgttccagagcatgggggcgatactggattcctttcaatgtatactgcttgggagacactcagtcctacaatgcaagccactatagaaggacttaacgtagtgcattccgccacaagagtttttggttctctttaccaagcacagaacagaaggtggagtaatactagcctcaaggtcatggatgtcgacgctggagaccgcgaaaccgtccaccctcttgtggtcacccatcctgggtctggcagaaaggggttgtatgttaaccaggtttactgtcaacgtatcgaaggaatgacagacgctgaaagcaaaccacttcttcagttcctttatgaacacgcaaccagatttgactttacctgtagagtacgttggaagaaagaccaggtgttggtttgggacaacctgtgtaccatgcatagggctgtgcccgattatgcaggaaagttcaggtacctgaccaggaccactgtgggtggtgttaggcctgctcgttga

SEQ ID NO: 176 - DNA, 888 bp, synthetic construct
  source 1..888: mol_type = other DNA
  CDS 1..888: protein_id = 122; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LGYIGDDWHTDSTFLDAPPAAVVMRAIDVPEHGGDTGFLSMYTAWETLSPTMQATIEG LNVVHSATRVFGSLYQAQNRRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgcagctttgagtcccttgtcccaaaggttcgagcgtatcgcagttcagcctcttacaggagttcttggcgccgaaatcacaggagtggatctcagggaacccttggacgactctacatggaatgagatcttggatgcttttcatacataccaagtgatatactttcctggacaggccattactaatgagcagcacattgctttttcaagacgttttggacccgttgatcctgtgcctcatctcaagagcatagagggctatcctgaagttcagatgataaggcgtgaggctaacgaaagcttgggttatattggcgacgattggcacaccgatagtacttttctggacgccccccctgccgctgtcgtcatgagggctatagacgtgcctgaacatggcggggataccggatttttgtctatgtacactgcctgggagacattgagtcctactatgcaggccaccatagagggacttaatgtcgtgcactcagcaaccagggtgttcggaagcttgtaccaagctcagaatagaaggtggtccaacacttcattgaaggttatggacgttgacgcaggagacagagaaactgtgcatcctttggtggttactcacccaggaagtggccgcaaagggctgtatgttaatcaagtctactgtcagcgtattgagggaatgaccgatgccgaaagcaagcctttgcttcagttcctttatgaacatgcaacacgcttcgattttacttgcagagttagatggaaaaaggatcaagttcttgtttgggataatttgtgcactatgcatcgtgctgttcccgactatgctggaaaattcagatatcttacacgcactacagttggtggagtacgtcctgcacgttga

SEQ ID NO: 177 - DNA, 888 bp, synthetic construct
  source 1..888: mol_type = other DNA
  CDS 1..888: protein_id = 126; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNKRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctttgtcccctctttcacaaagattcgaacgcatcgccgttcagcccctcaccggggtactcggagcagaaattacaggagttgatcttcgcgagcccctcgatgattcaacttggaatgaaatcttggacgcattccatacatatcaggtaatctactttcctggacaagctattactaacgaacagcatatcgctttttcaagaagattcggcccagtggatccagtcccacatcttaaaagcatagagggatatccagaggtccagatgatcagaagggaggcaaacgagtccggcggtgtgatcggagatgactggcattctgattctaccttcttggatgcccctccagccgccgtagtcatgagagcaatcgatgtccctgaacatggaggtgatactgggtttctctctatgtacaccgcatgggaaacactctcacccactatgcaagctacaattgaaggtcttaacgtggttcatagtgctaccagggtgttcggcagtttgtaccaggctcagaacaagaggtggtctaacacctctttgaaggtgatggatgtagacgccggggacagagaaaccgttcatcctttggttgttactcatccaggaagtggcagaaaagggctctacgtcaatcaagtttattgtcagcgcattgagggtatgactgacgccgaatcaaaacctcttttgcaattcctgtatgaacatgccacaaggttcgactttacatgtagagttcgctggaagaaggatcaagtgcttgtgtgggataatctttgtacaatgcatagggctgtaccagattatgccggcaagttccgctacttgaccagaacaactgtaggtggggtgcgccctgctaggtga

SEQ ID NO: 178 - DNA, 888 bp, synthetic construct
  source 1..888: mol_type = other DNA
  CDS 1..888: protein_id = 130; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNMRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgccgcccttagtcctctcagccagaggtttgagcgtatagcagtgcaacctttgaccggggttcttggagccgagatcactggcgttgacttgcgcgagccacttgatgactccacttggaatgagatcttggacgcttttcacacatatcaggtgatttattttccaggacaagccataaccaacgaacaacacattgcattcagtagaaggttcggaccagttgacccagttcctcacttgaagtcaatagaaggttatcctgaagtgcaaatgattagaagagaggctaacgagagtgggggtgtgatcggcgatgattggcatagcgattcaactttccttgatgctcccccagcagccgtggtcatgagagcaatcgacgtgccagaacatggaggcgatacaggttttctttccatgtatactgcatgggaaaccctctctcctaccatgcaggccacaattgaaggactgaatgttgtgcatagcgctactagggtgtttggctctctctatcaggcacaaaacatgcgctggagcaatacatcacttaaggtaatggatgtagatgctggcgatagagaaaccgtgcatccattggtggttactcatccaggctcaggacgtaagggtttgtacgtcaatcaagtttactgccagagaatagaggggatgactgacgctgaatctaagcctcttctccaatttttgtacgagcacgctacaagattcgacttcacatgccgtgttcgctggaaaaaggaccaggttcttgtctgggataacctttgcactatgcatagggctgttccagattacgctggaaaatttaggtacttgactaggactacagtcggtggcgttagacctgctcgctga

SEQ ID NO: 179 - DNA, 888 bp, synthetic construct
  source 1..888: mol_type = other DNA
  CDS 1..888: protein_id = 134; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNWRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgccctctcaccactgagccaaaggttcgagcgtattgcagtccaaccactgaccggtgttctcggagctgagattactggtgtggatctcagggagccactcgacgactccacttggaatgaaattcttgatgcttttcatacctaccaagtcatctactttcctggtcaagctattactaacgaacagcatattgcattctcaaggagattcggaccagtggaccctgttcctcatctcaagagcatcgaaggatatccagaagtccagatgattcgtcgtgaggcaaacgagtcaggtggagtcattggggatgattggcattccgacagtactttcttggatgcaccaccagcagctgttgttatgagggctattgatgttcctgaacatggaggggatacagggttcctctccatgtacacagcttgggaaactttgtctcccaccatgcaggcaacaattgaggggcttaatgtggttcatagcgctacacgtgtttttgggagtctctatcaagcccaaaattggaggtggtcaaacacatcattgaaggtgatggatgtggatgctggggatagagagacagtgcatcctctcgtcgtcactcacccaggaagcggaagaaaggggctttatgttaatcaggtttattgccagagaatcgaaggaatgaccgatgcagaatctaaacccctgcttcagtttctgtatgaacatgcaactaggtttgacttcacttgtagggttagatggaagaaggatcaggttttggtttgggacaacctttgtaccatgcaccgtgctgtccctgactacgctggcaagtttagatatcttacaagaactactgtcggaggtgttcgccctgcccgctga

SEQ ID NO: 180 - DNA, 888 bp, synthetic construct
  source 1..888: mol_type = other DNA
  CDS 1..888: protein_id = 138; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES LRVIGDDWHTDSTFLDAPPAAVVMRAIDVPEHGGDTGFLSMYTAWETLSPTMQATIEG LNVVHSATRVFGSLYQAQNTRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcacgctgctctctcccctctttcccaaagatttgagcgtattgccgtacaaccattgaccggagtcctcggggcagagattaccggggtcgatttgagggaacctctggacgacagcacttggaatgaaatattggatgcttttcacacctatcaagtcatatacttccctgggcaggctatcacaaatgaacaacatattgcatttagcaggagattcggtcctgttgatccagttcctcacctcaagtctatagaagggtaccccgaagttcaaatgattcgccgcgaggccaacgaaagtcttagagtgatcggtgatgattggcataccgatagcactttcttggacgccccacccgctgctgtagttatgcgcgcaatcgacgttccagaacatggtggcgatactggattcctttctatgtataccgcatgggaaaccctctccccaaccatgcaggcaaccatagaaggcctcaacgtcgtgcactcagcaacaagggtttttgggagcctttatcaagcacagaataccaggtggtcaaacacaagccttaaggttatggatgtagatgcaggtgatcgcgagacagtacatcccctcgttgttactcaccccggttcagggaggaagggactttacgttaatcaagtctactgccaaagaatcgaagggatgactgatgcagaatctaagcccttgcttcaatttttgtatgagcacgccaccagattcgattttacttgcagggtgaggtggaaaaaagaccaggtactggtgtgggacaatctgtgtaccatgcatcgtgcagtccctgattatgccggtaagtttagatatctcacacgcacaacagtgggtggtgttcgtcccgcaagatga

SEQ ID NO: 181 - DNA, 888 bp, synthetic construct
  source 1..888: mol_type = other DNA
  CDS 1..888: protein_id = 142; transl_table = 1; translation = MHAALSPLSQRFERIAVQPLTGVLGAEITGVDLREPLDDSTWNE ILDAFHTYQVIYFPGQAITNEQHIAFSRRFGPVDPVPHLKSIEGYPEVQMIRREANES GGVIGDDWHSDSTFLDAPPAAVVMRAIDVPEHGGDTGFLSMYTAWETLSPTMQATIEG LNVVHSATRVFGSLYQAQNQRWSNTSLKVMDVDAGDRETVHPLVVTHPGSGRKGLYVN QVYCQRIEGMTDAESKPLLQFLYEHATRFDFTCRVRWKKDQVLVWDNLCTMHRAVPDY AGKFRYLTRTTVGGVRPAR
atgcatgctgctctcagcccacttagtcagagattcgagagaatagcagtgcaacccttgactggagtcctgggtgcagagattaccggagtcgatttgagagaacctctcgatgattccacctggaatgaaatcttggacgctttccacacttaccaagttatttatttccctggacaagctatcaccaacgaacaacacattgctttttctcgtaggttcggaccagttgacccagtcccacacttgaagagtatcgaaggataccccgaagtccaaatgatcagaagggaggctaatgaatcaggtggggtgatcggtgacgactggcacagcgattctacttttctggatgcaccaccagcagcagttgtcatgcgcgctatcgacgtgccagagcacgggggggatactggtttcctctccatgtacactgcctgggaaaccctgtcaccaacaatgcaggccactattgaaggcctcaatgttgtgcatagcgctacaagggttttcggctctttgtatcaagcccaaaaccagaggtggtcaaatacttcactgaaggttatggacgttgatgctggtgacagagagaccgtccatccacttgtagtcacccatcccggttccggcaggaaaggattgtatgttaatcaggtatactgtcaaaggatcgagggtatgaccgatgcagaatcaaaacctcttttgcagttcctgtatgaacatgcaacaagatttgattttacttgtagggtgcgttggaagaaggaccaggtgctcgtttgggataacttgtgtacaatgcaccgcgcagtgccagactatgctggtaagtttcgttacctcaccagaaccactgtaggcggcgttagacccgcacgttga